SEQUENCE LISTING (38 sequences)

SEQ ID NO: 1 - protein, 206 aa, artificial sequence
  constructed sequence
MKKWTNRLMTIAGVVLILVAAYLFSKPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 2 - DNA, 618 bp, artificial sequence
  constructed sequence
atgaaaaaatggacaaatcgattaatgacaatcgctggtgtggtacttatcctagtggcagcatatttgtttgctaaaccacatatcgataattatcttcacgataaagataaagatgaaaagattgaacaatatgataaaaatgtaaaagaacaggcgagtaaagataaaaagcagcaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaa

SEQ ID NO: 3 - protein, 181 aa, artificial sequence
  constructed sequence
KPHIDNYLHDKDKDEKIEQYDKNVKEQASKDKKQQAKPQIPKDKSKVAGYIEIPDADIKEPVYPGPATREQLNRGVSFAEENESLDDQNISIAGHTFIGRPNYQFTNLKAAKKGSMVYFKVGNETRKYKMTSIRNVKPTAVGVLDEQKGKDKQLTLITCDDLNRETGVWETRKILVATEVK

SEQ ID NO: 4 - DNA, 543 bp, artificial sequence
  constructed sequence
aaaccacatatcgataattatcttcacgataaagataaagatgaaaagattgaacaatatgataaaaatgtaaaagaacaggcgagtaaagataaaaagcagcaagctaaacctcaaattccgaaagataaatcgaaagtggcaggctatattgaaattccagatgctgatattaaagaaccagtatatccaggaccagcaacacgtgaacaattaaatagaggtgtaagctttgcagaagaaaatgaatcactagatgatcaaaatatttcaattgcaggacacactttcattggccgtccgaactatcaatttacaaatcttaaagcagccaaaaaaggtagtatggtgtactttaaagttggtaatgaaacacgtaagtataaaatgacaagtataagaaatgttaagcctacagctgtaggagttctagatgaacaaaaaggtaaagataaacaattaacattaattacttgtgatgatcttaatcgggagacaggcgtttgggaaacacgtaaaatcttggtagctacagaagtcaaa

SEQ ID NO: 5 - protein, 7 aa, artificial sequence
  constructed sequence
LFINEGK

SEQ ID NO: 6 - protein, 10 aa, artificial sequence
  constructed sequence
NTWNLGNNAK

SEQ ID NO: 7 - protein, 16 aa, artificial sequence
  constructed sequence
DGQVIISGSGVTIESK

SEQ ID NO: 8 - protein, 16 aa, artificial sequence
  constructed sequence
EGLTLPVPFNILPSPK

SEQ ID NO: 9 - protein, 7 aa, artificial sequence
  constructed sequence
IPISQGK

SEQ ID NO: 10 - protein, 9 aa, artificial sequence
  constructed sequence
ILNKPVGLK

SEQ ID NO: 11 - protein, 20 aa, artificial sequence
  constructed sequence
ELEVPVHTGPNSQKTADLTR

SEQ ID NO: 12 - protein, 8 aa, artificial sequence
  constructed sequence
GSRSQIPR

SEQ ID NO: 13 - protein, 8 aa, artificial sequence
  constructed sequence
ILSAQGCK

SEQ ID NO: 14 - protein, 9 aa, artificial sequence
  constructed sequence
NLSPGFNFR

SEQ ID NO: 15 - protein, 9 aa, artificial sequence
  constructed sequence
NYMMSNGYK

SEQ ID NO: 16 - protein, 15 aa, artificial sequence
  constructed sequence
FQGKWGTVCDDNFSK

SEQ ID NO: 17 - protein, 6 aa, artificial sequence
  constructed sequence
LFGGKK

SEQ ID NO: 18 - protein, 16 aa, artificial sequence
  constructed sequence
DIKPDNVLLDVNGHIR

SEQ ID NO: 19 - protein, 7 aa, artificial sequence
  constructed sequence
GALKQNK

SEQ ID NO: 20 - protein, 15 aa, artificial sequence
  constructed sequence
NSQGSEMFGDDDKRR

SEQ ID NO: 21 - protein, 9 aa, artificial sequence
  constructed sequence
KENSFEMQR

SEQ ID NO: 22 - protein, 13 aa, artificial sequence
  constructed sequence
RQAMKEMSIDQAR

SEQ ID NO: 23 - protein, 15 aa, artificial sequence
  constructed sequence
LLLSVLPQHVAMEMK

SEQ ID NO: 24 - protein, 8 aa, artificial sequence
  constructed sequence
TIELQMKK

SEQ ID NO: 25 - protein, 9 aa, artificial sequence
  constructed sequence
CSVNNQQSK

SEQ ID NO: 26 - protein, 14 aa, artificial sequence
  constructed sequence
NQELCQVAVEKSPK

SEQ ID NO: 27 - protein, 304 aa, artificial sequence
  constructed sequence
MSAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKLAA

SEQ ID NO: 28 - protein, 274 aa, artificial sequence
  constructed sequence
MASDVGGGRVTMAMVSMWCTSMNSAAVSHRCWADNSTAASGSSAASGNRHCRRRWDNATATSWSADTCVDGWVYDRSTSTVAKWNVCDSHAKMASYAGVGAAACGASDRWASARWTTGRDWGWRVAANGKGAVDTTVSAMRSMGASGTRMGRRTCSTCWAGTGADAGSNMGVVDAKMGASHGRRTAASAGCANTVHMGARSAAVGGGVGAATYTYSSTVRMTAVGGMAARGGAGVRGVHGWVYGTVVSGAATSDTDVNAVKKATHGTGNSVKST

SEQ ID NO: 29 - protein, 305 aa, artificial sequence
  constructed sequence
MSAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKLAAC

SEQ ID NO: 30 - DNA, 936 bp, artificial sequence
  constructed sequence
atgtcagcagtaaaggcagcaagatacggtaaagataatgtcagagtctacaaggttcacaaggacgaaaaaactggtgttcaaacagtttacgaaatgactgtttgtgttttgttggaaggtgaaatcgaaacttcttacacaaaggctgataactcagttattgttgcaacagattctattaaaaatactatctatatcacagctaagcaaaacccagttactccaccagaattgttcggttcaatcttgggtacacatttcatcgaaaagtacaaccatatccatgctgcacatgttaacatcgtttgtcatagatggactagaatggatattgatggtaaaccacatccacattcttttattagagattcagaagaaaagagaaatgttcaagttgatgttgttgagggtaaaggtatcgatatcaagtcttcattgtcaggtttaactgttttgaagtctacaaattcacaattttggggtttcttgagagatgaatacactacattgaaggaaacatgggatagaattttatctactgatgttgatgctacatggcaatggaagaacttctcaggtttgcaagaagttagatctcatgttccaaaatttgatgctacttgggctacagcaagagaagttactttgaagacattcgcagaagataactctgcttcagttcaagcaactatgtacaagatggctgaacaaatcttggcaagacaacaattgatcgaaacagttgaatattcattaccaaataagcattacttcgaaatcgatttgtcttggcataagggtttgcaaaacactggtaaaaatgctgaagttttcgcaccacaatctgatccaaatggtttgattaaatgcacagtcggtagatcctctttgaagtccaagttagcagcacaccatcatcatcaccattgctga

SEQ ID NO: 31 - protein, 311 aa, artificial sequence
  constructed sequence
MSAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKLAAHHHHHHC

SEQ ID NO: 32 - DNA, 918 bp, artificial sequence
  constructed sequence
atgtcagcagtaaaggcagcaagatacggtaaagataatgtcagagtctacaaggttcacaaggacgaaaaaactggtgttcaaacagtttacgaaatgactgtttgtgttttgttggaaggtgaaatcgaaacttcttacacaaaggctgataactcagttattgttgcaacagattctattaaaaatactatctatatcacagctaagcaaaacccagttactccaccagaattgttcggttcaatcttgggtacacatttcatcgaaaagtacaaccatatccatgctgcacatgttaacatcgtttgtcatagatggactagaatggatattgatggtaaaccacatccacattcttttattagagattcagaagaaaagagaaatgttcaagttgatgttgttgagggtaaaggtatcgatatcaagtcttcattgtcaggtttaactgttttgaagtctacaaattcacaattttggggtttcttgagagatgaatacactacattgaaggaaacatgggatagaattttatctactgatgttgatgctacatggcaatggaagaacttctcaggtttgcaagaagttagatctcatgttccaaaatttgatgctacttgggctacagcaagagaagttactttgaagacattcgcagaagataactctgcttcagttcaagcaactatgtacaagatggctgaacaaatcttggcaagacaacaattgatcgaaacagttgaatattcattaccaaataagcattacttcgaaatcgatttgtcttggcataagggtttgcaaaacactggtaaaaatgctgaagttttcgcaccacaatctgatccaaatggtttgattaaatgcacagtcggtagatcctctttgaagtccaagttagcagcatgctga

SEQ ID NO: 33 - protein, 311 aa, artificial sequence
  constructed sequence
MSAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKLAAGSGSGSC

SEQ ID NO: 34 - DNA, 936 bp, artificial sequence
  constructed sequence
atgtcagcagtaaaggcagcaagatacggtaaagataatgtcagagtctacaaggttcacaaggacgaaaaaactggtgttcaaacagtttacgaaatgactgtttgtgttttgttggaaggtgaaatcgaaacttcttacacaaaggctgataactcagttattgttgcaacagattctattaaaaatactatctatatcacagctaagcaaaacccagttactccaccagaattgttcggttcaatcttgggtacacatttcatcgaaaagtacaaccatatccatgctgcacatgttaacatcgtttgtcatagatggactagaatggatattgatggtaaaccacatccacattcttttattagagattcagaagaaaagagaaatgttcaagttgatgttgttgagggtaaaggtatcgatatcaagtcttcattgtcaggtttaactgttttgaagtctacaaattcacaattttggggtttcttgagagatgaatacactacattgaaggaaacatgggatagaattttatctactgatgttgatgctacatggcaatggaagaacttctcaggtttgcaagaagttagatctcatgttccaaaatttgatgctacttgggctacagcaagagaagttactttgaagacattcgcagaagataactctgcttcagttcaagcaactatgtacaagatggctgaacaaatcttggcaagacaacaattgatcgaaacagttgaatattcattaccaaataagcattacttcgaaatcgatttgtcttggcataagggtttgcaaaacactggtaaaaatgctgaagttttcgcaccacaatctgatccaaatggtttgattaaatgcacagtcggtagatcctctttgaagtccaagttagcagcaggttctggttctggttcttgctga

SEQ ID NO: 35 - protein, 254 aa, artificial sequence
  constructed sequence
MHHHHHHMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKAALPETGG

SEQ ID NO: 36 - DNA, 765 bp, artificial sequence
  constructed sequence
atgcaccaccaccaccaccacatggttagtaaaggggaagaattatttaccggcgtggtgccgattctggttgaactggacggcgacgtgaacggccacaaattcagcgttagcggcgagggcgaaggtgacgcgacctacggcaagctgaccctgaaatttatctgcaccaccggcaagctgccggtgccgtggccgaccctggttaccaccctgacctacggtgttcagtgcttcagccgttatccggaccacatgaagcaacacgatttctttaaaagcgcgatgccggagggctacgtgcaggaacgtaccatcttctttaaggacgatggtaactataaaacccgtgcggaagtgaagttcgaaggcgacaccctggttaaccgtatcgagctgaagggtattgactttaaagaagatggcaacattctgggtcacaaactggagtacaactataacagccacaacgtgtatatcatggcggataagcagaaaaacggcattaaggttaacttcaaaatccgtcacaacattgaagacggtagcgtgcaactggcggatcactaccagcaaaacaccccgattggcgacggtccggttctgctgccggataaccactatctgagcacccaaagcgcgctgagcaaggacccgaacgagaaacgtgatcacatggtgctgctggaatttgttaccgcggcgggtatcaccctgggtatggacgaactgtataaggcggcgctgccggagaccggcggttaa

SEQ ID NO: 37 - protein, 247 aa, artificial sequence
  constructed sequence
MHHHHHHMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKC

SEQ ID NO: 38 - DNA, 744 bp, artificial sequence
  constructed sequence
atgcaccaccaccaccaccacatggttagtaaaggggaagaattatttaccggcgtggtgccgattctggttgaactggacggcgacgtgaacggccacaaattcagcgttagcggcgagggcgaaggtgacgcgacctacggcaagctgaccctgaaatttatctgcaccaccggcaagctgccggtgccgtggccgaccctggttaccaccctgacctacggtgttcagtgcttcagccgttatccggaccacatgaagcaacacgatttctttaaaagcgcgatgccggagggctacgtgcaggaacgtaccatcttctttaaggacgatggtaactataaaacccgtgcggaagtgaagttcgaaggcgacaccctggttaaccgtatcgagctgaagggtattgactttaaagaagatggcaacattctgggtcacaaactggagtacaactataacagccacaacgtgtatatcatggcggataagcagaaaaacggcattaaggttaacttcaaaatccgtcacaacattgaagacggtagcgtgcaactggcggatcactaccagcaaaacaccccgattggcgacggtccggttctgctgccggataaccactatctgagcacccaaagcgcgctgagcaaggacccgaacgagaaacgtgatcacatggtgctgctggaatttgttaccgcggcgggtatcaccctgggtatggacgaactgtataagtgttaa

(19) United States
(12) Patent Application Publication
(10) Pub. No.: US 2024/0228983 A1
(43) Pub. Date: Jul. 11, 2024

(54) Modified Red Blood Cells and Uses Thereof For Treating Hyperuricemia and Gout

(71) Applicant: Westlake Therapeutics (Hangzhou) Co. Limited, Hangzhou, Zhejiang (CN)

(72) Inventors: Xiaofei Gao, Hangzhou, Zhejiang (CN); Xiaoqian Nie, Hangzhou, Zhejiang (CN); Jun Ren, Hangzhou, Zhejiang (CN); Yanjie Huang, Hangzhou, Zhejiang (CN); Xuan Liu, Hangzhou, Zhejiang (CN); Lu Liu, Hangzhou, Zhejiang (CN)

(21) Appl. No.: 18/264,098

(22) PCT Filed: Jan. 30, 2022
(86) PCT No.: PCT/CN2022/075140

§ 371 Date: Aug. 3, 2023

(30) Foreign Application Priority Data
Feb. 4, 2021 (WO) PCT/CN2021/075303

(51) Int. Cl. C12N 9/06; A61K 35/18; A61P 3/00; C12N 5/078; C12P 21/06

(52) U.S. Cl. CPC C12N 9/0048; A61K 35/18; A61P 3/00; C12N 5/0641; C12P 21/06

(57) ABSTRACT

Provided are a red blood cell (RBC) having an agent linked thereto, wherein the agent is linked to at least one endogenous, non-engineered membrane protein of the RBC by a sortase-mediated glycine conjugation or lysine side chain ε-amino group conjugation, and wherein the agent comprises a uric acid degrading polypeptide, a uric acid transporter or the combination; a method for preparing the RBC; and the use of the RBC for preventing or treating a disorder, condition or disease associated with an elevated uric acid level including hyperuricemia or gout.

### TECHNICAL FIELD

The present disclosure relates generally to modified red blood cells (RBCs), and more particularly to covalently modified RBCs and use of the same for treating hyperuricemia and gout.

### BACKGROUND

Gout is the most common form of inflammatory arthritis in adults, especially in men, with a prevalence ranging from 1% to 4% globally. Gout occurs when monosodium urate crystal (MSU) deposited in tissues, causing inflammation and intense pain of a gout attack. The biologic precursor to gout is elevated serum uric acid (UA) levels (i.e., hyperuricemia). Although hyperuricemia is the strongest single risk factor for the development of gout and is universally present in patients with gout, not all individuals with hyperuricemia develop gout. Recent work has emphasized the importance of the innate immune response.

Conventional urate-lowering agents such as anti-inflammatory drugs (colchicine), xanthine oxidase inhibitors (allopurinol, febuxostat) or uricosuric agents (probenecid, benzbromarone) induce very slow reduction in UA deposits, not allowing for the rapid resolution of tophi for all patients with gout and are mainly used at the early stage.

Urate oxidase (UOX, uricase) is a liver enzyme that metabolizes UA into allantoin, a more water-soluble compound, which is easily excreted by the kidney. All mammals produce UOX, except humans and certain primates. Indeed, during evolution, UOX was inactivated in humans primarily due to missense and frame-shift mutations in the gene encoding this enzyme. Uricase undeniably represents a valuable treatment option for chronic tophaceous gout when conventional urate-lowering agents may not be used.

Rasburicase, a recombinant UOX from A.flavus, was approved by EM EA in 2001 (Fasturtec®) and the Food and Drug Administration (FDA) in 2002 (Elitek®) for tumor lysis syndrome. This agent significantly reduces serum UA levels and acts faster than allopurinol. The recommended dose is 0.2 mg/kg in children and adults. However, its biological half-life is short (only 21 h), so rasburicase is given by infusion once daily for ≤7 days. In addition, recent studies have shown that repeated UOX injections could cause anaphylactic reactions with the production of antibodies that neutralize UOX enzyme activity [ref 11-16].

Pegloticase, a recombinant porcine UOX, with a baboon C-terminal sequence, is a modified pegylated recombinant UOX developed to be the first non-immunogenic biologic for treating the hyperuricemia of refractory gout, approved by the FDA for patients with chronic gout. A 6-month study versus placebo showed that pegloticase (infused at 8 mg every 2 weeks), induced a significant decrease in plasma UA in about 40% of the patients (associated with a tendency for tophi dissolution). However, the remaining patients had no response, which was correlated with the formation of pegloticase antibodies and infusion reactions. More than 10% of the patients treated with pegloticase had adverse events such as kidney stones, joint pain, anaemia, muscle spasms, dyspnea, headache, nausea, and fever [ref 17-21].

Available recombinant UOX (rasburicase, pegloticase) drugs are potent hypouricemic agents for gout. However, there are several limitations for the current therapy. First, UOX has significant immunogenicity and it may induce severe allergic reactions. Conjugating therapeutic enzymes to PEG may reduce immune responses in patients. However, studies showed that many patients treated with PEG-conjugated enzymes developed anti-PEG antibodies. Moreover, PEG may adversely affect the activity of the conjugated enzyme, leading to reduced efficacy in the treatment. Second, the therapeutic enzymes may become inactivated or eliminated in vivo due to short half-life, limited bioavailability, and/or interactions with plasma proteins. Third, production and purification of the enzymes tend to be time-consuming, and thus treatments with enzyme replacement therapy are very costly. The cost of treatment (calculated on an annual basis) is estimated to be about € 7200 for rasburicase and E 41,240 for pegloticase. Therefore, we need new gout therapy that is more efficacious and safer.

Urate oxidase from A. flavus is a 135 kDa homo-tetrameric enzyme. Each monomer is composed of two structurally equivalent tunneling-fold or T-fold motifs, which comprise an antiparallel four-stranded β-sheet with a pair of antiparallel α-helices layered on the concave side of the sheet. The concatenation of the two T-fold motifs gives rise to an antiparallel β-sheet of eight sequential strands, and the side-by-side dimer thus consists of an α8β16 barrel with the eight helices forming the exterior of the barrel. The active tetramer is then formed by dimer of dimer in a head-to-head arrangement, with an external size of 50 Å×50 Å and an internal tunnel of 50 Å long and 12 Å in diameter.

RBCs have been developed as drug delivery carriers by direct encapsulation, noncovalent attachment of foreign peptides, or through installation of proteins by fusion to antibodies specific for RBC surface proteins. It has been demonstrated that such modified RBCs have limitations for applications in vivo. For instance, encapsulation will disrupt cell membranes which subsequently affect in vivo survival rates of engineered cells. In addition, the non-covalent attachment of polymeric particles to RBCs dissociates readily, and the payloads will be degraded shortly in vivo.

Bacterial sortases are transpeptidases capable of modifying proteins in a covalent and site-specific manner. Wild type sortase A from Staphylococcus aureus (wt SrtA) recognizes an LPXTG motif and cleaves between threonine and glycine to form a covalent acyl-enzyme intermediate between the enzyme and the substrate protein. This intermediate is resolved by a nucleophilic attack by a peptide or protein normally with three consecutive glycine residues (3×glycines, G3) at the N-terminus. Previous studies have genetically overexpressed a membrane protein KELL with LPXTG motif on its C-terminus on RBCs, which can be attached to the N terminus of 3×glycines- or G(n=3)-modified proteins/peptides by using wt SrtA. These RBCs carrying drugs have shown efficacy in treating diseases on animal models. However, this requires steps of engineering hematopoietic stem or progenitor cells (HSPCs) and differentiating these cells into mature RBCs, which significantly limits the application.

Accordingly, there remains a need in the art for improved strategy for treating hyperuricemia and in particular gout.

### SUMMARY

In one general aspect, provided is a red blood cell (RBC) having an agent linked thereto, wherein the agent is linked to at least one endogenous, non-engineered membrane protein of the RBC by a sortase-mediated reaction, preferably by a sortase-mediated glycine conjugation and/or a sortase-mediated lysine side chain ε-amino group conjugation, and wherein the agent comprises a uric acid degrading polypeptide.

In some embodiments, the sortase-mediated glycine conjugation and/or the sortase-mediated lysine side chain ε-amino group conjugation occur at least on glycine(n) and/or lysine ε-amino group at internal sites of the extracellular domain of the at least one endogenous, non-engineered membrane protein, preferably n being 1 or 2.

In some embodiments, the RBC has not been genetically engineered to express a protein comprising a sortase recognition motif or a nucleophilic acceptor sequence, and preferably the RBC is a natural RBC such as a natural human RBC.

In some embodiments, the sortase is capable of mediating a glycine(n) conjugation and/or a lysine side chain ε-amino group conjugation, preferably at internal sites of the extracellular domain of the at least one endogenous, non-engineered membrane protein, preferably n being 1 or 2.

In some embodiments, the sortase is a Sortase A (SrtA) such as a Staphylococcus aureus transpeptidase A variant (mgSrtA).

In some embodiments, the mgSrtA comprises or consists essentially of or consists of an amino acid sequence having at least 60% identity to an amino acid sequence as set forth in SEQ ID NO: 3.

In some embodiments, the agent, before being linked to the RBC, comprises a sortase recognition motif on its C-terminus.

In some embodiments, the agent comprises a structure of (A1-Sp)ₘ-M, in which A1 represents the agent, Sp represents the optional spacers, and M represents the sortase recognition motif; m being an integer greater than or equal to 1, preferably m=1 to 3.

In some embodiments, the sortase recognition motif comprises or consists essentially of or consists of an amino acid sequence selecting from a group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS and LPXTA, wherein X is any amino acid.

In some embodiments, the sortase recognition motif comprises an unnatural amino acid located at position 5 from the direction of N-terminal to C-terminal of the sortase recognition motif, wherein the unnatural amino acid is an optionally substituted hydroxyl carboxylic acid having a formulae of CH₂OH—(CH₂)n-COOH, n being an integer from 0 to 3, preferably n=0.

In some embodiments, the sortase recognition motif comprises or consists essentially of or consists of an amino acid sequence selecting from a group consisting of LPXT*Y, LPXA*Y, LPXS*Y, LPXL*Y, LPXV*Y, LGXT*Y, LAXT*Y, LSXT*Y, NPXT*Y, MPXT*Y, IPXT*Y, SPXT*Y, VPXT*Y and YPXR*Y, wherein * represents the optionally substituted hydroxyl carboxylic acid; and X and Y independently represent any amino acid.

In some embodiments, the sortase recognition motif comprises or consists essentially of or consists of an amino acid sequence selecting from a group consisting of LPXT*G, LPXA*G, LPXS*G, LPXL*G, LPXV*G, LGXT*G, LAXT*G, LSXT*G, NPXT*G, MPXT*G, IPXT*G, SPXT*G, VPXT*G, YPXR*G, LPXT*S and LPXT*A, preferably the sortase recognition motif is LPET*G with * being 2-hydroxyacetic acid.

In some embodiments, the agent linked to the at least one endogenous, non-engineered membrane protein on the surface of the BRC comprises a structure of (A1-Sp)ₘ-L1-P1, in which L1 is linked to a glycine(n) in P1, and/or a structure of (A1-Sp)ₘ-L1-P2, in which L1 is linked to the side chain ε-amino group of lysine in P2, wherein n is preferably 1 or 2, A1 represents the agent, Sp represents the optional spacers, L1 is selected from the group consisting of LPXT, LPXA, LPXS, LPXL, LPXV, LGXT, LAXT, LSXT, NPXT, MPXT, IPXT, SPXT, VPXT, and YPXR, P1 and P2 independently represent the extracellular domain of the at least one endogenous, non-engineered membrane protein, and X represents any amino acids; m being an integer greater than or equal to 1, preferably m=1 to 3.

In some embodiments, the Sp is selected from a group consisting of the following types: (1) zero-length type. (2) amine-sulfhydryl type; (3) homobifunctional NHS esters type; (4) homobifunctional imidoesters type; (5) carbonyl-sulfydryl type; (6) sulfhydryl reactive type; and (7) sulfhydryl-hydroxy type; and preferably the one or more Sp is an NHS ester-maleimide heterobifunctional crosslinker such as 6-Maleimidohexanoic acid and 4-Maleimidobutyric acid and the agent comprises an exposed sulfydryl, preferably an exposed cysteine, more preferably a terminal cysteine, most preferably a C-terminal cysteine.

In some embodiments, the uric acid degrading polypeptide comprises one or more polypeptides selected from a group consisting of: uricase, HIU hydrolase, OHCU decarboxylase, allantoinase and allantoicase, preferably uricase comprising an amino acid sequence set forth in SEQ ID NO: 27 or a functional variant or fragment thereof.

In some embodiments, the agent additionally comprises a uric acid transporter, which preferably comprises one or more polypeptides selected from a group consisting of: URAT1, GLUT9, OAT4, OAT1, OAT3, Gal-9, ABCG2, SLC34A2, MRP4, OAT2, NPT1, NPT4, and MCT9, preferably URAT1 comprising an amino acid sequence set forth in SEQ ID NO: 28 or a functional variant or fragment thereof.

In another aspect, provided is a composition comprising a plurality of the red blood cells as described herein and a physiologically acceptable carrier.

In another aspect, provided is a method for preparing the red blood cell of as described herein, comprising contacting a red blood cell (RBC) with a sortase substrate that comprises a sortase recognition motif and an agent, in the presence of a sortase under conditions suitable for the sortase to conjugate the sortase substrate to the at least one endogenous, non-engineered membrane protein of the RBC by a sortase-mediated reaction, preferably by a sortase-mediated glycine conjugation and/or a sortase-mediated lysine side chain ε-amino group conjugation, wherein the agent comprises a uric acid degrading polypeptide, or a combination of a uric acid degrading polypeptide and a uric acid transporter.

In some embodiments, the sortase substrate comprises a structure of (A1-Sp)ₘ-M, in which A1 represents an agent, Sp represents the optional spacers, and M represents a sortase recognition motif; m being an integer greater than or equal to 1, preferably m=1 to 3.

In some embodiments, the sortase recognition motif comprises or consists essentially of or consists of an amino acid sequence selecting from a group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS and LPXTA, wherein X is any amino acid.

In some embodiments, the sortase recognition motif comprises an unnatural amino acid located at position 5 from the direction of N-terminal to C-terminal of the sortase recognition motif, wherein the unnatural amino acid is an optionally substituted hydroxyl carboxylic acid having a formulae of CH₂OH—(CH₂)n-COOH, n being an integer from 0 to 3, preferably n=0.

In some embodiments, the sortase recognition motif comprises or consists essentially of or consists of an amino acid sequence selecting from a group consisting of LPXT*Y, LPXA*Y, LPXS*Y, LPXL*Y, LPXV*Y, LGXT*Y, LAXT*Y, LSXT*Y, NPXT*Y, MPXT*Y, IPXT*Y, SPXT*Y, VPXT*Y and YPXR*Y, wherein * represents the optionally substituted hydroxyl carboxylic acid; and X and Y independently represent any amino acid.

In some embodiments, the sortase recognition motif comprises or consists essentially of or consists of an amino acid sequence selecting from a group consisting of LPXT*G, LPXA*G, LPXS*G, LPXL*G, LPXV*G, LGXT*G, LAXT*G, LSXT*G, NPXT*G, MPXT*G, IPXT*G, SPXT*G, VPXT*G, YPXR*G, LPXT*S and LPXT*A, preferably the sortase recognition motif is LPET*G with * being 2-hydroxyacetic acid.

In some embodiments, the Sp is selected from a group consisting of the following types: (1) zero-length type; (2) amine-sulfhydryl type; (3) homobifunctional NHS esters type; (4) homobifunctional imidoesters type; (5) carbonyl-sulfydryl type; (6) sulfhydryl reactive type; and (7) sulfhydryl-hydroxy type; and preferably the one or more Sp is an NHS ester-maleimide heterobifunctional crosslinker such as 6-Maleimidohexanoic acid and 4-Malcimidobutyric acid and the agent comprises an exposed sulfydryl, preferably an exposed cysteine, more preferably a terminal cysteine, most preferably a C-terminal cysteine.

In another aspect, provided is a method for treating or preventing a disorder, condition or disease associated with an elevated uric acid level in a subject in need thereof, comprising administering the red blood cell or the composition of as described herein to the subject.

In some embodiments, the subject has a serum uric acid level greater than about 8.0 mg/dl prior to the administering.

In some embodiments, the disorder, condition or disease associated with an elevated uric acid level is selected from a group consisting of hyperuricemia, gout (e.g., chronic refractory gout, gout tophus and gouty arthritis), metabolic syndrome, tumor lysis syndrome, Lesch-Nyhan syndrome, cardiovascular disease, diabetes, hypertension, renal disease, and uric acid nephrolithiasis.

In another aspect, provided is use of the red blood cell or the composition as described herein in the manufacture of a medicament for treating or preventing a disorder, condition or disease associated with an elevated uric acid level in a subject in need thereof.

In some embodiments, the subject has a serum uric acid level greater than about 8.0 mg/dl prior to the administering.

In some embodiments, the disorder, condition or disease associated with an elevated uric acid level is selected from a group consisting of hyperuricemia, gout (e.g., chronic refractory gout, gout tophus and gouty arthritis), metabolic syndrome, tumor lysis syndrome, Lesch-Nyhan syndrome, cardiovascular disease, diabetes, hypertension, renal disease, and uric acid nephrolithiasis.

In another aspect, provided is a red blood cell or the composition as described herein for use in treating or preventing a disorder, condition or disease associated with an elevated uric acid level in a subject in need thereof, preferably being selected from a group consisting of hyperuricemia, gout (e.g., chronic refractory gout, gout tophus and gouty arthritis), metabolic syndrome, tumor lysis syndrome, Lesch-Nyhan syndrome, cardiovascular disease, diabetes, hypertension, renal disease, and uric acid nephrolithiasis.

### BRIEF DESCRIPTION OF THE DRAWINGS

In the drawings, embodiments of the present disclosure are illustrated by way of example. It is to be expressly understood that the description and drawings are only for the purpose of illustration and as an aid to understanding, and are not intended as a definition of the limits of the invention.

FIGS. 1A-1K show efficient labeling of peptides and proteins on the surface of natural mouse or human RBCs by wild type sortase (wt SrtA or wtSrtA) and mutant sortase (mg SrtA or mgSrtA).

FIGS. 1A and 1B. 10⁹/mL mouse (FIG. 1A) or human (FIG. 1B) RBCs were incubated with 500 μM biotin-LPETG with or without 40 μM wild type (wt) SrtA or mg SrtA for 2 hrs at 4° C. After the enzymatic reaction, the labeling efficacy was detected by incubating RBCs with PE-conjugated streptavidin and analyzed by flow cytometry. Histograms show biotin signals on the surface of RBCs labeled with or without mg or wt sortase.

FIG. 1C. 10⁹/mL of mouse RBCs were incubated with 8 μM biotin-LPETG peptides and 40 μM mg or wt SrtA for 2 hrs at 37° C. The labeling efficacy was analyzed by immunoblotting with Streptavidin-HRP. Hemoglobin Subunit Alpha 1, HBA1, was used as the loading control.

FIG. 1D. 10⁹/mL of mouse RBCs were processed for the enrichment of membrane proteins by ultracentrifugation. Significant enrichment of membrane proteins was detected by Western-blotting of an RBC membrane protein Band 3 encoded by Slc4a1 gene.

FIG. 1E. 10⁹/mL of mouse RBCs were biotin-labeled by mg SrtA and subjected to the membrane protein enrichment. Western-blot results showed a significant increase in biotin signals after the enrichment step compared to that of unenriched samples.

FIG. 1F. 10⁹ mouse RBCs were sortagged with biotin-LPETG by mg SrtA or wt SrtA. After sortagging, labeled RBCs were stained with DiR dye and injected intravenously into the mice. Mice were bled at 24 h post transfusion. Blood samples were incubated with FITC-conjugated Streptavidin at 37° C. for 1 hour for the detection of biotin signals and washed three times before being analyzed by flow cytometry. DiR positive cells were selected for analyzing the percentage of RBCs with biotin signals.

FIG. 1G. Mice were bled at indicated days post transfusion. DiR positive cells indicate the percentage of transfused RBCs in the circulation.

FIG. 1H. DiR positive RBCs from the blood samples of the above experiments were analyzed for the percentage of biotin positive cells.

FIG. 1I. At day 4 post injection, blood samples were analyzed by imaging flow cytometry for the sortagging of biotin on RBCs. Blood samples were incubated with FITC-conjugated Streptavidin at 37° C. for 1 hour for the detection of biotin signals and washed three times before being analyzed by flow cytometry.

FIG. 1J. 10⁹/mL mouse RBCs were sortagged with 100 μM eGFP-LPETG by mg SrtA or wt SrtA at 37° C. for 2 h. The efficacy of conjugation was analyzed by flow cytometry. Histograms show biotin signals on the surface of RBCs labeled with or without mg or wt sortase. Red: no sortase; blue: mg sortase; orange: wt sortase.

FIG. 1K. 10⁹ eGFP-sortagged mouse RBCs were stained by DiR dye and injected intravenously into the mice. At day 7 post injection, the mice were bled and the blood samples were analyzed by imaging flow cytometry for eGFP signals on the surface of RBCs.

FIG. 2 shows intravenous injection of OT-1-RBCs induces immunotolerance in OT-1 TCR T cells in vivo.

FIG. 2A. 10⁶ CD8⁺ T cells purified from CD45.1 OT-1 TCR transgenic mice were intravenously injected into CD45.2 recipient mice. After 24 hrs, 2×10⁹ mouse RBCs were labeled with or without OT-1 peptides mediated by mg SrtA and transfused into the recipient mice, which will be challenged with OT-1 peptide with complete freund's adjuvant (CFA). At day 15, these mice were euthanized and subjected to spleen harvest.

FIG. 2B. Suspended cells isolated from spleen were analyzed by flow cytometry. CD8⁺ T cells were first selected out for analyzing the percentage of CD45.1+ T cells, which demonstrates the survival of adoptively transferred OT-1 TCR CD8+ T cells. CD45.1+CD8+ T cells were further analyzed for the expression of PD1 and CD44. CD45.2: membrane protein expressed on the surface of many hematopoietic cells used for indicating endogenous T cells in this experiment. CD44: marker for T cell activation; PD-1: marker for cell apoptosis and exhaustion.

FIG. 3 shows chemical structure of irreversible linker 6-Mal-LPET*G (6-Maleimidohexanoic acid-Leu-Pro-Glu-Thr-2-hydroxyacetic acid-Gly; 6-Mal represents 6-Maleimidohexanoic acid).

FIG. 4 shows reaction scheme for conjugation of irreversible linker 6-Mal-LPET*G to a modified protein. The two reaction substrates are mixed and reacted in a ratio of 1:4=eGFP-cys:6-Mal-LPET*G to obtain the final reaction product.

FIG. 5 shows chemical structure of irreversible linker 6-Mal-K(6-Mal)-GGG-K(6-Mal)-GGGSAA-LPET*G and 6-Mal-K(6-Mal)-GGGGGGSAA-LPET*G (top) and schematic diagram of protein conjugated by double fork and triple fork (bottom).

FIG. 6 shows product identified by mass spectrometry. Chromatographic desalt and separate protein, then the protein samples were analyzed on a 6230 TOF LC/MS spectrometer. Entropy incorporated in BioConfirm 10.0 software.

FIG. 7 shows eGFP-cys protein sequence and detection results of protein side chain modification by tandem mass spectrometry.

FIG. 8 shows efficient labeling of eGFP-cys-6-Mal-LPET*G on the surface of natural RBCs by the mutant sortase (mgSrtA). RBCs were incubated with 75 μM eGFP-cys-6-Mal-LPET*G with 10 μM mg SrtA for 2 hrs at 37° C. After the enzymatic reaction, the labeling efficacy was detected by flow cytometry. Histograms show eGPF signals on the surface. Red: Unlabeled; blue: eGFP-LPETG; orange: eGFP-cys-6-Mal-LPET*G.

FIG. 9 shows the results of 10⁹ mouse RBCs that were sortagged with eGFP-cys-6-Mal-LPET*G by mg SrtA. After sortagging, labeled RBCs were stained with DiR dye and injected intravenously into the mice. Mice were bled at 24 h post transfusion. Blood samples analyzed by flow cytometry. DiR positive cells were selected for analyzing the percentage of RBCs with eGFP signals.

FIG. 10 shows the percentage of transfused RBCs in the circulation as indicated by DiR positive cells. Mice were bled at indicated days post transfusion.

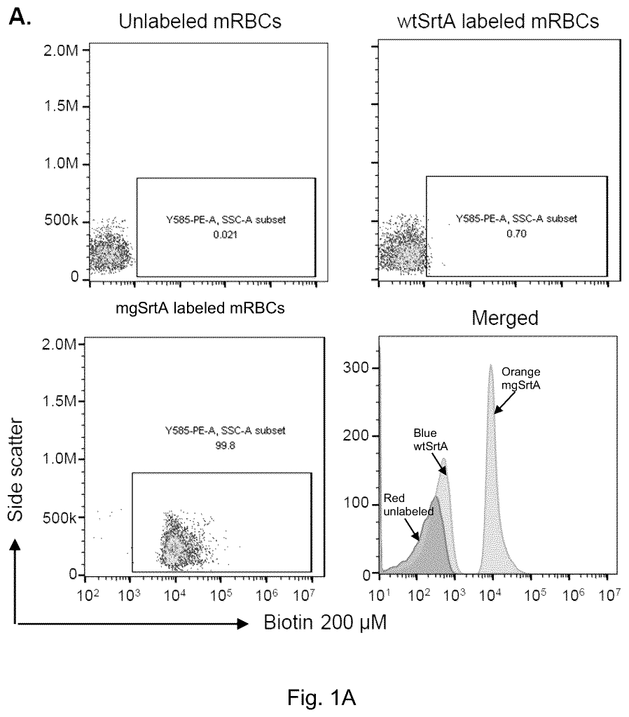

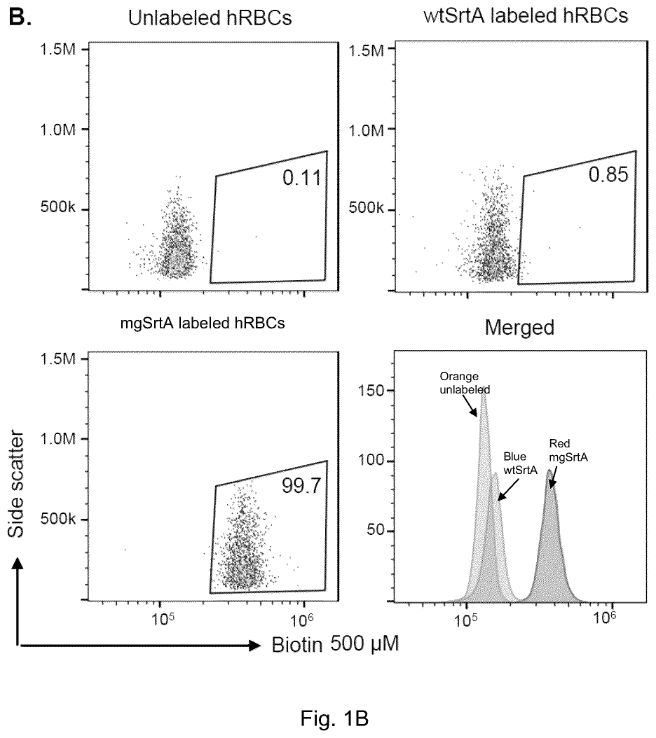

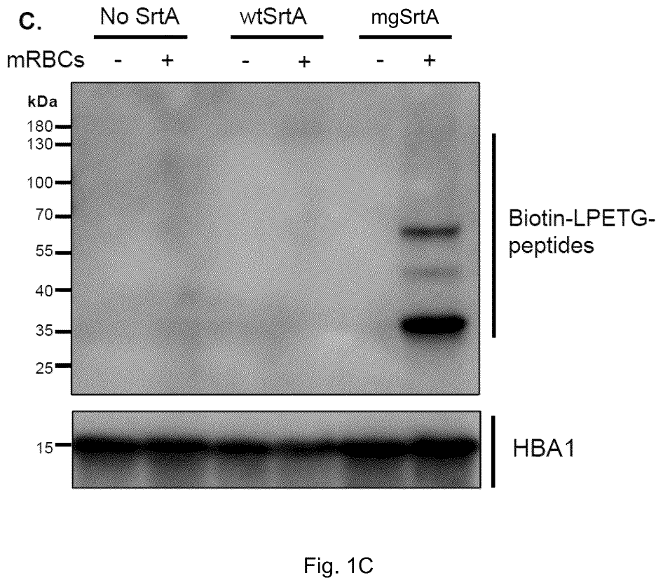

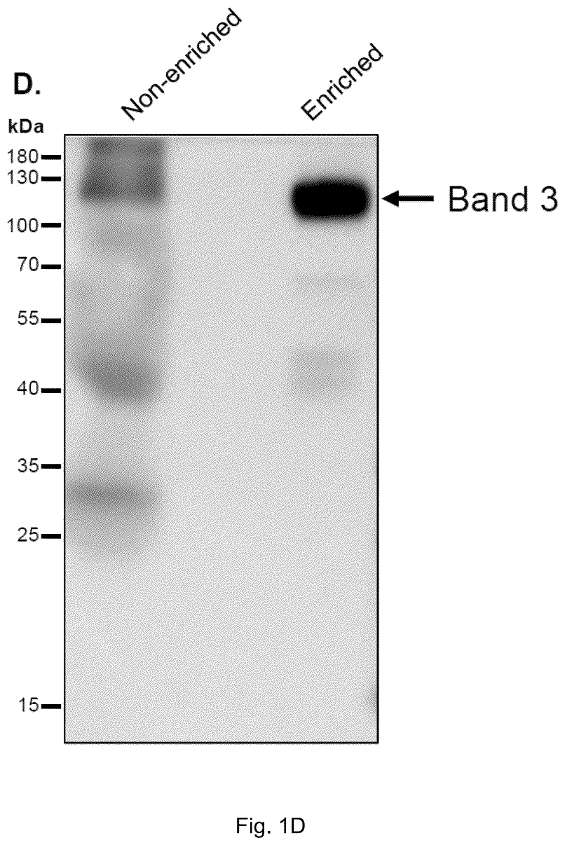

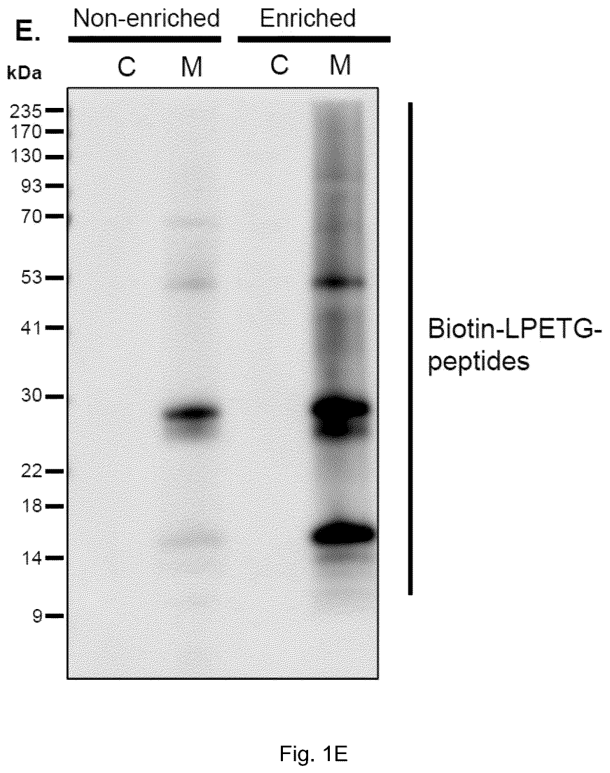

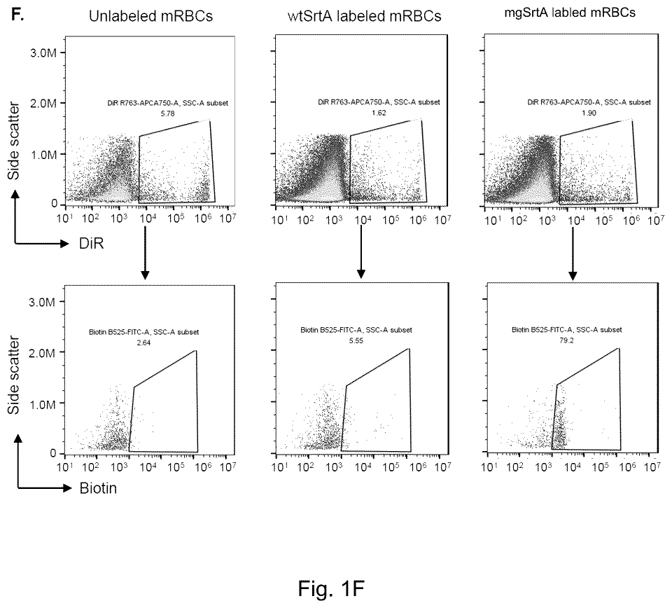

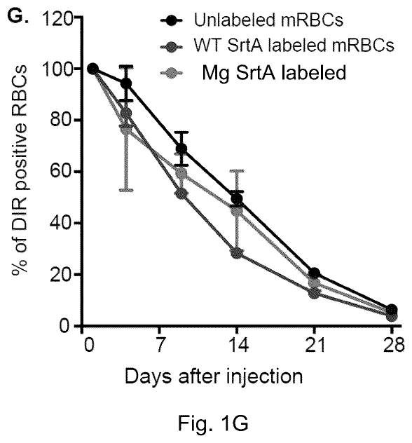

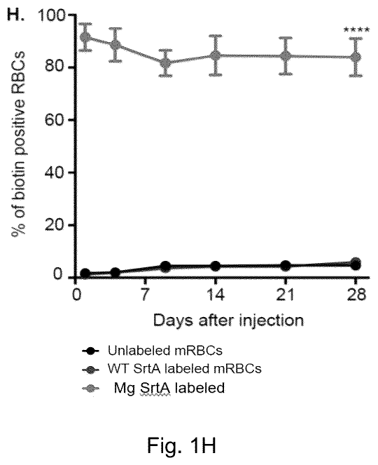

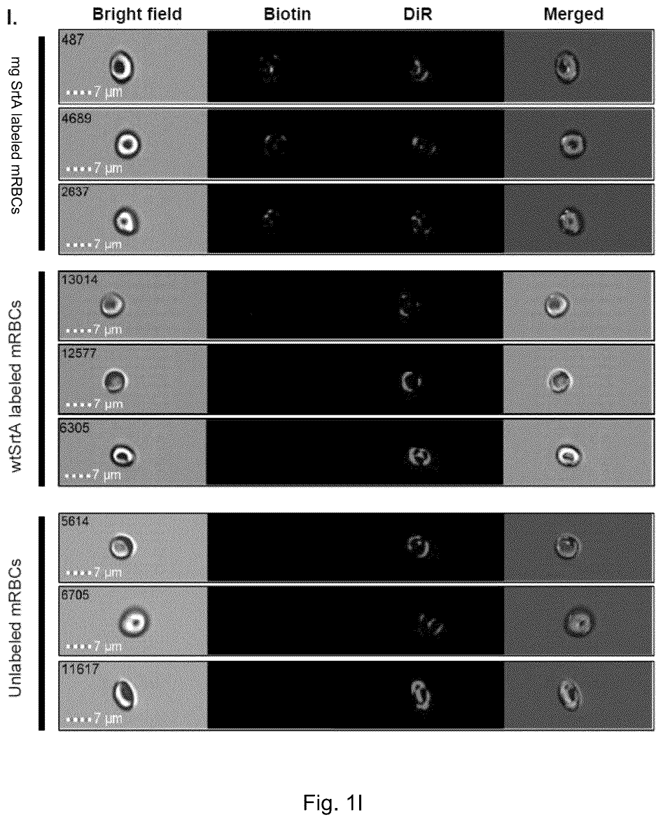

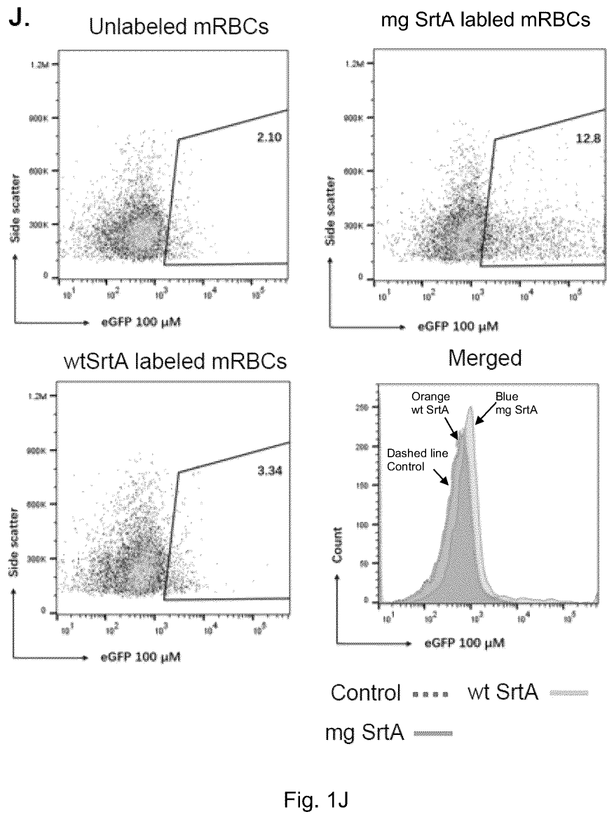

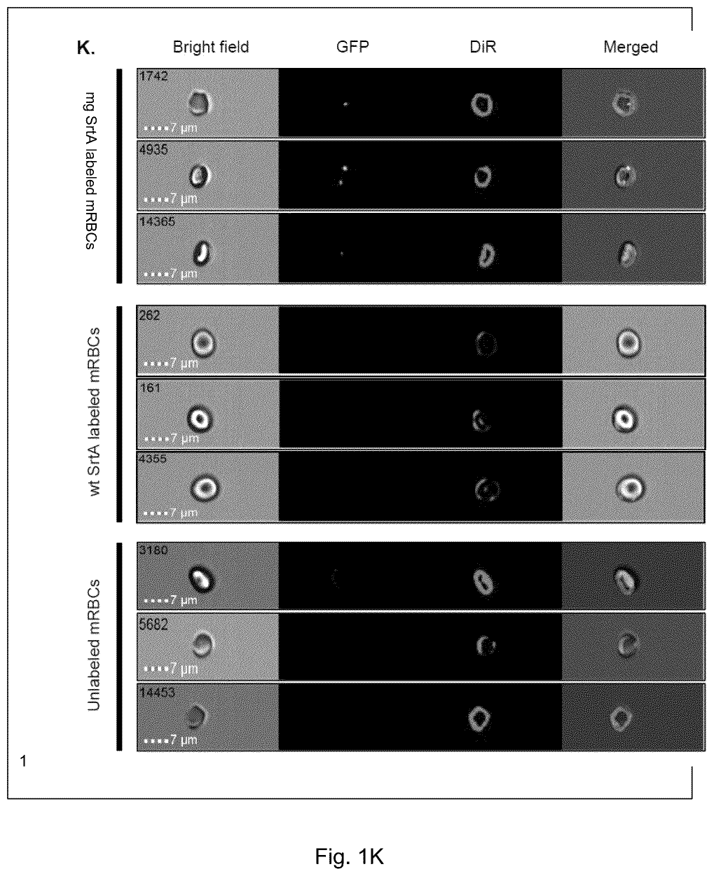

FIG. 1 shows the percentage of eGFP positive cells obtained by analyzing DiR positive RBCs from the blood samples of the above experiments.

FIG. 12 shows imaging analysis of eGFP signals on the cell surface. 10⁹ eGFP-sortagged mouse RBCs were stained by DiR dye and injected intravenously into the mice. At day 7 post injection, the mice were bled and the blood samples were analyzed by imaging flow cytometry for eGFP signals on the surface of RBCs.

FIG. 13 shows the labeling efficiency of UOX-His₆-Cys-LPET*G on the surface of natural RBCs by mg SrtA. Histograms showed His tag signals on the surface of RBCs labeled with mg sortase (UOX-RBCs) or without mg sortase (control) (FIG. 13A: mouse RBCs; FIG. 13B: human RBCs; FIG. 13C: rat RBCs; FIG. 13D: cynomolgus monkeys RBCs).

FIG. 14 shows the functional analysis of engineered red blood cells in vitro. The uric acid concentration was evaluated at the specified time to determine the consumption rates of UA by the engineered RBCs of rat (FIG. 14A) and cynomolgus monkey (FIG. 14B) respectively in vitro.

FIG. 15 shows the survival and stability of the engineered red blood cells in vivo. Control RBCs and UOX-RBCs were reacted with Far red and transfused into cynomolgus monkeys via i.v. injection. RBC in vivo survival was tracked via Far red fluorescence using flow cytometry.

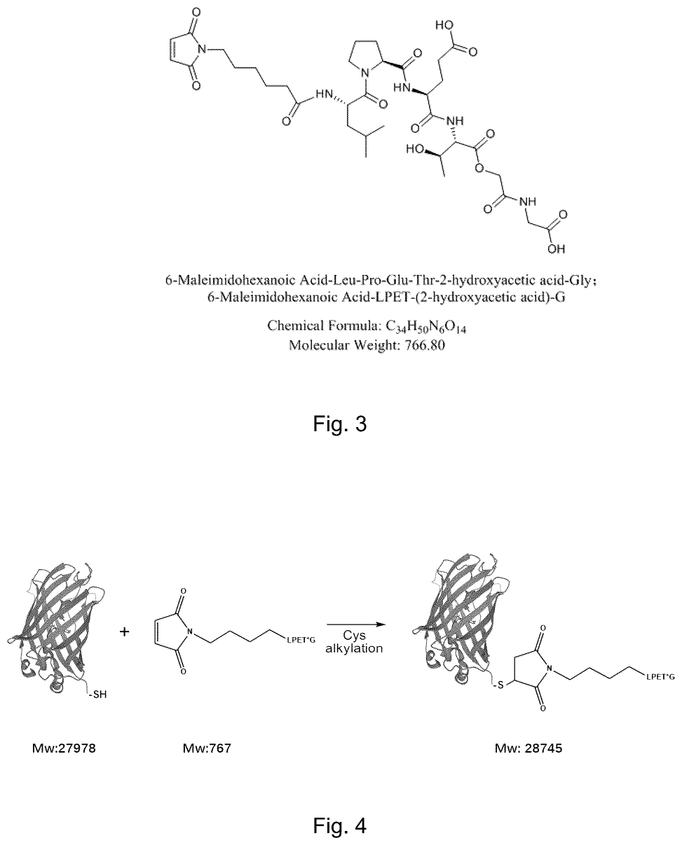

FIG. 16 depicts the representative PET images of radiolabeled UOX-RBCs in cynomolgus monkeys. Cynomolgus monkeys were intravenously injected with radiolabeled UOX-RBCs. PET scans were acquired at 0.5 h (FIG. 16A), 1 h (FIG. 16B) and 3 h (FIG. 16C) following the transfusion. PET showed the most significant distribution of UOX-RBCs in the liver and spleen, while accumulation was lower in heart, brain and muscle.

FIG. 17 shows the functional analysis of engineered red blood cells in vivo. Rat UOX-RBCs reduced UA concentration in the rat hyperuricemia model following repeated transfusion (FIG. 17A: 1^{st} transfusion; FIG. 17B: 2^{nd} transfusion; FIG. 17C: 3^{rd} transfusion).

FIG. 18 shows anti-UOX IgG antibody titres in Cynomolgus monkeys (FIG. 18A) and rats (FIG. 18B).

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, unless otherwise specified, the scientific and technical terms used herein have the meanings as generally understood by a person skilled in the art. Although any methods and materials similar or equivalent to those described herein find use in the practice of the present invention, the preferred methods and materials are described herein. Accordingly, the terms defined herein are more fully described by reference to the Specification as a whole.

As used herein, the singular terms “a,” “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skills in the art.

As used herein, the term “consisting essentially of” in the context of an amino acid sequence is meant the recited amino acid sequence together with additional one, two, three, four or five amino acids at the N- or C-terminus.

Unless the context requires otherwise, the terms “comprise”, “comprises” and “comprising”, or similar terms are intended to mean a non-exclusive inclusion, such that a recited list of elements or features does not include those stated or listed elements solely, but may include other elements or features that are not listed or stated.

As used herein, the terms “patient”, “individual” and “subject” are used in the context of any mammalian recipient of a treatment or composition disclosed herein. Accordingly, the methods and composition disclosed herein may have medical and/or veterinary applications. In a preferred form, the mammal is a human.

As used herein, the term “sequence identity” is meant to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, California, USA).

Recent studies have discovered mutant sortases with different specificities in motif recognition[4]. For instance, Ge et al. showed that an evolved SrtA variant (mg SrtA) is capable of recognizing the N-terminus of Gi-modified peptide, which cannot be achieved by wt SrtA[5]. In addition, membrane proteins with a single glycine at the N-terminus are much more abundant than those with 3×glycines. Ge et al. made an N-terminal sequence analysis of human membrane proteome with a predicted N-terminal glycine(s). The list of 182 proteins that contain N-terminal glycine residues after enzymatic removal of the signal peptide or the initiator methionine residue according to the previous study[7]. Among them, 176 proteins (96.70%) contain a single glycine residue at the N-terminus, 4 proteins (2.20%) contain a GG residue at the N-terminus, while only 2 proteins (1.10%) contain a G_{(n≥3)} residue at the N-terminus. None of the 182 proteins is known to be expressed on the surface of mature human red blood cells.

Herein, the present disclosure is at least partially based on a surprising finding that in spite of the absence of known N-terminal glycine(s), it is possible to conjugate a sortase substrate to at least one endogenous, non-engineered membrane protein of natural human RBC by a sortase-mediated glycine conjugation and/or a sortase-mediated lysine side chain conjugation occurring at least on glycine_{(n=1 or 2)} and lysine ε-amino group at internal sites of the extracellular domain of the at least one endogenous, non-engineered membrane protein. Without being limited by theory, it is contemplated that a non-canonical function of sortase enables conjugation of a sortase substrate to internal glycines_{(n=1 or 2)} and/or lysine side chain ε-amino group in the extracellular domain of endogenous, non-engineered membrane protein. Also, without being limited by any theory, extensive tissue-specific mRNA splicing and protein translation during erythropoiesis might lead to exposure of glycine_{(n=1 or 2)}.

The inventors therefore develop a new strategy to covalently modify endogenous, non-engineered membrane proteins of natural RBCs with peptides and/or small molecules through a sortase-mediated reaction. The technology allows for producing RBC products by directly modifying natural RBCs instead of HSPCs which are limited by their resources. Also, the modified RBCs preserve their original biological properties well and remain stable as their native state.

In order to more effectively increase the yield of the product and reduce the occurrence of reverse reactions, the inventors of the present disclosure further surprisingly found that modifying proteins by chemical coupling can greatly reduce the protein concentration required during a cell labeling process.

To achieve RBC labeling by sortase, a cysteine residue was incorporated in the C-terminal position of each monomer of UOX to facilitate maleimide-mediated LPETG peptide conjugation. The side-by-side configuration of the dimer causes that the first β-strand of one monomer is aligned adjacent to the last β-strand of the other, and the N-terminal region of one monomer is in close proximity with the C-terminal tail of the neighboring monomer. Due to these unique structural characteristics, the N-terminal residues of each monomer should remain unchanged or slightly truncated to avoid excess residues from hindering sortase binding to LPETG peptide. Moreover, His₆ tag can be inserted between the C-terminal of the monomer and the cysteine residue when IMAC is used as a recombinant protein purification strategy, and incorporation of a spacer like the purification tag or GS linker of equivalent length at this position also maintains the enzyme in a sufficient distance from the sortase binding site, which may be favored in consideration of the steric effect. If was found that the strategy for labeling as described herein can label natural red blood cells at a very high efficiency and maintain the enzyme activity of a uric acid degrading polypeptide (e.g. UOX) in vitro and in vivo and the RBCs labeled with a uric acid degrading polypeptide (e.g. UOX) can successfully reduce blood uric acid level in vivo without significant adverse effects, as shown by the no change of haematology, coagulation, blood biochemistry and urinalysis that can be attributed to the administered UOX-RBCs.

### Red Blood Cells (RBCs)

In some aspects, the present disclosure provides a red blood cell (RBC) having an agent linked thereto, wherein the agent is linked to at least one endogenous, non-engineered membrane protein of the RBC by a sortase-mediated reaction. In some embodiments, the agent is linked to at least one endogenous, non-engineered membrane protein through a sortase-mediated glycine conjugation and/or a sortase-mediated lysine side chain ε-amino conjugation. In some embodiments, the sortase-mediated glycine conjugation and/or the sortase-mediated lysine side chain ε-amino group conjugation occur at least on glycine₍ₙ₎ and/or lysine ε-amino group in the extracellular domain (for example at internal sites of the extracellular domain or the N-terminal of the extracellular domain) of the at least one endogenous, non-engineered membrane protein, preferably n being 1 or 2. In some embodiments, without being limited to any theory, the sortase-mediated glycine conjugation may occur at exposed glycine_{(n=1 or 2)} of previously unreported membrane proteins due to tissue-specific mRNA splicing and protein translation during erythropoiesis. In some embodiments, the exposed glycine_{(n=1 or 2)} may be N-terminal exposed glycine_{(n=1 or 2)}. In some embodiments, the sortase-mediated lysine side chain ε-amino group conjugation occurs at ε-amino group of terminal lysine or internal lysine of the extracellular domain. In some embodiments, the sortase-mediated glycine conjugation and/or the sortase-mediated lysine side chain ε-amino group conjugation may occur at glycine(n) and/or lysine ε-amino group at terminal (e.g., N-terminal) and/or internal sites of the extracellular domain of at least one endogenous, non-engineered membrane protein, preferably n being 1 or 2.

Unless otherwise indicated or clearly evident from the context, where the present disclosure refers to a red blood cell (RBC), it is generally intended to mean a mature red blood cell. In certain embodiments, the RBC is a human RBC, such as a human natural RBC.

In some embodiments, the RBC is a red blood cell that has not been genetically engineered to express a protein comprising a sortase recognition motif or a nucleophilic acceptor sequence. In some embodiments the RBC has not been genetically engineered. Unless otherwise indicated or clearly evident from the context, where the present disclosure refers to sortagging red blood cells it is generally intended to mean red blood cells that have not been genetically engineered for sortagging. In certain embodiments the red blood cells are not genetically engineered.

A red blood cell is considered “not genetically engineered for sortagging” if the cell has not been genetically engineered to express a protein comprising a sortase recognition motif or a nucleophilic acceptor sequence in a sortase-catalyzed reaction.

In some embodiments, the present disclosure provides red blood cells having an agent conjugated thereto via a sortase-mediated reaction. In some embodiments, a composition comprising a plurality of such cells is provided. In some embodiments, at least a selected percentage of the cells in the composition are modified, i.e., having an agent conjugated thereto by sortase. For example, in some embodiments at least 5%, 10%, 15%, 20%. 25%, 30%, 40%, 50%, 60%, 70%, 80%. 90%, 95%, 96%, 97%, 98%, 99%, or more of the cells have an agent conjugated thereto. In some embodiments, the conjugated agent may be one or more of the agents described herein. In some embodiments, the agent may be conjugated to glycine₍ₙ₎ and/or lysine ε-amino group in one or more or all of the sequences as listed in Table 5 (e.g., SEQ ID NOs: 5-26). In some embodiments, the agent may be conjugated to glycine₍ₙ₎ and/or lysine ε-amino group in a sequence comprising SEQ ID NO: 5.

In some embodiments, the present disclosure provides a red blood cell that comprises an agent conjugated via a sortase-mediated reaction to a non-genetically engineered endogenous polypeptide expressed by the cell. In some embodiments, two, three, four, five or more different endogenous non-engineered polypeptides expressed by the cell have an agent conjugated thereto via a sortase-mediated reaction. The agents attached to different polypeptides may be the same or the cell may be sortagged with a plurality of different agents.

In some embodiments, the present disclosure provides a red blood cell (RBC) having an agent linked via a sortase mediated reaction to a glycine₍ₙ₎ or a side chain of lysine located anywhere (preferably internal sites) in an extracellular domain of at least one endogenous, non-engineered membrane protein on the surface of the BRC, wherein n is preferably 1 or 2. In some embodiments, the agent is linked to one or more (e.g., two, three, four or five) glycine₍ₙ₎ or lysine side chain ε-amino groups in or within the extracellular domain. In certain embodiment, the at least one endogenous, non-engineered membrane protein may be selected from a group consisting of the membrane proteins listed in Table 5 below or any combination thereof. In certain embodiment, the at least one endogenous non-engineered membrane protein may be selected from a group consisting of the 22 membrane proteins listed in Table 5 or any combination thereof. In some embodiments, the sortase-mediated glycine conjugation and/or the sortase-mediated lysine side chain ε-amino group conjugation may occur at glycine₍ₙ₎ and/or lysine ε-amino group in one or more or all of the sequences as listed in Table 5 (e.g., SEQ ID NOs: 5-26). In certain embodiments, the at least one endogenous non-engineered membrane protein may comprise extracellular calcium-sensing receptor (CaSR) (a parathyroid cell calcium-sensing receptor, PCaR1). In certain embodiments, the linking may be one or more or all of the modifications as shown in Table 5 below. In certain embodiments, the linking may occur on one or more positions selected from the modification positions as listed in Table 5 and any combination thereof, e.g., positions comprising G526 and/or K527 positions of CaSR; G158 and/or K162 of CD antigen CD3g; and/or G950 and/or K964 of TrpC2.

In some embodiments, without being limited to any theory, the agent may be linked to a protein selected from a group consisting of proteins listed in Tables 2, 3 and/or 4 below or any combination thereof.

In some embodiments, the present disclosure provides a red blood cell (RBC) having an agent linked to at least one endogenous, non-engineered membrane protein on the surface of the BRC. In some embodiments, the agent is linked via a sortase recognition motif to the at least one endogenous, non-engineered membrane protein. In some embodiments, the sortase recognition motif may be selected from a group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS and LPXTA, wherein X is any amino acid. In some embodiments, the sortase recognition motif may comprise an unnatural amino acid located at position 5 from the direction of N-terminal to C-terminal of the sortase recognition motif, wherein the unnatural amino acid is an optionally substituted hydroxyl carboxylic acid having a formulae of CH₂OH—(CH₂)ₙ—COOH, n being an integer from 0 to 3, preferably n=0. In some embodiments, the sortase recognition motif comprising an unnatural amino acid may be selected from a group consisting of LPXT*Y, LPXA*Y, LPXS*Y, LPXL*Y, LPXV*Y, LGXT*Y, LAXT*Y, LSXT*Y, NPXT*Y, MPXT*Y, IPXT*Y, SPXT*Y, VPXT*Y and YPXR*Y, wherein * represents the optionally substituted hydroxyl carboxylic acid; and X and Y independently represent any amino acid. In some embodiments, the sortase recognition motif comprising a unnatural amino acid may be selected from a group consisting of LPXT*G, LPXA*G, LPXS*G, LPXL*G, LPXV*G, LGXT*G, LAXT*G, LSXT*G, NPXT*G, MPXT*G, IPXT*G, SPXT*G, VPXT*G, YPXR*G, LPXT*S and LPXT*A, preferably M is LPET*G with * preferably being 2-hydroxyacetic acid.

It can be understood that after the agent linked to the membrane protein, the last one or two residues from 5^{th} position (from the direction of N-terminal to C-terminal) of the sortase recognition motif is replaced by the amino acid on which the linkage occurs, as described elsewhere herein. For example, the agent linked to the at least one endogenous, non-engineered membrane protein comprises (A¹-Sp)ₘ-L¹-P¹, in which L¹ is linked to a glycine(˜) in P¹, and/or a structure of (A-Sp)ₘ-L¹-P², in which L¹ is linked to the side chain ε-amino group of lysine in P², wherein n is preferably 1 or 2; L¹ is selected from the group consisting of LPXT, LPXA, LPXS, LPXL, LPXV, LGXT, LAXT, LSXT, NPXT, MPXT, IPXT, SPXT, VPXT and YPXR; A¹ represents the agent; Sp represents the optional spacers; m being an integer greater than or equal to 1, preferably m=1 to 3; P¹ and P² independently represent the at least one endogenous, non-engineered membrane protein; and X represents any amino acids. In some embodiments, the agent linked to the at least one endogenous, non-engineered membrane protein comprises (A¹-Sp)ₘ-LPXT-P¹, in which LPXT is linked to a glycine₍ₙ₎ in P¹, and/or a structure of (A-Sp)ₘ-LPXT-P², in which LPXT is linked to the side chain ε-amino group of lysine in P², wherein n is preferably 1 or 2, A¹ represents the agent, Sp represents the spacers, m being an integer greater than or equal to 1, preferably m=1 to 3; P¹ and P² independently represent the at least one endogenous, non-engineered membrane protein, and X represents any amino acids. In some embodiments, P¹ and P² may be the same or different. In some embodiments, the agent is linked to one or more (e.g., two, three, four, five or more) glycine₍ₙ₎ or lysine side chain ε-amino groups in or within an extracellular domain of the at least one endogenous, non-engineered membrane protein. In certain embodiment, the at least one endogenous, non-engineered membrane protein may be selected from a group consisting of the membrane proteins listed in Table 5 below or any combination thereof. In certain embodiment, the at least one endogenous non-engineered membrane protein may be selected from a group consisting of the 22 membrane proteins listed in Table 5 or any combination thereof. In some embodiments, the sortase-mediated glycine conjugation and/or the sortase-mediated lysine side chain ε-amino group conjugation may occur at glycine₍ₙ₎ and/or lysine ε-amino group in one or more or all of the sequences as listed in Table 5 (e.g., SEQ ID NOs: 5-26). In certain embodiments, at least one endogenous non-engineered membrane protein may comprise extracellular calcium-sensing receptor (CaSR) (a parathyroid cell calcium-sensing receptor, PCaR1). In certain embodiments, the linking may be one or more or all of the modifications as shown in Table 5 below. In certain embodiments, the linking may occur on one or more positions selected from the modification positions as listed in Table 5 and any combination thereof, e.g., positions comprising G526 and/or K527 positions of CaSR; G158 and/or K162 of CD antigen CD3g; and/or G950 and/or K964 of TrpC2.

In some embodiments, genetically engineered red blood cells are modified by using sortase to attach a sortase substrate to a non-genetically engineered endogenous polypeptide of the cell. The red blood cell may, for example, have been genetically engineered to express any of a wide variety of products, e.g., polypeptides or noncoding RNAs, may be genetically engineered to have a deletion of at least a portion of one or more genes, and/or may be genetically engineered to have one or more precise alterations in the sequence of one or more endogenous genes. In certain embodiments, a non-engineered endogenous polypeptide of such genetically engineered cell is sortagged with any of the various agents described herein.

In some embodiments, the present disclosure contemplates using autologous red blood cells that are isolated from an individual to whom such isolated red blood cells, after modified in vitro, are to be administered. In some embodiments, the present disclosure contemplates using immuno-compatible red blood cells that are of the same blood group as an individual to whom such cells are to be administered (e.g., at least with respect to the ABO blood type system and, in some embodiments, with respect to the D blood group system) or may be of a compatible blood group.

### Endogenous, Non-Engineered Membrane Proteins

The terms “non-engineered, “non-genetically modified” and “non-recombinant” as used herein are interchangeable and refer to not being genetically engineered, absence of genetic modification, etc. Non-engineered membrane proteins encompass endogenous proteins. In certain embodiments, a non-genetically engineered red blood cell does not contain a non-endogenous nucleic acid, e.g., DNA or RNA that originates from a vector, from a different species, or that comprises an artificial sequence, e.g., DNA or RNA that was introduced artificially. In certain embodiments, a non-engineered cell has not been intentionally contacted with a nucleic acid that is capable of causing a heritable genetic alteration under conditions suitable for uptake of the nucleic acid by the cells.

In some embodiments, the endogenous non-engineered membrane proteins may encompass any or at least one of the membrane proteins listed in Table 5 below or any combination thereof. In certain embodiments, the endogenous non-engineered membrane proteins may encompass any or at least one of the 22 membrane proteins listed in Table 5 or any combination thereof. In certain embodiments, the endogenous non-engineered membrane proteins may encompass extracellular calcium-sensing receptor (CaSR) (a parathyroid cell calcium-sensing receptor, PCaR1).

### Sortase

Enzymes identified as “sortases” have been isolated from a variety of Gram-positive bacteria. Sortases, sortase-mediated transacylation reactions, and their use in protein engineering are well known to those of ordinary skills in the art (see, e.g., PCT/US2010/000274 (WO/2010/087994), and PCT/US2011/033303 (WO/2011/133704)). Sortases have been classified into 4 classes, designated A, B, C, and D, based on sequence alignment and phylogenetic analysis of 61 sortases from Gram-positive bacterial genomes (Dramsi S, Trieu-Cuot P, Bierne H, Sorting sortases: a nomenclature proposal for the various sortases of Gram-positive bacteria. Res Microbiol. 156(3):289-97, 2005). Those skilled in the art can readily assign a sortase to the correct class based on its sequence and/or other characteristics such as those described in Drami, et al., supra. The term “sortase A” as used herein refers to a class A sortase, usually named SrtA in any particular bacterial species, e.g., SrtA from S. aureus or S. pyogenes.

The term “sortase” also known as transamidases refers to an enzyme that has transamidase activity. Sortases recognize substrates comprising a sortase recognition motif, e.g., the amino acid sequence LPXTG. A molecule recognized by a sortase (i.e., comprising a sortase recognition motif) is sometimes termed a “sortase substrate” herein. Sortases tolerate a wide variety of moieties in proximity to the cleavage site, thus allowing for the versatile conjugation of diverse entities so long as the substrate contains a suitably exposed sortase recognition motif and a suitable nucleophile is available. The terms “sortase-mediated transacylation reaction”, “sortase-catalyzed transacylation reaction”, “sortase-mediated reaction”, “sortase-catalyzed reaction”, “sortase reaction”, “sortase-mediated transpeptide reaction” and like terms, are used interchangeably herein to refer to such a reaction. The terms “sortase recognition motif”, “sortase recognition sequence” and “transamidase recognition sequence” with respect to sequences recognized by a transamidase or sortase, are used interchangeably herein. The term “nucleophilic acceptor sequence” refers to an amino acid sequence capable of serving as a nucleophile in a sortase-catalyzed reaction, e.g., a sequence comprising an N-terminal glycine (e.g., 1, 2, 3, 4, or 5 N-terminal glycines) or in some embodiments comprising internal glycines_{(n=1 or 2)} or lysine side chain ε-amino group.

The present disclosure encompasses embodiments relating to any of the sortase classes known in the art (e.g., a sortase A, B, C or D from any bacterial species or strain). In some embodiments, sortase A is used, such as SrtA from S. aureus. In some embodiments it is contemplated to use two or more sortases. In some embodiments the sortases may utilize different sortase recognition sequences and/or different nucleophilic acceptor sequences.

In some embodiments, the sortase is a sortase A (SrtA). SrtA recognizes the motif LPXTG, with common recognition motifs being, e.g., LPKTG, LPATG, LPNTG. In some embodiments LPETG is used. However, motifs falling outside this consensus may also be recognized. For example, in some embodiments the motif comprises an ‘A’, ‘S’, ‘L’ or ‘V’ rather than a ‘T’ at position 4, e.g., LPXAG, LPXSG, LPXLG or LPXVG, e.g., LPNAG or LPESG, LPELG or LPEVG. In some embodiments the motif comprises an ‘A’ rather than a ‘G’ at position 5, e.g., LPXTA, e.g., LPNTA. In some embodiments the motif comprises a ‘G’ or ‘A’ rather than ‘P’ at position 2, e.g., LGXTG or LAXTG, e.g., LGATG or LAETG. In some embodiments the motif comprises an ‘I’ or ‘M’ rather than ‘L’ at position 1, e.g., MPXTG or IPXTG, e.g., MPKTG, IPKTG, IPNTG or IPETG. Diverse recognition motifs of sortase A are described in Pishesha et al. 2018.

In some embodiments, the sortase recognition sequence is LPXTG, wherein X is a standard or non-standard amino acid. In some embodiments, X is selected from D, E, A, N, Q, K, or R. In some embodiments, the recognition sequence is selected from LPXTG, LPXAG, LPXSG, LPXLG, LPXVG, LGXTG, LAXTG, LSXTG, NPXTG, MPXTG, IPXTG, SPXTG, VPXTG, YPXRG, LPXTS and LPXTA, wherein X may be any amino acids, such as those selected from D, E, A, N, Q, K, or R in certain embodiments.

In some embodiments, the sortase may recognizes a motif comprising an unnatural amino acid, preferably located at position 5 from the direction of N-terminal to C-terminal of the sortase recognition motif. The unnatural amino acid is a substituted or unsubstituted hydroxyl carboxylic acid having a formulae of CH₂OH—(CH₂)ₙ—COOH, n being an integer from 0 to 5, e.g., 0, 1, 2, 3, 4 and 5, preferably n=0. In some embodiments, the unnatural amino acid is a substituted hydroxyl carboxylic acid and in some further embodiments, the hydroxyl carboxylic acid is substituted by one or more substituents selected from halo, C₁₋₆ alkyl, C_{J-6} haloalkyl, hydroxyl, C₁₋₆ alkoxy, and C₁₋₆ haloalkoxy. The term “halo” or “halogen” means fluoro, chloro, bromo, or iodo, and preferred are fluoro and chloro. The term “alkyl” by itself or as part of another substituent refers to a hydrocarbyl radical of Formula CₙH₂ₙ₊₁ wherein n is a number greater than or equal to 1. In some embodiments, alkyl groups useful in the present disclosure comprise from 1 to 6 carbon atoms, preferably from 1 to 4 carbon atoms, more preferably from 1 to 3 carbon atoms, still more preferably 1 to 2 carbon atoms. Alkyl groups may be linear or branched and may be further substituted as indicated herein. C_{x-y} alkyl refers to alkyl groups which comprise from x to y carbon atoms. Suitable alkyl groups include methyl, ethyl, n-propyl, i-propyl, n-butyl, i-butyl, s-butyl and tert-butyl, pentyl and its isomers (e.g. n-pentyl, iso-pentyl), and hexyl and its isomers (e.g. n-hexyl, iso-hexyl). Preferred alkyl groups include methyl, ethyl, n-propyl, i-propyl, n-butyl, i-butyl, s-butyl and tert-butyl. The term “haloalkyl” alone or in combination, refers to an alkyl radical having the meaning as defined above, wherein one or more hydrogens are replaced with a halogen as defined above. Non-limiting examples of such haloalkyl radicals include chloromethyl, 1-bromoethyl, fluoromethyl, difluoromethyl, trifluoromethyl, 1,1,1-trifluoroethyl and the like.

In some embodiments, the sortase recognition motif comprising an unnatural amino acid may be selected from a group consisting of LPXT*Y, LPXA*Y, LPXS*Y, LPXL*Y, LPXV*Y, LGXT*Y, LAXT*Y, LSXT*Y, NPXT*Y, MPXT*Y, IPXT*Y, SPXT*Y, VPXT*Y and YPXR*Y, wherein * represents the optionally substituted hydroxyl carboxylic acid; and X and Y independently represent any amino acid. In some embodiments, the sortase recognition motif comprising a unnatural amino acid may be selected from a group consisting of LPXT*G, LPXA*G, LPXS*G, LPXL*G, LPXV*G, LGXT*G, LAXT*G, LSXT*G, NPXT*G, MPXT*G, IPXT*G, SPXT*G, VPXT*G, YPXR*G, LPXT*S and LPXT*A, preferably M is LPET*G with * preferably being 2-hydroxyacetic acid.

In some embodiments, the present disclosure contemplates using a variant of a naturally occurring sortase. In some embodiments, the variant is capable of mediating a glycine(f) conjugation and/or a lysine side chain ε-amino group conjugation, preferably at internal sites of the extracellular domain of the at least one endogenous, non-engineered membrane protein of a red blood cell, preferably n being 1 or 2. Such variants may be produced through processes such as directed evolution, site-specific modification, etc. Considerable structural information regarding sortase enzymes, e.g., sortase A enzymes, is available, including NMR or crystal structures of SrtA alone or bound to a sortase recognition sequence (see, e.g., Zong Y, et al. J. Biol Chem. 2004, 279, 31383-31389). The active site and substrate binding pocket of S. aureus SrtA have been identified. One of ordinary skills in the art can generate functional variants by, for example, avoiding deletions or substitutions that would disrupt or substantially alter the active site or substrate binding pocket of a sortase. In some embodiments, directed evolution on SrtA can be performed by utilizing the FRET (Fluorescence Resonance Energy Transfer)-based selection assay described in Chen, et al. Sci. Rep. 2016, 6 (1), 31899. In some embodiments, a functional variant of S. aureus SrtA may be those described in CN106191015 Å and CN109797194A. In some embodiments, the S. aureus SrtA variant can be a truncated variant with e.g. 25-60 (e.g., 30, 35, 40, 45, 50, 55, 59 or 60) amino acids being removed from N-terminus.

In some embodiments, a functional variant of S. aureus SrtA useful in the present disclosure may be a S. aureus SrtA variant comprising one or more mutations on amino acid positions of D124, Y187, E189 and F200 of D124G, Y187L, E189R and F200L and optionally further comprising one or more mutations of P94S/R, D160N, D165A, K190E and K196T. In certain embodiments, the S. aureus SrtA variant may comprise D124G; DI24G and F200L; P94S/R, D124G, D160N, D165A, K190E and K196T; P94S/R, D160N, D165A, Y187L, E189R, K190E and K196T; P94S/R, D124G, D160N, D165A, Y187L, E189R, K190E and K196T; D124G, Y187L, E189R and F200L; or P94S/R, D124G, D160N, D165A, Y187L, E189R, K190E, K196T and F200L. In some embodiments, the S. aureus SrtA variants have 59 or 60 (e.g., 25, 30, 35, 40, 45, 50, 55, 59 or 60) amino acids being removed from N-terminus. In some embodiments, the mutated amino acid positions above are numbered according to the numbering of a wild type S. aureus SrtA, e.g., as shown in SEQ ID NO: 1. In some embodiments, the full length nucleotide sequence of the wild type S. aureus SrtA is shown as in e.g., SEQ ID NO: 2.

(full length, GenBank Accession No.:
CAA3829591.1)
SEQ ID NO: 1

| | |
|---|---|
| 1 | MKKWINRLMT IAGVVLILVA AYLFSKPHID NYLADKDKDE |
| | KIEQYDKNVK |
| 51 | EQASKDKKQQ AKPQIPKDKS KVAGYIEIPD ADIKEPVYPG |
| | PATPEQLNRG |
| 101 | VSFAEENESL DDQNISIAGH TFIDRPNYQF TNLKAAKKGS |
| | MVYFKVGNET |
| 151 | RKYKMTSIRD VKPTDVGVLD EQKGKDKOLT LITCDDYNEK |
| | TGVWEKRKIF |
| 201 | VATEVK |

(full length, wild type)
SEQ ID NO: 2
ATGAAAAAATGGACAAATCGATTAATGACAATCGCTGGTGTGGTACTTAT
CCTAGTGGCAGCATATTTGTTTGCTAAACCACATATCGATAATTATCTTC
ACGATAAAGATAAAGATGAAAAGATTGAACAATATGATAAAAATGTAAAA
GAACAGGCGAGTAAAGATAAAAAGCAGCAAGCTAAACCTCAAATTCCGAA
AGATAAATCGAAAGTGGCAGGCTATATTGAAATTCCAGATGCTGATATTA
AAGAACCAGTATATCCAGGACCAGCAACACCTGAACAATTAAATAGAGGT
GTAAGCTTTGCAGAAGAAAATGAATCACTAGATGATCAAAATATTTCAAT
TGCAGGACACACTTTCATTGACCGTCCGAACTATCAATTTACAAATCTTA
AAGCAGCCAAAAAAGGTAGTATGGTGTACTTTAAAGTTGGTAATGAAACA
CGTAAGTATAAAATGACAAGTATAAGAGATGTTAAGCCTACAGATGTAGG
AGTTCTAGATGAACAAAAAGGTAAAGATAAACAATTAACATTAATTACTT
GTGATGATTACAATGAAAAGACAGGCGTTTGGGAAAAACGTAAAATCTTT
GTAGCTACAGAAGTCAAA

In some embodiments, as compared to a wild type S. aureus SrtA, the S. aureus SrtA variant may comprise one or more mutations at one or more of the positions corresponding to 94, 105, 108, 124, 160, 165, 187, 189, 190, 196 and 200 of SEQ ID NO: 1. In some embodiments, as compared to a wild type S. aureus SrtA, the S. aureus SrtA variant may comprise one or more mutations corresponding to P94S/R, E105K, E108A, D124G, D160N, D165A, Y187L, E189R, K190E, K196T and F200L. In some embodiments, as compared to a wild type S. aureus SrtA, the S. aureus SrtA variant may comprise one or more mutations corresponding to D124G, Y187L, E189R and F200L and optionally further comprises one or more mutations corresponding to P94S/R, D160N, D165A, K190E and K196T and optionally further one or more mutations corresponding to E105K and E108A. In certain embodiments, as compared to a wild type S. aureus SrtA, the S. aureus SrtA variant may comprise mutations corresponding to D124G; D124G and F200L; P94S/R, DI24G, D160N, D165A, K190E and K196T; P94S/R, D160N, D165A, Y187L, E189R, K190E and K196T; P94S/R, D124G, D160N, D165A, Y187L, E189R, K190E and K196T; D124G, Y187L, E189R and F200L; or P94S/R, D124G, D160N, D165A, Y187L, E189R, K190E, K196T and F200L. In some embodiments, the S. aureus SrtA variant may comprise one or more mutations of P94S/R, E105K, E108A, D124G, D160N, D165A, Y187L. E189R, K190E, K196T and F200L relative to SEQ ID NO: 1. In some embodiments, the S. aureus SrtA variant may comprise D124G, Y187L, E189R and F200L and optionally further comprises one or more mutations of P94S/R, D160N, D165A, K190E and K196T and optionally further comprises E105K and/or E108A relative to SEQ ID NO: 1. In certain embodiments, the S. aureus SrtA variant may, comprise, relative to SEQ ID NO: 1, D124G; D124G and F200L; P94S/R, D124G, D160N, D165A, K190E and K196T; P94S/R, D160N, D165A, Y187L, E189R, K190E and K196T; P94S/R, D124G, D160N, D165A, Y187L, E189R, K190E and K196T; D124G, Y187L, E189R and F200L; or P94S/R, D124G, D160N, D165A, Y187L, E189R, K190E, K196T and F200L. In some embodiments, mutations E105K and/or E108A/Q allows the sortase-mediated reaction to be Ca²⁺ independent. In some embodiments, the S. aureus SrtA variants as described herein may have 25-60 (e.g., 25, 30, 35, 40, 45, 50, 55, 56, 57, 58, 59, or 60) amino acids being removed from N-terminus. In some embodiments, the mutated amino acid positions above are numbered according to the numbering of a full length of a wild type S. aureus SrtA, e.g., as shown in SEQ ID NO: 1.

In some embodiments, a functional variant of S. aureus SrtA useful in the present disclosure may be a S. aureus SrtA variant comprising one or more mutations of P94S/R, E105K, E108A/Q, D124G, D160N, D165A, Y187L, E189R, K190E, K196T and F200L. In certain embodiments, the S. aureus SrtA variant may comprise P94S/R, E105K, E108Q, D124G, D160N, D165A, Y187L, E189R, K190E, K196T and F200L; or P94S/R, E105K, E108A, D124G, D160N, D165A, Y187L, E189R, K190E, K196T and F200L. In some embodiments, the S. aureus SrtA variant may comprise one or more mutations of P94S/R, E105K, E108A/Q, D124G, D160N, D165A, Y187L, E189R, K190E, K196T and F200L relative to SEQ ID NO: 1. In certain embodiments, the S. aureus SrtA variant may comprise P94S/R, E105K, E108Q, D124G, D160N, D165A, Y187L, E189R, K190E, K196T and F200L relative to SEQ ID NO: 1; or P94S/R, E105K, E108A, D124G, D160N, D165A, Y187L, E189R, K190E, K196T and F200L relative to SEQ ID NO: 1. In some embodiments, the S. aureus SrtA variants have 25-60 (e.g., 25, 30, 35, 40, 45, 50, 55, 56, 57, 58, 59, or 60) amino acids being removed from N-terminus. In some embodiments, the mutated amino acid positions above are numbered according to the numbering of a wild type S. aureus SrtA, e.g., as shown in SEQ ID NO: 1.

In some embodiments, the present disclosure contemplates a S. aureus SrtA variant (mg SrtA) comprising or consisting essentially of or consisting of an amino acid sequence having at least 60% (e.g., at least 65%, 70%, 75%, 80%, 85%, 90%, 91%, 92%, 93%, 94%, 95%, 96%, 97%, 98%, 99%, 99.5%, 99.9% or higher) identity to an amino acid sequence as set forth in SEQ ID NO: 3. In some embodiments, SEQ ID NO: 3 is a truncated SrtA and the mutations corresponding to wild type SrtA are shown in bold and underlined below. In some embodiments, the SrtA variant comprises or consists essentially of or consists of an amino acid sequence having at least 60% (e.g., at least 65%, 70%, 75%, 80%, 85%, 90%, 91%, 92%, 93%, 94%, 95%, 96%, 97%, 98%, 99%, 99.5%, 99.9% or higher) identity to an amino acid sequence as set forth in SEQ ID NO: 3 and comprises the mutations of P94R/S, D124G, D160N, D165A, Y187L, E189R, K190E, K196T and F200L and optionally E105K and/or E108A/Q (numbered according to the numbering of SEQ ID NO: 1).

(mutations shown in bold and underlined)
SEQ ID NO: 3

| | |
|---|---|
| 1 | KPHIDNYLHD KDKDEKIEQY DKNVKEQASK DKKQQAKPQI |
| | PKDKSKVAGY |
| 51 | IEIPDADIKE PVYPGPATRE QLNRGVSFAE ENESLDDQNI |
| | SIAGHTFIGR |
| 101 | PNYQFTNLKA AKKGSMVYFK VGNETRKYKM TSIRNVKPTA |
| | VGVLDEQKGK |
| 151 | DKOLTLITCD DLNRETGVWE TRKILVATEV K |

In some embodiments, the present disclosure provides a nucleic acid encoding the S. aureus SrtA variant, and in some embodiments the nucleic acid is set forth in SEQ ID NO: 4.

SEQ ID NO: 4
AAACCACATATCGATAATTATCTTCACGATAAAGATAAAGATGAAAAGAT
TGAACAATATGATAAAAATGTAAAAGAACAGGCGAGTAAAGATAAAAAGC
AGCAAGCTAAACCTCAAATTCCGAAAGATAAATCGAAAGTGGCAGGCTAT
ATTGAAATTCCAGATGCTGATATTAAAGAACCAGTATATCCAGGACCAGC
AACACGTGAACAATTAAATAGAGGTGTAAGCTTTGCAGAAGAAAATGAAT
CACTAGATGATCAAAATATTTCAATTGCAGGACACACTTTCATTGGCCGT
CCGAACTATCAATTTACAAATCTTAAAGCAGCCAAAAAAGGTAGTATGGT
GTACTTTAAAGTTGGTAATGAAACACGTAAGTATAAAATGACAAGTATAA
GAAATGTTAAGCCTACAGCTGTAGGAGTTCTAGATGAACAAAAAGGTAAA
GATAAACAATTAACATTAATTACTTGTGATGATCTTAATCGGGAGACAGG
CGTTTGGGAAACACGTAAAATCTTGGTAGCTACAGAAGTCAAA

In some embodiments, a sortase A variant may comprise any one or more of the following: an S residue at position 94 (S94) or an R residue at position 94 (R94), a K residue at position 105 (K105), an A residue at position 108 (A108) or a Q residue at position 108 (Q 108), a G residue at position 124 (G124), an N residue at position 160 (N160), an A residue at position 165 (A165), a R residue at position 189 (R189), an E residue at position 190 (E190), a T residue at position 196 (T196), and an L residue at position 200 (L200) (numbered according to the numbering of a wild type SrtA, e.g., SEQ ID NO: 1), optionally with about 25-60 (e.g., 25, 30, 35, 40, 45, 50, 55, 56, 57, 58, 59, or 60) amino acids being removed from N-terminus of the wild type S. aureus SrtA. For example, in some embodiments a sortase A variant comprises two, three, four, or five of the afore-mentioned mutations relative to a wild type S. aureus SrtA (e.g., SEQ ID NO: 1). In some embodiments a sortase A variant comprises an S residue at position 94 (S94) or an R residue at position 94 (R94), and also an N residue at position 160 (N160), an A residue at position 165 (A 165), and a T residue at position 196 (T196) relative to a wild type S. aureus SrtA (e.g., SEQ ID NO: 1). For example, in some embodiments, a sortase A variant comprises P94S or P94R, and also D160N, D165A, and K196T relative to a wild type S. aureus SrtA (e.g., SEQ ID NO: 1). In some embodiments a sortase A variant comprises an S residue at position 94 (S94) or an R residue at position 94 (R94) and also an N residue at position 160 (N160), A residue at position 165 (A165), an E residue at position 190, and a T residue at position 196 relative to a wild type S. aureus SrtA (e.g., SEQ ID NO: 1). For example, in some embodiments a sortase A variant comprises P94S or P94R, and also D160N, D165A, K190E, and K196T relative to a wild type S. aureus SrtA (e.g., SEQ ID NO: 1). In some embodiments a sortase A variant comprises an R residue at position 94 (R94), an N residue at position 160 (N160), a A residue at position 165 (A165), E residue at position 190, and a T residue at position 196 relative to a wild type S. aureus SrtA (e.g., SEQ ID NO: 1). In some embodiments a sortase comprises P94R, D160N, D165A, K190E, and K196T relative to a wild type S. aureus SrtA (e.g., SEQ ID NO: 1). In some embodiments, the S. aureus SrtA variants may have 25-60 (e.g., 25, 30, 35, 40, 45, 50, 55, 56, 57, 58, 59 or 60) amino acids being removed from N-terminus.

In some embodiments, a sortase A variety having higher transamidase activity than a naturally occurring sortase A may be used. In some embodiments the activity of the sortase A variety is at least about 10, 15, 20, 40, 60, 80, 100, 120, 140, 160, 180, or 200 times as high as that of wild type S. aureus sortase A. In some embodiments such a sortase variant is used in a composition or method of the present disclosure. In some embodiments a sortase variant comprises any one or more of the following substitutions relative to a wild type S. aureus SrtA: P94S/R, E105K, E108A, E108Q, D124G, D160N, D165A, Y187L, E189R, K190E, K196T and F200L mutations. In some embodiments, the SrtA variant may have 25-60 (e.g., 30, 35, 40, 45, 50, 55, 59 or 60) amino acids being removed from N-terminus.

In some embodiments, the amino acid mutation positions are determined by an alignment of a parent S. aureus SrtA (from which the S. aureus SrtA variant as described herein is derived) with the polypeptide of SEQ ID NO: 1, i.e., the polypeptide of SEQ ID NO: 1 is used to determine the corresponding amino acid sequence in the parent S. aureus SrtA. Methods for determining an amino acid position corresponding to a mutation position as described herein is well known in the art. Identification of the corresponding amino acid residue in another polypeptide can be confirmed by using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. Based on above well-known computer programs, it is routine work for those of skills to determine the amino acid position of a polypeptide of interest as described herein.

In some embodiments, the sortase variant may further comprise 1, 2, 3, 4, 5, 6, 7, 8, 9, 10, 11, 12, 13, 14, or 15 conservative amino acid mutations. Conservative amino acid mutations that will not substantially affect the activity of a protein are well known in the art.

In some embodiments, the present disclosure provides a method of identifying a sortase variant candidate for conjugating an agent to at least one endogenous, non-engineered membrane protein of a red blood cell, comprising contacting the red blood cell with a sortase substrate that comprises a sortase recognition motif and an agent, in the presence of the sortase variant candidate under conditions suitable for the sortase variant candidate to conjugate the sortase substrate to the at least one endogenous, non-engineered membrane protein of the RBC by a sortase-mediated reaction, preferably by a sortase-mediated glycine conjugation and/or a sortase-mediated lysine side chain ε-amino group conjugation. In some embodiments, the sortase-mediated glycine conjugation and/or the sortase-mediated lysine side chain ε-amino group conjugation occur at least on glycine₍ₙ₎ and/or lysine ε-amino group at internal sites of the extracellular domain of the at least one endogenous, non-engineered membrane protein, preferably n being 1 or 2. In some embodiments, the method further comprises selecting the sortase variant capable of conjugating an agent to at least one endogenous, non-engineered membrane protein of a red blood cell.

In some embodiments, the present disclosure contemplates administering a sortase and a sortase substrate to a subject to conjugate in vivo the sortase substrate to red blood cells. For this purpose, it is desirable to use a sortase that has been further modified to enhance its stabilization in circulation and/or reduce its immunogenicity. Methods for stabilizing an enzyme in circulation and for reducing enzyme immunogenicity are well known in the art. For example, in some embodiments, the sortase has been PEGylated and/or linked to an Fc fragment at a position that will not substantially affect the activity of the sortase.

### Irreversible Linkers

Since a SrtA-mediated protein-cell conjugation is a reversible reaction, to improve the efficiency of cell labeling, it would be beneficial to minimize the occurrence of reverse reactions. One solution to increase the product yield is to increase the concentration of the reaction substrates, but it may be difficult to achieve a very high concentration for macromolecular proteins in practical applications; and even if the high concentration could be reached, the high cost may limit the use of this technology. Another solution is to continuously remove the products from the reaction system so that the reaction will not stop due to equilibrium, but since the reaction is carried out on the cell, product separation may be difficult. The inventors of the present application found that surprisingly for cell labelling, the reverse reaction can be prevented by introducing hydroxyacetyl-like byproduct which is not a substrate for the reverse reaction, thus rendering the labeling reaction irreversible.

To obtain hydroxyacetyl-like byproduct, the present disclosure contemplates using a sortase recognition motif comprising an unnatural amino acid, preferably located at position 5 from the direction of N-terminal to C-terminal of the sortase recognition motif. In some embodiments, the unnatural amino acid is a substituted or unsubstituted hydroxyl carboxylic acid having a formulae of CH₂OH—(CH₂)ₙ—COOH, n being an integer from 0 to 5, e.g., 0, 1, 2, 3, 4 and 5, preferably n=0. In some embodiments, the sortase recognition motif comprising an unnatural amino acid may be selected from a group consisting of LPXT*Y, LPXA*Y, LPXS*Y, LPXL*Y, LPXV*Y, LGXT*Y, LAXT*Y, LSXT*Y, NPXT*Y, MPXT*Y, IPXT*Y, SPXT*Y, VPXT*Y and YPXR*Y, wherein * represents the optionally substituted hydroxyl carboxylic acid; and X and Y independently represent any amino acid. In some embodiments, the sortase recognition motif comprising a unnatural amino acid may be selected from a group consisting of LPXT*G, LPXA*G, LPXS*G, LPXL*G, LPXV*G, LGXT*G, LAXT*G, LSXT*G, NPXT*G, MPXT*G, IPXT*G, SPXT*G, VPXT*G, YPXR*G, LPXT*S and LPXT*A, preferably M is LPET*G with * preferably being 2-hydroxyacetic acid. In some embodiments, Leu-Pro-Glu-Thr-2-hydroxyacetic acid-Gly (LPET-(2-hydroxyacetic acid)-G) is used as a linker to ensure that the byproduct would make the reaction irreversible.

To introduce the irreversible linker to an agent, in some embodiments, the sortase recognition motif comprising an unnatural amino acid as a linker is chemically synthesized and can be directly conjugated to an agent such as a protein or polypeptide.

In some embodiments, the sortase recognition motif comprising an unnatural amino acid can be conjugated to an agent by various chemical means to generate a desired sortase substrate. These methods may include chemical conjugation with bifunctional cross-linking agents such as, e.g., an NHS ester-maleimide heterobifunctional crosslinker to connect a primary amine group with a reduced thiol group. Other molecular fusions may be formed between the sortase recognition motif and the agent, for example through a spacer.

Various chemical conjugation means, bifunctional crosslinker or spacer can be used in the present disclosure, including but not limited to: (1) zero-length type (e.g., EDC; EDC plus sulfo NHS; CMC; DCC; DIC; N,N′-carbonyldiimidazolc; Woodward's reagent K); (2) amine-sulfhydryl type such as an NHS ester-maleimide heterobifunctional crosslinker (e.g., Maleimido carbonic acid (C₂-4) (e.g., 6-Maleimidohexanoic acid and 4-Malcimidobutyric acid); EMCS; SPDP, LC-SPDP, sulfo-LC-SPDP; SMPT and sulfo-LC-SMPT; SMCC, LC-SMCC and sulfo-SMCC; MBS and sulfo-MBS; SIAB and sulfo-SIAB; SMPB and sulfo-SMPB; GMBS and sulfo-GMBS; SIAX and SIAXX; SIAC and SIACX; NPIA); (3) homobifunctional NHS esters type (e.g., DSP; DTSSP; DSS; DST and Sulfo-DST; BSOCOES and Sulfo-BSOCOES; EGS and Sulfo-EGS); (4) homobifunctional imidoesters type (e.g., DMA; DMP; DMS; DTBP); (5) carbonyl-sulfydryl type (e.g., KMUH; EMCH; MPBH; M2C2H; PDPH); (6) sulfhydryl reactive type (e.g., DPDPB; BMH; HBVS); (7) sulfhydryl-hydroxy type (e.g., PMPI); or the like.

In some embodiments, an amine-sulfhydryl type or an NHS ester-maleimide heterobifunctional crosslinker is a particularly preferable spacer that can be used herein to conjugate a uric acid degrading peptide to the sortase recognition motif comprising an unnatural amino acid as set forth herein. In certain embodiments, the NHS ester-maleimide heterobifunctional crosslinker such as 6-Maleimidohexanoic acid and 4-Maleimido butyric acid are particularly useful spacers for the construction of desired sortase substrates. The NHS ester-maleimide heterobifunctional crosslinker such as 6-Maleimidohexanoic acid and 4-Maleimido butyric acid can undergo a Michael addition reaction with an exposed sulfhydryl group, e.g., on an exposed cysteine, but this reaction will not occur with an unexposed cysteine. In one embodiment, 6-Maleimidohexanoic acid was introduced in the irreversible linker of the present disclosure, to obtain 6-Maleimidohexanoic acid-Leu-Pro-Glu-Thr-2-hydroxyacetic acid-Gly as shown in FIG. 3. In some embodiments, a cysteine residue is or has been added to the C-terminal of the agent to provide an exposed cysteine.

In some embodiments, the spacers may additionally include a purification tag (for purification after expression) or a linker that is used to maintain the enzyme in a sufficient distance from the sortase binding site, which may be favored in consideration of the steric effect. Exemplary linkers include, but are not limited to, a poly-glycine poly-serine linker (e.g., (GS)₃, GGGGSGGGG, GGGGSGGGGS), and other exemplary linker such as PSTSTST and EIDKPSQ.

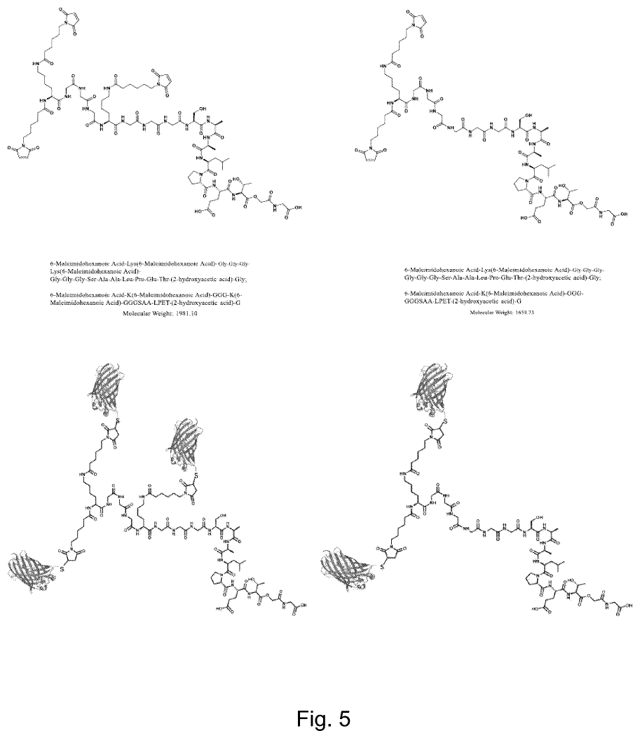

By using the spacers as described herein, especially NHS ester-maleimide heterobifunctional crosslinkers such as 6-Maleimidohexanoic acid and 4-Maleimido butyric acid, the inventors successfully designed linkers with different structures, including double forks, triple forks and multiple forks. These different linkers can be used to label RBCs according to actual needs, for example to obtain multi-modal therapeutics. In the multi-fork structure design of some embodiments, one or more spacers can be linked to the amino group of N-terminal amino acid and/or the amino group of the side chain of lysine and the same or different agents like proteins or polypeptides can be linked to the one or more spacers, as shown in FIG. 5. This technology could further expand the variety of agents like proteins for cell labeling and improve the efficiency of RBC engineering.

The spacers described above can also be used to conjugate the agent of interest to a sortase recognition motif without an unnatural amino acid as described herein above.

### Sortase Substrates

Substrates suitable for a sortase-mediated conjugation can readily be designed. A sortase substrate may comprises a sortase recognition motif and an agent. For example, an agent such as polypeptides can be modified to include a sortase recognition motif at or near their C-terminus, thereby allowing them to serve as substrates for sortase. The sortase recognition motif need not be positioned at the very C-terminus of a substrate but should typically be sufficiently accessible by the enzyme to participate in the sortase reaction. In some embodiments a sortase recognition motif is considered to be “near” a C-terminus if there are no more than 5, 6, 7, 8, 9, 10 amino acids between the most N-terminal amino acid in the sortase recognition motif (e.g., L) and the C-terminal amino acid of the polypeptide. A polypeptide comprising a sortase recognition motif may be modified by incorporating or attaching any of a wide variety of moieties (e.g., peptides, proteins, compounds, nucleic acids, lipids, small molecules and sugars) thereto.

In some embodiments, the present disclosure provides a sortase substrate comprising a structure of (A¹-Sp)ₘ-M, in which A¹ represents an agent, Sp represents the optional spacers, m being an integer greater than or equal to 1, preferably m=1 to 3; and M represents a sortase recognition motif or a sortase recognition motif comprising an unnatural amino acid as set forth herein.

In some embodiments, the Sp is selected from a group consisting of the following types of crosslinkers: (1) zero-length type; (2) amine-sulfhydryl type; (3) homobifunctional NHS esters type; (4) homobifunctional imidoesters type; (5) carbonyl-sulfydryl type; (6) sulfhydryl reactive type; and (7) sulfhydryl-hydroxy type; preferably the one or more Sp is an NHS ester-maleimide heterobifunctional crosslinker such as 6-Maleimidohexanoic acid and 4-Maleimidobutyric acid and the agent comprises an exposed sulfydryl, preferably an exposed cysteine, more preferably a terminal cysteine, most preferably a C-terminal cysteine. In some embodiments, when two or more spacers are presents, the agents linked to the spacers can be the same or different.

### Agents

In some embodiments, the agent comprises a uric acid degrading polypeptide, or a combination of a uric acid degrading polypeptide and a uric acid transporter.

As used herein, the term “a uric acid degrading peptide” refers to any polypeptide or enzyme that is involved in catabolizing or degrading uric acid (UA). Exemplary examples of uric acid degrading polypeptides include, but not limited to, urate oxidase or uric acid oxidase (also known as uricase or UOX), allantoinase and allantoicase. In an embodiment, a uric acid degrading polypeptide has uric acid as its substrate. In an embodiment, a uric acid degrading polypeptide catalyzes the hydrolysis of uric acid.

In one aspect, the present disclosure provides a red blood cell (RBC) having one or more uric acid degrading polypeptide or a variant thereof linked thereto. In some embodiments, the RBC comprises more than one (e.g., two, three, four, five, or more) polypeptides, each comprising at least one uric acid degrading polypeptide or a variant thereof. In some embodiments, the cells described herein comprise more than one type of polypeptide, wherein each polypeptide comprises a uric acid degrading polypeptide, and wherein the uric acid degrading polypeptides are not the same (e.g., the uric acid degrading polypeptides may comprise different types of uric acid degrading polypeptides, or variants of the same type of uric acid degrading polypeptide). For example, in some embodiments, the RBC may comprise a first polypeptide comprising a uricase, or a variant thereof, and a second polypeptide comprising a uric acid degrading polypeptide that is not a uricase.

Many uric acid degrading polypeptides are known in the art and may be used as described herein. For example, the uric acid catabolism pathway includes several uric acid degrading enzymes. Urate oxidase o uricase is the first of three enzymes to convert uric acid to S-(+)-allantoin (allantoin). After uric acid is converted to 5-hydroxyisourate by urate oxidase, 5-hydroxyisourate (HIU) is converted to 2-oxo-4-hydroxy-4-carboxy-5-ureidoimidazoline (OHCU) by HIU hydrolase, and then to S-(+)-allantoin (allantoin) by 2-oxo4-hydroxy4-carboxy-5-ureidoimidazoline decarboxylase (OHCU decarboxylase). Allantoin is converted to allantoate by allantoinase and then to urea by allantoicase. Any one or more of the enzymes involved in uric acid catabolism (i.e., uric acid degrading polypeptides) can be included in the cells described herein.

In some embodiments, the at least one uric acid degrading polypeptide is selected from the group consisting of a uricase, a 5-hydroxyisourate (HIU) hydrolase, an 2-oxo-4-hydroxy-4-carboxy-5-ureidoimidazoline (OHCET), and the variants thereof. In some embodiments, the uric acid degrading polypeptide comprises or consists of a variant of the wild-type uric acid degrading polypeptide having at least at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of a corresponding wild-type uric acid degrading polypeptide

In some embodiments, the at least one uric acid degrading polypeptide or variant thereof can be derived from any source or species, e.g., mammalian, fungal, plant or bacterial sources, or can be obtained by recombinant engineering.

Uricase (also referred to as UO, urate oxidase, urate:oxygen oxidoreductase (E.C. 1.7.3.3)) is an enzyme in the purine degradation pathway that catalyzes the oxidation of uric acid to 5-hydroxyisourate.

In some embodiments, the uricase, or uricase variant, is obtained from a fungal (including yeast and Aspergillus flavus) source. In some embodiments, the uricase is derived from Candida utilis (e.g., as described in U.S. Pat. No. 6,913,915, and contained in pegsiticase (3Sbio/Selecta Biosciences, Inc.)). In some embodiments, the uricase is the Aspergillus flavus uricase contained in rasburicase (ELITEK®; FASTURTEC®, Sanofi Genzyme).

In some embodiments, the uricase or uricase variant is derived from a bacterium, such as bacterium belonging to Anthrobacter (e.g., Anthrobacter globiformis), Streptomyces (e.g., Streptomyces cyanogenus, Streptomyces cellulosae and Streptomyces sulfureus), Bacillus (e.g., Bacillus subtilis, Bacillus megatherium, Bacillus thermocatenulatus, Bacillus fastidiosus, and Bacillus cereus), Pseudomonas aeruginosa, Cellumonas flavigena, or E. coli.

In some embodiments, the uricase or uricase variant is derived from a mammal, for example a pig, bovine, sheep, goat, baboon, rhesus macaque (Macaca mulatto), mouse (e.g., Mus musculus), rabbit, zebra fish (Danio rerio), or domestic animal.

In some embodiments, the uricase comprises an amino acid sequence of SEQ ID NO: 27 as set forth below:

MSAVKAARYGKDNVRVYKVHKDEKTGVQTVYEMTVCVLLEGEIETSYTKA
DNSVIVATDSIKNTIYITAKQNPVTPPELFGSILGTHFIEKYNHIHAAHV
NIVCHRWTRMDIDGKPHPHSFIRDSEEKRNVQVDVVEGKGIDIKSSLSGL
TVLKSTNSQFWGFLRDEYTTLKETWDRILSTDVDATWQWKNFSGLQEVRS
HVPKFDATWATAREVTLKTFAEDNSASVQATMYKMAEQILARQQLIETVE
YSLPNKHYFEIDLSWHKGLQNTGKNAEVFAPQSDPNGLIKCTVGRSSLKS
KLAA

In some embodiments, the uricase comprises a variant of a uricase having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 27. In some embodiments, the uricase variant possesses a function of the uricase from which it was derived (e.g., the ability to catalyze the oxidation of uric acid (urate) to 5-hydroxyisourate).

In some embodiments, the uricase comprises a fragment of a wild-type uricase. In some embodiments, the fragment of the uricase comprises at least 80, at least 90, at least 100, at least 110, at least 120, at least 130, at least 140, at least 150 or at least 160 amino acid residues (e.g., contiguous amino acid residues) of SEQ ID NO: 27 or a variant thereof. In some embodiments, fragments or variants of the uricase retain at least 80%, at least 85%, at least 90%, at least 95%, at least 98% or at least 99% of the function (e.g., the ability to catalyze the oxidation of uric acid (urate) to 5-hydroxyisourate) as compared to the uricase from which it was derived.

As used herein, the term “a uric acid transporter” refers to a polypeptide that is capable of regulating uric acid transport and thereby regulating plasma uric acid levels.

In some embodiments, the agent linked to the RBC provided in the disclosure additionally comprises a uric acid transporter or a variant thereof. In some embodiments, the agent additionally comprises at least one (e.g., one, two, three, four, or more) polypeptides comprising a uric acid transporter.

In another aspect, the disclosure provides a red blood cell (RBC) having linked thereto a combination of a uric acid degrading polypeptide (e.g., uricase) or a variant or fragment thereof and a uric acid transporter or a variant or fragment thereof. Without wishing to be bound by any particular theory, BRCs having linked thereto both a uric acid degrading polypeptide and a uric acid transporter can improve turnover of uric acid (e.g., the catalysis of uric acid) by facilitating the transfer of uric acid from the uric acid transporter to the uric acid degrading polypeptide.

In some embodiments, the uric acid transporter is selected from the group consisting of URAT1 (also referred to as uric acid transporter 1; SLC22A12; solute carrier family 22 member 12), GLUT9 (also referred to as SLC2A9; Solute Carrier Family 2 Member 9), OAT4 (also referred to as organic anion transporter 4; SLC22A9; Solute Carrier Family 22 Member 11), OAT1 (also referred to as organic anion transporter 1; SLC22A6; Solute Carrier Family 22 Member 6), OAT3 (also referred to as organic anion transporter 3; SLC22A8; Solute Carrier Family 22 Member 8), Gal-9 (also referred to as galectin-9; UAT; Lectin, Galactoside-Binding, Soluble, 9), ABCG2 (also referred to as ATP-binding cassette sub-family G member 2), SLC34A2 (also referred to as sodium-dependent phosphate transport protein 1; Solute Carrier Family 34 Member 2), MRP4 (also referred to as multidrug resistance-associated protein 4; ABCC4), OAT2, NPT1 (also referred to Na(+)/PI cotransporter 1, Solute Carrier Family 17 Member 1, SLC17A1, and NAPI-1), NPT4 (also referred to as Na(+)/PI cotransporter 4, Solute Carrier Family 17 Member 3, SLC17A3, and GOUT4), and MCT9 (also referred to as monocarboxylate transporter 9, Solute Carrier Family 16 Member 9, SLC16A9). In some embodiments, the uric acid transporter is a human uric acid transporter.

In some embodiments, the uric acid transporter comprises a URAT1 comprising the amino acid sequence set forth in SEQ ID NO: 28 below:

MASDVGGGRVTMAMVSMWCTSMNSAAVSHRCWADNSTAASGSSAASGNRH
CRRRWDNATATSWSADTCVDGWVYDRSTSTVAKWNVCDSHAKMASYAGVG
AAACGASDRWASARWTTGRDWGWRVAANGKGAVDTTVSAMRSMGASGTRM
GRRTCSTCWAGTGADAGSNMGVVDAKMGASHGRRTAASAGCANTVHMGAR
SAAVGGGVGAATYTYSSTVRMTAVGGMAARGGAGVRGVHGWVYGTVVSGA
ATSDTDVNAVKKATHGTGNSVKST

In some embodiments, the uric acid transporter comprises a variant of a URAT1 having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 28. In some embodiment the variant of the uric acid transporter possesses a function of the wild-type uric acid transporter from which it was derived (e.g., the ability to import uric acid).

In some embodiments, the uric acid transporter comprises a fragment of a URAT1, a GLUT9, a OAT4, a OAT1, a OAT3, a Gal-9, an ABCG2, a SLC34A2, a MRP4, an OAT2, a NPT1, a NPT4, or a MCT9. In some embodiments, the fragment of the uric acid transporter comprises at least 80, at least 90, at least 100, at least 110, at least 120, at least 130, at least 140, at least 150 or at least 160 amino acid residues (e.g., contiguous amino acid residues) of SEQ ID NO: 28 or a variant thereof. In some embodiments, fragments or variants of the uric acid transporter retain at least 80%, at least 85%, at least 90%, at least 95%, at least 98% or at least 99% of the function (e.g., the ability to import uric acid) as compared to the uric acid transporter from which they were derived.

### Methods for Covalently Modifying Endogenous, Non-Engineered Membrane Proteins of RBCs

In an aspect, the present disclosure provides a method for covalently modifying at least one endogenous, non-engineered membrane protein of a red blood cell, comprising contacting the RBC with a sortase substrate that comprises a sortase recognition motif and an agent as described herein, in the presence of a sortase under conditions suitable for the sortase to conjugate the sortase substrate to the at least one endogenous, non-engineered membrane protein of the RBC by a sortase-mediated reaction, preferably by a sortase-mediated glycine conjugation and/or a sortase-mediated lysine side chain conjugation. In some embodiments, the sortase-mediated glycine conjugation and/or the sortase-mediated lysine side chain ε-amino group conjugation occur at least on glycine₍ₙ₎ and/or lysine ε-amino group in the extracellular domain (for example at internal sites of the extracellular domain) of the at least one endogenous, non-engineered membrane protein, preferably n being 1 or 2. In some embodiments, without being limited to the theory, the sortase-mediated glycine conjugation may also occur at exposed glycine_{(n=1 or 2)} of previously unreported membrane proteins due to tissue-specific mRNA splicing and protein translation during erythropoiesis. In some embodiments, the sortase-mediated lysine side chain ε-amino group conjugation occur at C-amino group of terminal lysine or internal lysine of the extracellular domain.

It would be understood that those of ordinary skills are able to select conditions (e.g., optimal temperature, pH) suitable for the sortase to conjugate the sortase substrate to the at least one endogenous, non-engineered membrane protein according to the nature of sortase substrate, the type of sortase and the like.

### Uses

In some aspects, the present disclosure provides a method for treating or preventing a disorder, condition or disease associated with an elevated uric acid level in a subject in need thereof, comprising administering the red blood cell or composition as described herein to the subject.

As used herein, the term an “elevated uric acid level” refers to any level of uric acid in a subject's serum that may lead to an undesirable result or would be deemed by a clinician to be elevated. In an embodiment, an elevated uric acid level refers to a level of uric acid considered to be above normal by a clinician. In an embodiment, the subject can have a serum uric acid level of >5 mg/dL, >6 mg/dL, >7 mg/dL or 8 mg/dL.

A disorder, condition or disease associated with an elevated uric acid level can include hyperuricemia, gout (e.g., chronic refractory gout, gout tophus and gouty arthritis), metabolic syndrome, tumor lysis syndrome, Lesch-Nyhan syndrome, cardiovascular disease, diabetes, hypertension, renal disease, or uric acid nephrolithiasis. Such disorders can be treated with the red blood cells having linked thereto a uric acid degrading polypeptide, or a combination of a uric acid degrading polypeptide and a uric acid transporter polypeptide.

As used herein, the term “hyperuricemia” refers to a disease or disorder typically associated with elevated levels of uric acid. As used herein, the term “gout” generally refers to a disorder or condition associated with the buildup of uric acid, such as deposition of uric crystals in tissues and joints, and/or a clinically relevant elevated serum uric acid level.

In some embodiments, the present disclosure provides a method for reducing an elevated uric acid level in a subject in need thereof, comprising administering the red blood cell or composition as described herein to the subject. In some embodiments, the uric acid level in the subject receiving the treatment decreases by at least 10%, at least 20%, at least 30%, at least 40%, at least 50%, at least 60%, at least 70%, at least 80%, or decreases to a normal level.

As used herein, “treating”, “treat” or “treatment” refers to a therapeutic intervention that at least partly ameliorates, eliminates or reduces a symptom or pathological sign of a pathogen-associated disease, disorder or condition after it has begun to develop. Treatment need not be absolute to be beneficial to the subject. The beneficial effect can be determined using any methods or standards known to the ordinarily skilled artisan.

As used herein, “preventing”, “prevent” or “prevention” refers to a course of action initiated prior to infection by, or exposure to, a pathogen or molecular components thereof and/or before the onset of a symptom or pathological sign of the disease, disorder or condition, so as to prevent infection and/or reduce the symptom or pathological sign. It is to be understood that such preventing need not be absolute to be beneficial to a subject. A “prophylactic” treatment is a treatment administered to a subject who does not exhibit signs of the disease, disorder or condition, or exhibits only early signs for the purpose of decreasing the risk of developing a symptom or pathological sign of the disease, disorder or condition.

In some embodiments, the method as described herein further comprises administering the conjugated red blood cells to a subject, e.g., directly into the circulatory system, e.g., intravenously, by injection or infusion.

In some embodiments, a subject receives a single dose of cells, or receives multiple doses of cells, e.g., between 2 and 5, 10, 20, or more doses, over a course of treatment. In some embodiments a dose or total cell number may be expressed as cells/kg. For example, a dose may be about 10³, 10⁴, 10⁵, 10⁶, 10⁷, 10⁸ cells/kg. In some embodiments a course of treatment lasts for about 1 week to 12 months or more e.g., 1, 2, 3 or 4 weeks or 2, 3, 4, 5 or 6 months. In some embodiments a subject may be treated about every 2-4 weeks. One of ordinary skills in the art will appreciate that the number of cells, doses, and/or dosing interval may be selected based on various factors such as the weight, and/or blood volume of the subject, the condition being treated, response of the subject, etc. The exact number of cells required may vary from subject to subject, depending on factors such as the species, age, weight, sex, and general condition of the subject, the severity of the disease or disorder, the particular cell(s), the identity and activity of agent(s) conjugated to the cells, mode of administration, concurrent therapies, and the like.

### Composition

In another aspect, the present disclosure provides a composition comprising the red blood cell as described herein and optionally a physiologically acceptable carrier, such as in the form of a pharmaceutical composition, a delivery composition or a diagnostic composition or a kit.

In some embodiments, the composition may comprise a plurality of red blood cells. In some embodiments, at least a selected percentage of the cells in the composition are modified, i.e., having an agent conjugated thereto by sortase. For example, in some embodiments at least 5%, 10%, 15%, 20%, 25%, 30%, 40%, 50%. 60%, 70%, 80%. 90%, 95%, 96%, 97%, 98%, 99%, or more of the cells have an agent conjugated thereto. In some embodiments, two or more red blood cells or red blood cell populations conjugated with different agents are included.

In some embodiments, a composition comprises sortagged blood red cells, wherein the cells are sortagged with any agent of interest. In some embodiments, a composition comprises an effective amount of cells, e.g., up to about 10⁴ cells, e.g., about 10, 10², 10³, 10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, 10¹², 5×10¹², 10¹³, 5×10¹³, or 10¹⁴ cells. In some embodiments the number of cells may range between any two of the afore-mentioned numbers.

As used herein, the term “an effective amount” refers to an amount sufficient to achieve a biological response or effect of interest, e.g., reducing one or more symptoms or manifestations of a disease or condition or modulating an immune response. In some embodiments a composition administered to a subject comprises up to about 10¹⁴ cells, e.g., about 10³, 10⁴, 10⁵, 10⁶, 10⁷, 10⁸, 10⁹, 10¹⁰, 10¹¹, 10¹², 10¹³ or 10¹⁴ cells, or any intervening number or range.

In another aspect, the composition of the present aspect may comprise a sortase and a sortase substrate but without red blood cells. The composition will be administered to the circulatory system in a subject and upon contacting red blood cells in vivo, the sortase conjugates the sortase substrate to at least one endogenous, non-engineered membrane protein of the red blood cells by a sortase-mediated reaction as described herein. In this form of composition, there will be no risk of incompatibility of red blood cells as well as other risks, such as bacterial or viruses contamination from donor cells. In some embodiments, the sortase has been further modified to enhance its stabilization in circulation by e.g., PEGylation or Fusion to Fc fragment and/or reduce its immunogenicity.

As used herein, the term “a physiologically acceptable carrier” is meant a solid or liquid filler, diluent or encapsulating substance that may be safely used in systemic administration. Depending upon the particular route of administration, a variety of carriers, diluent and excipients well known in the art may be used. These may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates, water and pyrogen-free water.

It will be appreciated by those skilled in the art that other variations of the embodiments described herein may also be practiced without departing from the scope of the invention. Other modifications are therefore possible.

Although the disclosure has been described and illustrated in exemplary forms with a certain degree of particularity, it is noted that the description and illustrations have been made by way of example only. Numerous changes in the details of construction and combination and arrangement of parts and steps may be made. Accordingly, such changes are intended to be included in the invention, the scope of which is defined by the claims.

### Examples

### Example 1. Mg SrtA-Mediated Protein-Cell Conjugation

### Methods

Recombinant Protein Expression and Purification in E. coli

Mg SrtA (SEQ ID NO: 3/4), wt SrtA (SEQ ID NO: 1 with 25 amino acids removed from N-terminus) and eGFP-LPETG cDNA(SEQ ID NO: 35/36) were cloned in pET vectors and transformed in E. coli BL21(DE3) cells for protein expression. Transformed cells were cultured at 37° C. until the OD₆₀₀ reaching 0.6-0.8 and then 500 μM IPTG were added for 4 hrs at 37° C. After that, cells were harvested by centrifugation and subjected to lysis by precooled lysis buffer (20 mM Tris-HCl, pH 7.8, 100 mM NaCl). The lysates were proceeded for sonication on ice (5s on, 5s off, 60 cycles, 25% power, Branson Sonifier 550 Ultrasonic Cell Disrupter). All supernatants were filtered by 0.22 μM filter after centrifugation at 14,000 g for 40 min at 4° C. Filtered supernatants were loaded onto HisTrap FF 1 mL column (GE Healthcare) connected to the AKTA design chromatography systems. The proteins were eluted with the elution buffer containing 20 mM Tris-HCl, pH 7.8, 100 mM NaCl and 300 mM imidazole. All eluted fractions were analyzed on a 12% SDS-PAGE gel.

The amino acid sequence of eGFP-LEPTG is as shown in SEQ ID NO: 35 below:

MHHHHHHMVSKGEELFTGVVPILVELDGDVNGHKFSVSGEGEGDATYGKL
TLKFICTTGKLPVPWPTLVTTLTYGVQCFSRYPDHMKQHDFFKSAMPEGY
VQERTIFFKDDGNYKTRAEVKFEGDTLVNRIELKGIDFKEDGNILGHKLE
YNYNSHNVYIMADKQKNGIKVNFKIRHNIEDGSVQLADHYQQNTPIGDGP
VLLPDNHYLSTQSALSKDPNEKRDHMVLLEFVTAAGITLGMDELYKAALP
ETGG*

The nucleotide sequence of eGFP-LEPTG is as shown in SEQ ID NO: 36 below:

ATGCACCACCACCACCACCACATGGTTAGTAAAGGGGAAGAATTATTTAC
CGGCGTGGTGCCGATTCTGGTTGAACTGGACGGCGACGTGAACGGCCACA
AATTCAGCGTTAGCGGCGAGGGCGAAGGTGACGCGACCTACGGCAAGCTG
ACCCTGAAATTTATCTGCACCACCGGCAAGCTGCCGGTGCCGTGGCCGAC
CCTGGTTACCACCCTGACCTACGGTGTTCAGTGCTTCAGCCGTTATCCGG
ACCACATGAAGCAACACGATTTCTTTAAAAGCGCGATGCCGGAGGGCTAC
GTGCAGGAACGTACCATCTTCTTTAAGGACGATGGTAACTATAAAACCCG
TGCGGAAGTGAAGTTCGAAGGCGACACCCTGGTTAACCGTATCGAGCTGA
AGGGTATTGACTTTAAAGAAGATGGCAACATTCTGGGTCACAAACTGGAG
TACAACTATAACAGCCACAACGTGTATATCATGGCGGATAAGCAGAAAAA
CGGCATTAAGGTTAACTTCAAAATCCGTCACAACATTGAAGACGGTAGCG
TGCAACTGGCGGATCACTACCAGCAAAACACCCCGATTGGCGACGGTCCG
GTTCTGCTGCCGGATAACCACTATCTGAGCACCCAAAGCGCGCTGAGCAA
GGACCCGAACGAGAAACGTGATCACATGGTGCTGCTGGAATTTGTTACCG
CGGCGGGTATCACCCTGGGTATGGACGAACTGTATAAGGCGGCGCTGCCG
GAGACCGGCGGTTAA

### Wt SrtA or Mg SrtA-Mediated Enzymatic Labeling of Membrane Proteins

Reactions were performed in a total volume of 200 μL at 37° C. for 2 hrs in PBS buffer while being rotated at a speed of 10 rpm. The concentration of wt SrtA or mg SrtA was 20-40 μM and the biotin-LPETG or GFP-LPETG substrates were at the range of 200-1000 μM. Human or mouse RBCs were washed twice with PBS before enzymatic reactions. The concentration of RBCs in the reaction was from 1×10⁶/mL to 1×10¹⁰/mL. After the reaction, RBCs were washed three times and incubated with Streptavidin-phycoerythrin (PE) at room temperature for 10 min before analyzed by Beckman Coulter CytoFLEX LX or Merck Amnis Image Stream MarkII.

### Enrichment of RBC Membrane Proteins

The biotin-labeled RBCs were resuspended in PBS and sonicated (10s on, 10s off, 3 cycles, 25% power, SONICS VCX150) on ice. Intact cells were removed by centrifugation at 4° C., 300×g for 15 min. Dried powder was obtained by freezing and lyophilizing then incubation with 50 mL of ice-cold 0.1 M sodium carbonate (pH=11) at 4° C. for 1 h with gentle rotation at a speed of 10 rpm. Membranous fractions were pelleted down by ultracentrifugation at 125,000×g at 4° C. for 1 h and then washed twice with Milli-Q water at the same speed for 30 mins. Then the samples were incubated with 2 mL of ice-cold 80% acetone for protein precipitation at −20° C. for 2 hrs. Membrane proteins were collected by centrifugation at 130,000×g at 4° C. for 15 mins. Membrane proteins samples were redissolved in 1% SDS and analyzed by gel electrophoresis using 12% SDS-PAGE.

### In-Gel Digestion

The whole gel was stained by Coomassie blue (H₂O, 0.1% w/v Coomassie brilliant blue R250, 40% v/v methanol and 10% v/v acetic acid) at room temperature with gently shaking overnight then destained with the destaining solution (40% v/v methanol and 10% v/v acetic acid in water). The gel was rehydrated three times in distilled water at room temperature for 10 min with gentle agitation. The protein bands were cut out and further cut off into ca 1×1 mm² pieces, followed by reduction with 10 mM TCEP in 25 mM NH4HCO₃ at 25° C. for 30 min, alkylation with 55 mM IAA in 25 mM NH₄HCO₃ solution at 25° C. in the dark for 30 min, and sequential digestion with rPNGase F at a concentration of 100 unit/ml at 37° C. for 4 hrs, and then digestion with trypsin at a concentration of 12.5 ng/mL at 37° C. overnight (1st digestion for 4 hrs and 2nd digestion for 12 hrs). Tryptic peptides were then extracted out from gel pieces by using 50% ACN/2.5% FA for three times and the peptide solution was dried under vacuum. Dry peptides were purified by Pierce C18 Spin Tips (Thermo Fisher, USA).

### Mass Spectrometry Analysis

Biognosys-11 iRT peptides (Biognosys, Schlieren, CH) were spiked into peptide samples at the final concentration of 10% prior to MS injection for RT calibration. Peptides were separated by Ultimate 3000 nanoLC-MS/MS system (Dionex LC-Packings, Thermo Fisher Scientific™, San Jose, USA) equipped with a 15 cm×75 μm ID fused silica column packed with 1.9 μm 120 Å C18. After injection, 500 ng peptides were trapped at 6 μL/min on a 20 mm×75 μm ID trap column packed with 3 μm 100 Å C18 aqua in 0.1% formic acid, 2% ACN. Peptides were separated along a 60 min 3-28% linear LC gradient (buffer A: 2% ACN, 0.1% formic acid (Fisher Scientific); buffer B: 98% ACN, 0.1% formic acid) at the flowrate of 300 nL/min (108 min inject-to-inject in total). Eluting peptides were ionized at a potential of +1.8 kV into a Q-Exactive HF mass spectrometer (Thermo Fisher Scientific™, San Jose, USA). Intact masses were measured at resolution 60,000 (at m/z 200) in the Orbitrap using an AGC target value of 3E6 charges and a maximum ion injection time of 80 ms. The top 20 peptide signals (charge-states higher than 2+ and lower than +6) were submitted to MS/MS in the HCD cell (1.6 amu isolation width, 27% normalized collision energy). MS/MS spectra were acquired at resolution 30,000 (at m/z 200) in the Orbitrap using an AGC target value of 1E5 charges, a maximum ion injection time of 100 ms. Dynamic exclusion was applied with a repeat count of 1 and an exclusion time of 30 s. The Maxquant (version 1.6.2.6) was used as a search engine with the fixed modification was cysteine (Cys) carbamidomethyl. and methionine (Met) oxidation as a variable modification. Variable modifications contained oxidation (M), deamidation (NQ), GX808-G-N, GX808-G-anywhere, GX808-K-sidechain. (for details, see Table 1). Other parameters were performed as default. Data was searched against the Swissprot Mouse database September 2018) and further filtered the data with FDR≤1%.

### Results

We first characterized the efficacy of mg SrtA-mediated labeling on RBC membranes. Wt SrtA was employed as the control for its recognition of three glycines at the N-terminus of proteins or peptides. Our results showed that >99% of natural mouse or human RBCs were biotin-labeled by mg SrtA in vitro. In contrast, no significant biotin signal was detected on the surface of mouse or human RBCs by wt SrtA nor the mock control group without enzyme (FIGS. 1A and 1B). Western-blot analysis also supported our flow cytometry results demonstrating mg SrtA-mediated biotin labeling of mouse RBCs (FIG. 1C). To further validate this finding, membrane proteins of natural mouse RBCs from the mg SrtA-labeled group or the mock control group were enriched by ultracentrifugation as described[6](FIG. 1D). As expected, significant increases in biotin signals were detected in the mg SrtA-labeled group after the enrichment of RBC membrane proteins [6] (FIG. 1E). To assess the life-span of these surface modified RBCs in vivo, we next transfused biotin-LPETG tagged mouse RBCs, which were simultaneously labeled with a fluorescent dye DiR (1,1′-dioctadecyl-3,3,3′,3′-tetramethylindotricarbocyanine iodide), into wildtype recipient mice. The percentage of DiR and biotin positive RBCs in vivo was analyzed periodically. We found that biotin labeled RBCs by mg SrtA not only showed the same lifespan as the control groups but also remained 90% biotin positive during circulation (FIGS. 1F, 1G and 1H). Imaging analysis also showed convincing biotin signals on the cell surface and normal morphology of mg sortase-labeled RBCs (FIG. 1I). We also sortagged RBCs with eGFP-LPETG and transfused them into wildtype mice. As expected, RBCs conjugated with eGFP by mg SrtA but not by wt SrtA were detected in vivo, and the detected RBCs exhibited normal cellular morphology (FIGS. 1J and 1K). Taken together, our data suggests efficient labeling of peptides and proteins on the surface of natural RBCs mediated by mg SrtA both in vitro and in vivo.

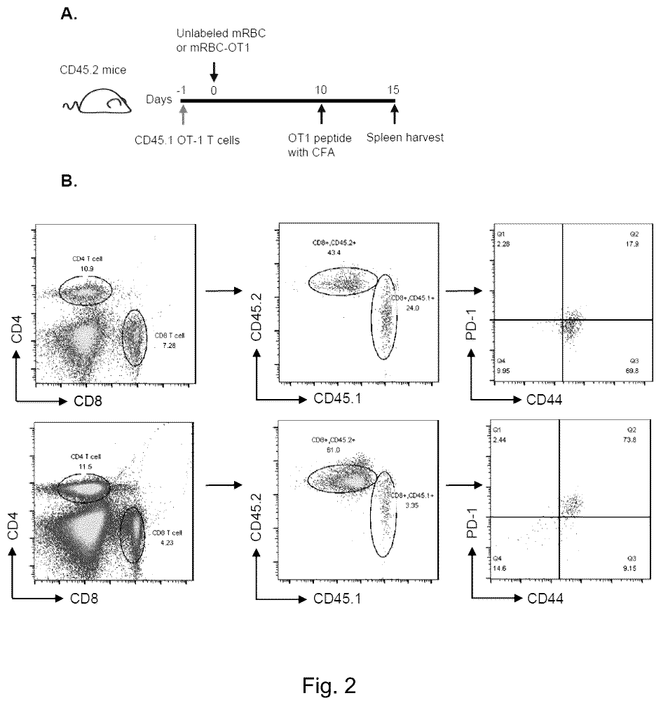

Previous studies have shown that specific-antigen bound RBCs are capable of inducing immunotolerance in several animal disease models[8]. In vitro generated mouse RBCs labeled with OT-1 peptide, which is an ovalbumin (OVA) epitope with SIINFEKL sequence, induce immunotolerances in CD8+ T cells with transgenic TCR recognizing H-2K^{b}-SIINFEKL in an autoimmune disease mouse model[8]. We adoptively transferred CD8⁺ CD45.1 T cells purified from OT1 TCR mice into CD45.2 recipient mice (FIG. 2A). After 24 hrs, same numbers of natural mouse RBCs modified with or without the OT-1 peptide by mg SrtA were injected into the recipient mice. The number of CD8+CD45.1 T cells in the recipient mice receiving OT-1-RBC were ˜7 fold less compared to that in the mice injected with unmodified RBCs after the challenge with OT-1 peptides. Notably, the percentage of PD1′ CD8˜ CD45.1+ T cells are over 4 times more in the mice receiving OT-1-RBC compared to that of recipient mice injected with natural RBCs. There is no change in the expression level of CD44 on the T cells in both groups which is consistent with previous studies[8][9]. These data suggested mg SrtA-modified RBCs carrying OT-1 peptide might induce OT-1 TCR T cell exhaustion but are more convenient and efficient for applications than previous strategies[8].

We next aim to identify the RBC membrane proteins serving as substrates for mg sortase mediated reaction. Biotin labeled RBCs by mg SrtA were analyzed by mass spectrometry (MS); a list of 122 candidate proteins potentially modified with biotin molecules on glycine (G) or the side chain of lysine (K) was detected (Table 1). 68 and 54 of these proteins were modified at glycine and the side chain of lysine, respectively (Tables 2 and 3). 18 of the identified proteins were detected with both modifications (Table 4). Among the total identified proteins, 22 proteins as shown in Table 5 were annotated as membrane proteins. For instance, the calcium-sensing receptor (CaSR), is a G-protein coupled receptor sensing calcium concentration in the circulation. Previous study has identified the presence of CaSR as a membrane protein on the RBC surface, which regulates the erythrocyte homeostasis[10]. Interestingly, biotin signals were detected at the G526 and K527 positions, neither of which is close to the N-terminus of CaSR. In addition, none of the rest 21 membrane proteins have biotin-modified glycine at the N-terminus, either. Therefore, we have identified membrane proteins including CaSR on RBC surface which might be covalently linked to biotin molecules.

Identification of biotin-labeled membrane proteins on RBCs was shown in Table 1. Biotin-labeled or natural RBC membrane proteins enriched from FIG. 1E were subjected to MS analysis. Enriched RBC membrane proteins were loaded into ID gel electrophoresis for last in-gel digestion before being injected into MS instruments. The configuration on MaxQuant software were shown, which is the molecular weight (808 g/mol) increasing on the N-terminal and anywhere glycine and lysine, and the peptide searching was based on the UniProt protein database.

TABLE 1
Variable modifications searched by Maxquant (version 1.6.2.6)

| | | | | New | Specific- |
|---|---|---|---|---|---|
| Name | Composition | Position | Type | teminus | ities |
| GX808-G- | C₃₆H₅₆O₁₁N₈S | Any | Standard | None | G |
| N | | N-term | | | |
| GX808-G- | C₃₆H₅₆O₁₁N₈S | Anywhere | Standard | None | G |
| anywhere | | | | | |
| GX808-K- | C₃₆H₅₆O₁₁N₈S | Anywhere | Standard | None | K |
| side chain | | | | | |

TABLE 2
A list of 68 protein candidates from RBCs modified with biotin-peptide
on glycine(s).

| | | UniProt | Isoform | | | |
|---|---|---|---|---|---|---|
| No. | Protein names | ID | ID | Sequence | Length | Modifications |
| 1 | Extracellular calcium- | CASR | Q9QY96 | LFINEG | 1079 | G-anywhere |
| | sensing receptor | | | K | | |
| | (CaSR) (Parathyroid | | | | | |
| | cell calcium-sensing | | | | | |
| | receptor)(PCaR1) | | | | | |
| 2 | Ryanodine receptor 3 | RYR3 | A2AGL3 | NYMMS | 4863 | G-anywhere |
| | (RYR-3) (RyR3) | | | NGYK | | |
| | (Brain ryanodine | | | | | |
| | receptor-calcium | | | | | |
| | release channel) | | | | | |
| | (Brain-type ryanodine | | | | | |
| | receptor) (Type 3 | | | | | |
| | ryanodine receptor) | | | | | |
| 3 | Rap1 GTPase- | RPGP1 | A2ALS5 | SSAIGIE | 663 | G-anywhere |
| | activating protein 1 | | | NIQEVQ | | |
| | (Rap1GAP) | | | EK | | |
| | (Rap1GAP1) (ARPP- | | | | | |
| | 90) | | | | | |
| 4 | Titin (EC 2.7.11.1) | TITIN | A2ASS6 | DGQVIS | 35213 | G-anywhere |
| | (Connectin) | | | TSTLPG | | |
| | | | | VQISFS | | |
| | | | | DGRAR | | |
| 5 | Inter alpha-trypsin | ITIH4 | A6X935 | GSRSQI | 942 | G-anywhere |
| | inhibitor, heavy chain | | | PR | | |
| | 4 (ITI heavy chain H4) | | | | | |
| | (ITI-HC4) (Inter- | | | | | |
| | alpha-inhibitor heavy | | | | | |
| | chain 4) | | | | | |
| 6 | Trafficking protein | TPC11 | B2RXC1 | VSLAGS | 1133 | G-anywhere |
| | particle complex | | | NVFQIG | | |
| | subunit 11 | | | VQDFVP | | |
| | | | | FVQCK | | |
| 7 | Desmoplakin (DP) | DESP | E9Q557 | NSQGSE | 2883 | G-anywhere |
| | | | | MFGDD | | |
| | | | | DKRR | | |
| 8 | Tumor protein D53 | TPD53 | O54818 | LGMNL | 204 | G-anywhere |
| | (mD53) (Tumor | | | MNELK | | |
| | protein D52-like 1) | | | | | |
| 9 | Inactive serine protease | PRS39 | O70169 | IYGGQI | 367 | G-anywhere |
| | 39 (Inactive testicular | | | AK | | |
| | serine protease 1) | | | | | |
| 10 | Lysine-specific | KDM6A | O70546 | QTLAN | 1401 | G-anywhere |
| | demethylase 6A (EC | | | GPFSAG | | |
| | 1.14.11 .-) (Histone | | | HVPCST | | |
| | demethylase UTX) | | | SR | | |
| | (Ubiquitously | | | | | |
| | transcribed TPR | | | | | |
| | protein on the X | | | | | |
| | chromosome) | | | | | |
| | (Ubiquitously | | | | | |
| | transcribed X | | | | | |
| | chromosome | | | | | |
| | tetratricopeptide repeat | | | | | |
| | protein) | | | | | |
| 11 | Histone-lysine N- | SETB1 | O88974 | QGGQL | 1307 | G-anywhere |
| | methyltransferase | | | RTRPN | | |
| | SETDB1 (EC 2.1.1 .-) | | | MGAVR | | |
| | (ERG-associated | | | | | |
| | protein with SET | | | | | |
| | domain) (ESET) (SET | | | | | |
| | domain bifurcated 1) | | | | | |
| 12 | NF-kappa-B inhibitor- | IKBL1 | O88995 | CPSAM | 381 | G-anywhere |
| | like protein 1 (Inhibitor | | | GIK | | |
| | of kappa B-like | | | | | |
| | protein) (I-kappa-B- | | | | | |
| | like protein) | | | | | |
| | (IkappaBL) (Nuclear | | | | | |
| | factor of kappa light | | | | | |
| | polypeptide gene | | | | | |
| | enhancer in B-cells | | | | | |
| | inhibitor-like 1) | | | | | |
| 13 | Vesicle transport | VTIIA | O89116 | ILTGML | 217 | G-anywhere |
| | through interaction | | | RR | | |
| | with t-SNAREs | | | | | |
| | homolog 1A (Vesicle | | | | | |
| | transport v-SNARE | | | | | |
| | protein Vti1-like 2) | | | | | |
| | (Vti1-rp2) | | | | | |
| 14 | Fructose-bisphosphate | ALDOA | P05064 | LQSIGT | 364 | G-anywhere |
| | aldolase A (EC | | | ENTEEN | | |
| | 4.1.2.13) (Aldolase 1) | | | R | | |
| | (Muscle-type aldolase) | | | | | |
| 15 | T-cell surface | CD3G | P11942 | NTWNL | 182 | G-anywhere |
| | glycoprotein CD3 | | | GNNAK | | |
| | gamma chain (T-cell | | | | | |
| | receptor T3 gamma | | | | | |
| | chain) (CD antigen | | | | | |
| | CD3g) | | | | | |
| 16 | Medium-chain specific | ACAD | P45952 | ELNMG | 421 | G-anywhere |
| | acyl-CoA | M | | QR | | |
| | dehydrogenase, | | | | | |
| | mitochondrial | | | | | |
| | (MCAD)(EC 1.3.8.7) | | | | | |
| 17 | Sulfotransferase 1E1 | ST1E1 | P49891 | EGDVE | 295 | G-anywhere |
| | (ST1E1) (EC 2.8.2.4) | | | KCKED | | |
| | (Estrogen | | | AIFNR | | |
| | sulfotransferase, testis | | | | | |
| | isoform) | | | | | |
| | (Sulfotransferase, | | | | | |
| | estrogen-preferring) | | | | | |
| 18 | P2X purinoceptor 1 | P2RX1 | P51576 | NLSPGF | 399 | G-anywhere |
| | (P2X1)(ATP receptor) | | | NFR | | |
| | (Purinergic receptor) | | | | | |
| 19 | Scavenger receptor | C163A | Q2VLH6 | FQGKW | 1121 | G-anywhere |
| | cysteine-rich type 1 | | | GTVCD | | |
| | protein M130 (CD | | | DNFSK | | |
| | antigen CD163) | | | | | |
| | [Cleaved into: Soluble | | | | | |
| | CD163 (sCD163)] | | | | | |
| 20 | RUN and FYVE | RUFY4 | Q3TYX8 | VEGKGS | 563 | G-anywhere |
| | domain-containing | | | LSGTED | | |
| | protein 4 | | | QRTTEG | | |
| | | | | IQK | | |
| 21 | Coiled-coil domain- | CC177 | Q3UHB | QEGQL | 706 | G-anywhere |
| | containing protein 177 | | 8 | QREK | | |
| 22 | Lysine-specific | KDM5A | Q3UXZ9 | TDIGVY | 1690 | G-anywhere |
| | demethylase 5A (EC | | | GSGKN | | |
| | 1.14.11 .-) (Histone | | | R | | |
| | demethylase | | | | | |
| | JARID1A) (Jumonji/ | | | | | |
| | ARID domain- | | | | | |
| | containing protein 1A) | | | | | |
| | (Retinoblastoma- | | | | | |
| | binding protein 2) | | | | | |
| | (RBBP-2) | | | | | |
| 23 | Down syndrome cell | DSCL1 | Q4VA61 | DGQVII | 2053 | G-anywhere |
| | adhesion molecule-like | | | SGSGVT | | |
| | protein 1 homolog | | | IESK | | |
| 24 | C2 domain-containing | C2CD3 | Q52KB6 | GLPQDL | 2323 | G-anywhere |
| | protein 3 (Protein | | | DLMQK | | |
| | hearty) | | | | | |
| 25 | Vacuolar protein | VP13A | Q5H8C4 | GVAAM | 3166 | G-anywhere |
| | sorting-associated | | | TMDED | | |
| | protein 13A (Chorea- | | | YQQK | | |
| | acanthocytosis protein | | | | | |
| | homolog)(Chorein) | | | | | |
| 26 | Protein KIBRA | KIBRA | QSSXA9 | TQKAE | 1104 | G-anywhere |
| | (Kidney and brain | | | GGSRLQ | | |
| | protein) (KIBRA) | | | ALR | | |
| | (WW domain- | | | | | |
| | containing protein 1) | | | | | |
| 27 | DNA polymerase zeta | REV3L | Q61493 | GNASH | 3122 | G-anywhere |
| | catalytic subunit (EC | | | ATGLFK | | |
| | 2.7.7.7) (Protein | | | | | |
| | reversionless 3-like) | | | | | |
| | (REV3-like) (Seizure- | | | | | |
| | related protein 4) | | | | | |
| 28 | Interferon-induced | IFIT3 | Q64345 | MGEEA | 403 | G-anywhere |
| | protein with | | | EGER | | |
| | tetratricopeptide | | | | | |
| | repeats 3 (IFIT-3) | | | | | |
| | (Glucocorticoid- | | | | | |
| | attenuated response | | | | | |
| | gene 49 protein) | | | | | |
| | (GARG-49) (P49) | | | | | |
| | (IRG2) | | | | | |
| 29 | Potassium-transporting | ATP4A | Q64436 | ILSAQG | 1033 | G-anywhere |
| | ATPase alpha chain 1 | | | CK | | |
| | (EC 7.2.2.19) (Gastric | | | | | |
| | H(+)/K(+) ATPase | | | | | |
| | subunit alpha) (Proton | | | | | |
| | pump) | | | | | |
| 30 | E3 ubiquitin-protein | SH3R1 | Q69ZI1 | LLSGAS | 892 | G-anywhere |
| | ligase SH3RF1 (EC | | | TKR | | |
| | 2.3.2.27) (Plenty of | | | | | |
| | SH3s) (Protein POSH) | | | | | |
| | (RING-type E3 | | | | | |
| | ubiquitin transferase | | | | | |
| | SH3RF1) (SH3 | | | | | |
| | domain-containing | | | | | |
| | RING finger protein 1) | | | | | |
| | (SH3 multiple domains | | | | | |
| | protein 2) | | | | | |
| 31 | Tubulin epsilon and | TEDC2 | Q6GQV | VLGTRS | 436 | G-anywhere |
| | delta complex protein | | 0 | TK | | |
| | 2 | | | | | |
| 32 | FERM domain- | FRMD5 | Q6P5H6 | GPQLQQ | 517 | G-anywhere |
| | containing protein 5 | | | QQWK | | |
| 33 | Vacuolar ATPase | VMA21 | Q78T54 | QWREG | 101 | G-anywhere |
| | assembly integral | | | KQD | | |
| | membrane protein | | | | | |
| | Vma21 | | | | | |
| 34 | APC membrane | AMER1 | Q7TS75 | LFGGKK | 1132 | G-anywhere |
| | recruitment protein 1 | | | | | |
| | (Amer1) (Protein | | | | | |
| | FAM123B) | | | | | |
| 35 | Serine/threonine- | MRCK | Q7TT50 | DIKPDN | 1713 | G-anywhere |
| | protein kinase MRCK | B | | VLLDV | | |
| | beta (EC 2.7.11.1) | | | NGHIR | | |
| | (CDC42-binding | | | | | |
| | protein kinase beta) | | | | | |
| | (DMPK-like beta) | | | | | |
| | (Myotonic dystrophy | | | | | |
| | kinase-related CDC42- | | | | | |
| | binding kinase beta) | | | | | |
| | (MRCK beta) | | | | | |
| | (Myotonic dystrophy | | | | | |
| | protein kinase-like | | | | | |
| | beta) | | | | | |
| 36 | Uncharacterized | CJ062 | Q80Y39 | EMQRES | 304 | G-anywhere |
| | protein C10orf62 | | | GK | | |
| | homolog | | | | | |
| 37 | Dual specificity | DYRK4 | Q8BI55 | NINNNR | 632 | G-anywhere |
| | tyrosine- | | | GGKR | | |
| | phosphorylation- | | | | | |
| | regulated kinase 4 (EC | | | | | |
| | 2.7.12.1) | | | | | |
| 38 | Engulfment and cell | ELMO1 | Q8BPU7 | GALKQ | 727 | G-anywhere |
| | motility protein 1 | | | NK | | |
| | (Protein ced-12 | | | | | |
| | homolog) | | | | | |
| 39 | Anaphase-promoting | APC5 | Q8BTZ4 | GRAMF | 740 | G-anywhere |
| | complex subunit 5 | | | LVSK | | |
| | (APC5) (Cyclosome | | | | | |
| | subunit 5) | | | | | |
| 40 | RNA-binding protein | RBM34 | Q8C5L7 | LNNSEL | 442 | G-anywhere |
| | 34 (RNA-binding | | | MGR | | |
| | motif protein 34) | | | | | |
| 41 | E3 ubiquitin-protein | ITCH | Q8C863 | ILNKPV | 864 | G-anywhere |
| | ligase Itchy (EC | | | GLK | | |
| | 2.3.2.26) (HECT-type | | | | | |
| | E3 ubiquitin | | | | | |
| | transferase Itchy | | | | | |
| | homolog) | | | | | |
| 42 | Coiled-coil domain- | CC159 | Q8C963 | WSTEQE | 411 | G-anywhere |
| | containing protein 159 | | | LYGAL | | |
| | | | | AQGLQ | | |
| | | | | GLQK | | |
| 43 | Death-inducer | DIDO1 | Q8C9B9 | SPAFEG | 2256 | G-anywhere |
| | obliterator 1 (DIO-1) | | | RQR | | |
| | (Death-associated | | | | | |
| | transcription factor 1) | | | | | |
| | (DATF-1) | | | | | |
| 44 | Coiled-coil domain- | CD158 | Q8CDI6 | ILRELD | 1109 | G-anywhere |
| | containing protein 158 | | | TEISFLK | | |
| | | | | GR | | |
| 45 | Structural maintenance | SMC4 | Q8CG47 | IFNLSG | 1286 | G-anywhere |
| | of chromosomes | | | GEK | | |
| | protein 4 (SMC protein | | | | | |
| | 4) (SMC-4) | | | | | |
| | (Chromosome- | | | | | |
| | associated polypeptide | | | | | |
| | C) (XCAP-C homolog) | | | | | |
| 46 | CD209 antigen-like | C209B | Q8CJ91 | IPISQGK | 325 | G-anywhere |
| | protein B (DC-SIGN- | | | | | |
| | related protein 1) (DC- | | | | | |
| | SIGNR1) (OtB7) (CD | | | | | |
| | antigen CD209) | | | | | |
| 47 | F-box DNA helicase 1 | FBH1 | Q8K219 | GINISNR | 1042 | G-anywhere |
| | (EC 3.6,4.12) (F-box | | | | | |
| | only protein 18) | | | | | |
| 48 | Serine dehydratase-like | SDSL | Q8R238 | IQLGCS | 329 | G-anywhere |
| | (EC 4.3.1.17) (L-serine | | | | | |
| | deaminase) (L-serine | | | | | |
| | dehydratase/L- | | | | | |
| | threonine deaminase) | | | | | |
| | (L-threonine | | | | | |
| | dehydratase) (TDH) | | | | | |
| | (EC 4.3.1.19)(SDH) | | | | | |
| 49 | Ribosome-releasing | RRF2M | Q8R2Q4 | ILYYSG | 779 | G-anywhere |
| | factor 2, mitochondrial | | | YTR | | |
| | (RRF2mt) (Elongation | | | | | |
| | factor G 2, | | | | | |
| | mitochondrial) (EF- | | | | | |
| | G2mt)(mEF-G 2) | | | | | |
| 50 | OTU domain- | OTU7A | Q8R554 | AAMQG | 926 | G-anywhere |
| | containing protein 7A | | | ER | | |
| | (EC 3.4.19.12) (Zinc | | | | | |
| | finger protein Cezanne | | | | | |
| | 2) | | | | | |
| 51 | Leucine-rich repeat- | LRC14 | Q8VC16 | ELSMGS | 493 | G-anywhere |
| | containing protein 14 | | | SLLSGR | | |
| 52 | Neurotrophin receptor- | NRIF2 | Q921B4 | NQQLGS | 824 | G-anywhere |
| | interacting factor 2 | | | EQGKT | | |
| | (Zinc finger protein | | | QTSR | | |
| | 369) | | | | | |
| 53 | Electron transfer | ETFD | Q921G7 | GIATND | 616 | G-anywhere |
| | flavoprotein- | | | VGIQK | | |
| | ubiquinone | | | | | |
| | oxidoreductase, | | | | | |
| | mitochondrial ETF- | | | | | |
| | QO) (ETF-ubiquinone | | | | | |
| | oxidoreductase) (EC | | | | | |
| | 1.5.5.1) (Electron- | | | | | |
| | transferring- | | | | | |
| | flavoprotein | | | | | |
| | dehydrogenase) (ETF | | | | | |
| | dehydrogenase) | | | | | |
| 54 | Polypeptide N- | GLT11 | Q921L8 | LMKCH | 608 | G-anywhere |
| | acetylgalactosaminyl- | | | GSGGSQ | | |
| | transferase 11 (EC | | | QWTFG | | |
| | 2.4.1.41) (Polypeptide | | | K | | |
| | GalNAc transferase | | | | | |
| | 11)(GalNAc-T11)(pp- | | | | | |
| | GaNTase 11)(Protein- | | | | | |
| | UDP | | | | | |
| | acetylgalactosaminyl- | | | | | |
| | transferase 11) (UDP- | | | | | |
| | GalNAc: polypeptide | | | | | |
| | N- | | | | | |
| | acetylgalactosaminyl- | | | | | |
| | transferase 11) | | | | | |
| 55 | TOM1-like protein 1 | TM1L1 | Q923U0 | LYKTGR | 474 | G-anywhere |
| | (Src-activating and | | | EMQER | | |
| | signaling molecule | | | | | |
| | protein) (Target of | | | | | |
| | Myb-like protein 1) | | | | | |
| 56 | Aconitate hydratase, | ACON | Q99KI0 | YLSKTG | 780 | G-anywhere |
| | mitochondrial | | | R | | |
| | (Aconitase) (EC | | | | | |
| | 4.2.1.3)(Citrate hydro- | | | | | |
| | lyase) | | | | | |
| 57 | Leucine-rich repeat- | LRC57 | Q9D1G5 | ELEGYD | 239 | G-anywhere |
| | containing protein 57 | | | K | | |
| 58 | Gamma- | GGCT | Q9D7X8 | LDFGNF | 188 | G-anywhere |
| | glutamylcyclotransferase | | | QGKMS | | |
| | (EC 4.3.2.9) | | | ER | | |
| 59 | Cyclin-L2 (Cyclin | CCNL2 | Q9JJA7 | ERTDNS | 518 | G-anywhere |
| | Ania-6b) (Paneth cell- | | | GKYK | | |
| | enhanced expression | | | | | |
| | protein)(PCEE) | | | | | |
| 60 | E3 SUMO-protein | PIAS4 | Q9JM05 | YLNGL | 507 | G-anywhere |
| | ligase PIAS4 (EC | | | GR | | |
| | 2.3.2.27) (PIASy) | | | | | |
| | (Protein inhibitor of | | | | | |
| | activated STAT protein | | | | | |
| | 4)(Protein inhibitor of | | | | | |
| | activated STAT protein | | | | | |
| | gamma) (PIAS- | | | | | |
| | gamma) (RING-type | | | | | |
| | E3 ubiquitin | | | | | |
| | transferase PIAS4) | | | | | |
| 61 | Calmodulin-4 | CALM4 | Q9JM83 | VADVD | 148 | G-anywhere |
| | (Calcium-binding | | | QDGK | | |
| | protein Dd112) | | | | | |
| 62 | PDZ domain- | PDZD4 | Q9QY39 | GCNMC | 772 | G-anywhere |
| | containing protein 4 | | | VVQK | | |
| | (PDZ domain- | | | | | |
| | containing RING | | | | | |
| | finger protein 4-like | | | | | |
| | protein) | | | | | |
| 63 | Short transient receptor | TRPC2 | Q9R244 | EGLTLP | 1172 | G-anywhere |
| | potential channel 2 | | | VPFNILP | | |
| | (TrpC2) (Transient | | | SPK | | |
| | receptor protein 2) | | | | | |
| | (TRP-2)(mTrp2) | | | | | |
| 64 | A-kinase anchor | AKA12 | Q9WTQ | ELEVPV | 1684 | G-anywhere |
| | protein 12 (AKAP-12) | | 5 | HTGPNS | | |
| | (Germ cell lineage | | | QKTAD | | |
| | protein gercelin) (Src- | | | LTR | | |
| | suppressed C kinase | | | | | |
| | substrate)(SSeCKS) | | | | | |
| 65 | ATP-dependent 6- | PFKAP | Q9WUA | GNQAV | 784 | G-anywhere |
| | phosphofructokinase, | | 3 | R | | |
| | platelet type (ATP- | | | | | |
| | PFK) (PFK-P) (EC | | | | | |
| | 2.7.1.11) (6- | | | | | |
| | phosphofructokinase | | | | | |
| | type C) | | | | | |
| | (Phosphofructo-1- | | | | | |
| | kinase isozyme C) | | | | | |
| | (PFK-C) | | | | | |
| | (Phosphohexokinase) | | | | | |
| 66 | Katanin p60 ATPase- | KTNA1 | Q9WV8 | GREEKN | 49. | G-anywhere |
| | containing subunit A1 | | 6 | K | | |
| | (Katanin p60 subunit | | | | | |
| | A1) (EC 5.6.1.1) | | | | | |
| | (Lipotransin) (p60 | | | | | |
| | katanin) | | | | | |
| 67 | R-spondin-1 (Cysteine- | RSPO1 | Q9Z132 | KGGQG | 265 | G-anywhere |
| | rich and single | | | R | | |
| | thrombospondin | | | | | |
| | domain-containing | | | | | |
| | protein 3) (Cristin-3) | | | | | |
| | (mCristin-3) (Roof | | | | | |
| | plate-specific spondin- | | | | | |
| | 1) | | | | | |
| 68 | V-type proton ATPase | VATC1 | Q9Z1G3 | ASAYN | 382 | G-anywhere |
| | subunit C 1 (V-ATPase | | | NLKGN | | |
| | subunit C 1)(Vacuolar | | | LQNLER | | |
| | proton pump subunit C | | | | | |
| | 1) | | | | | |

TABLE 3
A list of 54 protein candidates from RBCs modified with biotin-peptide on the side
chain of lysine(s).

| | | UniProt | Isoform | | | |
|---|---|---|---|---|---|---|
| No. | Protein names | ID | ID | Sequence | Length | Modifications |
| 1 | Extracellular calcium- | CASR | Q9QY96 | LFINEGK | 1079 | K-side chain |
| | sensing receptor | | | | | |
| | (CaSR) (Parathyroid | | | | | |
| | cell calcium-sensing | | | | | |
| | receptor) (PCaR1) | | | | | |
| 2 | Transcription factor | ZEP3 | A2A884 | GLPPMS | 2348 | K-side chain |
| | HIVEP3 (Human | | | VK | | |
| | immunodeficiency | | | | | |
| | virus type I enhancer- | | | | | |
| | binding protein 3 | | | | | |
| | homolog) (KB- | | | | | |
| | binding and | | | | | |
| | recognition | | | | | |
| | component)(Kappa-B | | | | | |
| | and V(D)J | | | | | |
| | recombination signal | | | | | |
| | sequences-binding | | | | | |
| | protein) (Kappa- | | | | | |
| | binding protein 1) | | | | | |
| | (KBP-1) | | | | | |
| | (Recombinant | | | | | |
| | component) | | | | | |
| | (Schnurri-3) (Zinc | | | | | |
| | finger protein ZAS3) | | | | | |
| 3 | Focadhesin | FOCAD | A2AKG | TYETNK | 1798 | K-side chain |
| | | | 8 | QPGLK | | |
| 4 | Arginine/serine-rich | RSRC2 | A2RTL5 | SQSAEI | 376 | K-side chain |
| | coiled-coil protein 2 | | | WEK | | |
| 5 | E3 ubiquitin-protein | RN213 | E9Q555 | EIDVQY | 5152 | K-side chain |
| | ligase RNF213 (EC | | | K | | |
| | 2.3.2.27)(EC 3.6.4.-) | | | | | |
| | (Mysterin) (RING | | | | | |
| | finger protein 213) | | | | | |
| | (RING-type E3 | | | | | |
| | ubiquitin transferase | | | | | |
| | RNF213) | | | | | |
| 6 | Brefeldin A-inhibited | BIG1 | G3X9K3 | FLTSQQL | 1846 | K-side chain |
| | guanine nucleotide- | | | FK | | |
| | exchange protein 1 | | | | | |
| | (BIG1)(Brefeldin A- | | | | | |
| | inhibited GEP 1) | | | | | |
| | (ADP-ribosylation | | | | | |
| | factor guanine | | | | | |
| | nucleotide-exchange | | | | | |
| | factor 1) | | | | | |
| 7 | Histone-lysine N- | NSD1 | O88491 | ETISAQ | 2588 | K-side chain |
| | methyltransferase, H3 | | | MVK | | |
| | lysine-36 and H4 | | | | | |
| | lysine-20 specific (EC | | | | | |
| | 2.1.1 .- ) (H3-K36- | | | | | |
| | HMTase) (H4-K20- | | | | | |
| | HMTase) (Nuclear | | | | | |
| | receptor-binding SET | | | | | |
| | domain-containing | | | | | |
| | protein 1) (NR- | | | | | |
| | binding SET domain- | | | | | |
| | containing protein) | | | | | |
| 7 | Histone-lysine N- | NSD1 | O88491 | LLNNMH | 2588 | K-side chain |
| | methyltransferase, H3 | | | EKTR | | |
| | lysine-36 and H4 | | | | | |
| | lysine-20 specific (EC | | | | | |
| | 2.1.1.-) (H3-K36- | | | | | |
| | HMTase) (H4-K20- | | | | | |
| | HMTase) (Nuclear | | | | | |
| | receptor-binding SET | | | | | |
| | domain-containing | | | | | |
| | protein 1) (NR- | | | | | |
| | binding SET domain- | | | | | |
| | containing protein) | | | | | |
| 8 | T-cell surface | CD3G | P11942 | NTWNLG | 182 | K-side chain |
| | glycoprotein CD3 | | | NNAK | | |
| | gamma chain (T-cell | | | | | |
| | receptor T3 gamma | | | | | |
| | chain) (CD antigen | | | | | |
| | CD3g) | | | | | |
| 9 | CD40 ligand (CD40- | CD40L | P27548 | KENSFE | 260 | K-side chain |
| | L) (T-cell antigen | | | MQR | | |
| | Gp39) (TNF-related | | | | | |
| | activation protein) | | | | | |
| | (TRAP) (Tumor | | | | | |
| | necrosis factor ligand | | | | | |
| | superfamily member | | | | | |
| | 5) (CD antigen | | | | | |
| | CD154) [Cleaved | | | | | |
| | into: CD40 ligand, | | | | | |
| | membrane form; | | | | | |
| | CD40 ligand, soluble | | | | | |
| | form (sCD40L)] | | | | | |
| 10 | Sulfotransferase 1E1 | STIE1 | P49891 | EGDVEK | 295 | K-side chain |
| | (STIE1)(EC 2.8.2.4) | | | CKEDAIF | | |
| | (Estrogen | | | NR | | |
| | sulfotransferase, testis | | | | | |
| | isoform) | | | | | |
| | (Sulfotransferase, | | | | | |
| | estrogen-preferring) | | | | | |
| 11 | Solute carrier family | S12A2 | P55012 | RQAMKE | 1205 | K-side chain |
| | 12 member 2 | | | MSIDQA | | |
| | (Basolateral Na-K-Cl | | | R | | |
| | symporter) | | | | | |
| | (Bumetanide-sensitive | | | | | |
| | sodium-(potassium)- | | | | | |
| | chloride cotransporter | | | | | |
| | 2) | | | | | |
| 12 | 26S proteasome | PRS10 | P62334 | ALQDYR | 389 | K-side chain |
| | regulatory subunit | | | KK | | |
| | 10B (26S proteasome | | | | | |
| | AAA-ATPase subunit | | | | | |
| | RPT4) (Proteasome | | | | | |
| | 26S subunit ATPase | | | | | |
| | 6) (Proteasome | | | | | |
| | subunit p42) | | | | | |
| 13 | Adenylate cyclase | ADCY6 | Q01341 | LLLSVLP | 1165 | K-side chain |
| | type 6 (EC 4.6.1.1) | | | QHVAME | | |
| | (ATP pyrophosphate- | | | MK | | |
| | lyase 6) (Adenylate | | | | | |
| | cyclase type VI) | | | | | |
| | (Adenylyl cyclase 6) | | | | | |
| | (AC6) (Ca(2+)- | | | | | |
| | inhibitable adenylyl | | | | | |
| | cyclase) | | | | | |
| 14 | Transcription factor | SOX13 | Q04891 | ILGSRW | 613 | K-side chain |
| | SOX-13 (SRY (Sex | | | KSMTNQ | | |
| | determining region | | | EK | | |
| | Y)-box 13)(mSox13) | | | | | |
| 15 | Leucine-rich repeat | LRIQ1 | Q0P5X1 | NQEKLM | 1673 | K-side chain |
| | and IQ domain- | | | AHKSEQ | | |
| | containing protein 1 | | | SR | | |
| 16 | von Willebrand factor | VWA3A | Q3UVV | EFQNDL | 1148 | K-side chain |
| | A domain-containing | | 9 | TGLIDEQ | | |
| | protein 3A | | | LSLKEK | | |
| 17 | Nesprin-3 (KASH | SYNE3 | Q4FZC9 | NQQLQR | 975 | K-side chain |
| | domain-containing | | | TEVDTG | | |
| | protein 3) (KASH3) | | | KK | | |
| | (Nuclear envelope | | | | | |
| | spectrin repeat protein | | | | | |
| | 3) | | | | | |
| 18 | Down syndrome cell | DSCL1 | Q4VA61 | DGQVIIS | 2053 | K-side chain |
| | adhesion molecule- | | | GSGVTIE | | |
| | like protein 1 | | | SK | | |
| | homolog | | | | | |
| 19 | Centrosome- | CP250 | Q60952 | QNEDYE | 2414 | K-side chain |
| | associated protein | | | KMVKAL | | |
| | CEP250 (250 kDa | | | R | | |
| | centrosomal protein) | | | | | |
| | (Cep250) | | | | | |
| | (Centrosomal Nek2- | | | | | |
| | associated protein 1) | | | | | |
| | (C-Nap1) | | | | | |
| | (Centrosomal protein | | | | | |
| | 2) (Intranuclear | | | | | |
| | matrix protein) | | | | | |
| 20 | Cytochrome b-245 | CY24B | Q61093 | TIELQM | 570 | K-side chain |
| | heavy chain (EC 1.-.-.-) | | | KK | | |
| | (CGD91-phox) | | | | | |
| | (Cytochrome b(558) | | | | | |
| | subunit beta) | | | | | |
| | (Cytochrome b558 | | | | | |
| | subunit beta) (Heme- | | | | | |
| | binding membrane | | | | | |
| | glycoprotein | | | | | |
| | gp91phox) | | | | | |
| | (Neutrophil | | | | | |
| | cytochrome b 91 kDa | | | | | |
| | polypeptide) (gp91-1) | | | | | |
| | (gp91-phox) (p22 | | | | | |
| | phagocyte B- | | | | | |
| | cytochrome) | | | | | |
| 21 | Heat shock protein | HS105 | Q61699 | NQQITH | 858 | K-side chain |
| | 105 kDa (42 degrees | | | ANNTVS | | |
| | C-HSP) (Heat shock | | | SFK | | |
| | 110 kDa protein) | | | | | |
| | (Heat shock-related | | | | | |
| | 100 kDa protein E7I) | | | | | |
| | (HSP-E7I) | | | | | |
| 22 | Tolloid-like protein 1 | TLL1 | Q62381 | LSEQSEK | 1013 | K-side chain |
| | (mTII)(EC 3.4.24.-) | | | NR | | |
| 23 | E3 ubiquitin-protein | SH3R1 | Q69ZI1 | LLSGAST | 892 | K-side chain |
| | ligase SH3RF1 (EC | | | KR | | |
| | 2.3.2.27) (Plenty of | | | | | |
| | SH3s) (Protein | | | | | |
| | POSH) (RING-type | | | | | |
| | E3 ubiquitin | | | | | |
| | transferase SH3RF1) | | | | | |
| | (SH3 domain- | | | | | |
| | containing RING | | | | | |
| | finger protein 1)(SH3 | | | | | |
| | multiple domains | | | | | |
| | protein 2) | | | | | |
| 24 | Tubulin epsilon and | TEDC2 | Q6GQV | VLGTRS | 436 | K-side chain |
| | delta complex protein | | 0 | TK | | |
| | 2 | | | | | |
| 25 | Vacuolar ATPase | VMA21 | Q78T54 | QWREGK | 101 | K-side chain |
| | assembly integral | | | QD | | |
| | membrane protein | | | | | |
| | Vma21 | | | | | |
| 26 | Centrosomal protein | CE120 | Q7TSG1 | DQQNNK | 988 | K-side chain |
| | of 120 kDa (Cep120) | | | PEIR | | |
| | (Coiled-coil domain- | | | | | |
| | containing protein | | | | | |
| | 100) | | | | | |
| 27 | Transcription | TAF1 | Q80UV9 | LKRNQE | 1891 | K-side chain |
| | initiation factor TFIID | | | K | | |
| | subunit 1 (EC | | | | | |
| | 2.3.1.48) (EC | | | | | |
| | 2.7.11.1) (Cell cycle | | | | | |
| | gene 1 protein)(TBP- | | | | | |
| | associated factor 250 | | | | | |
| | kDa) (p250) | | | | | |
| | (Transcription | | | | | |
| | initiation factor TFIID | | | | | |
| | 250 kDa subunit) | | | | | |
| | (TAF(II)250) (TAFII- | | | | | |
| | 250)(TAFII250) | | | | | |
| 28 | Carbohydrate | CHSTE | Q80V53 | LLSAYR | 376 | K-side chain |
| | sulfotransferase 14 | | | NK | | |
| | (EC 2.8.2.35) | | | | | |
| | (Dermatan 4- | | | | | |
| | sulfotransferase 1) | | | | | |
| | (D4ST-1) | | | | | |
| 29 | Multidrug resistance- | MRP9 | Q80WJ6 | LMNRFS | 1366 | K-side chain |
| | associated protein 9 | | | K | | |
| | (ATP-binding cassette | | | | | |
| | sub-family C member | | | | | |
| | 12) | | | | | |
| 30 | Uncharacterized | CJ062 | Q80Y39 | EMQRES | 304 | K-side chain |
| | protein C10orf62 | | | GK | | |
| | homolog | | | | | |
| 31 | Tenascin-N (TN-N) | TENN | Q80Z71 | LEEEMA | 1560 | K-side chain |
| | (Tenascin-W) (TN- | | | ELKEQC | | |
| | W) | | | NTNR | | |
| 32 | BRCA2-interacting | EMSY | Q8BMB | ITTIPMT | 1264 | K-side chain |
| | transcriptional | | 0 | SK | | |
| | repressor EMSY | | | | | |
| 33 | Zinc finger protein | DZIP1 | Q8BMD | LNKKTS | 852 | K-side chain |
| | DZIP1 (DAZ- | | 2 | LR | | |
| | interacting protein 1 | | | | | |
| | homolog) | | | | | |
| 34 | Phosphatidylinositol | PK3CB | Q8BTI9 | KMYEQE | 1064 | K-side chain |
| | 4,5-bisphosphate 3- | | | MIAIEAA | | |
| | kinase catalytic | | | INR | | |
| | subunit beta isoform | | | | | |
| | (PI3-kinase subunit | | | | | |
| | beta) (PI3K-beta) | | | | | |
| | (PI3Kbeta) (PtdIns-3- | | | | | |
| | kinase subunit beta) | | | | | |
| | (EC 2.7.1.153) | | | | | |
| | (Phosphatidylinositol | | | | | |
| | 4,5-bisphosphate 3- | | | | | |
| | kinase 110 kDa | | | | | |
| | catalytic subunit beta) | | | | | |
| | (PtdIns-3-kinase | | | | | |
| | subunit p110-beta) | | | | | |
| | (p110beta) | | | | | |
| 35 | Dynein heavy chain 3, | DYH3 | Q8BW9 | KMKFNL | 4083 | K-side chain |
| | axonemal (Axonemal | | 4 | K | | |
| | beta dynein heavy | | | | | |
| | chain 3) (Ciliary | | | | | |
| | dynein heavy chain 3) | | | | | |
| 36 | E3 ubiquitin-protein | ITCH | Q8C863 | ILNKPVG | 864 | K-side chain |
| | ligase Itchy (EC | | | LK | | |
| | 2.3.2.26) (HECT-type | | | | | |
| | E3 ubiquitin | | | | | |
| | transferase Itchy | | | | | |
| | homolog) | | | | | |
| 37 | MICOS complex | MIC60 | Q8CAQ | LEEKRTF | 757 | K-side chain |
| | subunit Mic60 | | 8 | DSAVAK | | |
| | (Mitochondrial inner | | | | | |
| | membrane protein) | | | | | |
| | (Mitofilin) | | | | | |
| 38 | Ras-related protein | RAB44 | Q8CB87 | VKNLLV | 973 | K-side chain |
| | Rab-44 | | | DNK | | |
| 39 | Leucine-rich repeat- | LRRC9 | Q8CDN | IEFLQQK | 1456 | K-side chain |
| | containing protein 9 | | 9 | | | |
| 40 | Structural | SMC4 | Q8CG47 | IFNLSGG | 1286 | K-side chain |
| | maintenance of | | | EK | | |
| | chromosomes protein | | | | | |
| | 4 (SMC protein 4) | | | | | |
| | (SMC-4) | | | | | |
| | (Chromosome- | | | | | |
| | associated | | | | | |
| | polypeptide C | | | | | |
| | (XCAP-C homolog) | | | | | |
| 41 | CD209 antigen-like | C209B | Q8CJ91 | IPISQGK | 325 | K-side chain |
| | protein B (DC-SIGN- | | | | | |
| | related protein 1) | | | | | |
| | (DC-SIGNR1)(OtB7) | | | | | |
| | (CD antigen CD209) | | | | | |
| 42 | F-box DNA helicase | FBH1 | Q8K219 | YVTAAE | 1042 | K-side chain |
| | 1 (EC 3.6.4.12) (F- | | | DKELEA | | |
| | box only protein 18) | | | KIAVVE | | |
| | | | | K | | |
| 43 | Major intrinsically | MNAR1 | Q8K3V7 | CSVNNQ | 917 | K-side chain |
| | disordered Notch2- | | | QSK | | |
| | binding receptor 1 | | | | | |
| | (Membrane integral | | | | | |
| | NOTCH2-associated | | | | | |
| | receptor 1) (Protein | | | | | |
| | DD1) (Ubiquitination | | | | | |
| | and mTOR signaling | | | | | |
| | protein) | | | | | |
| 44 | Actin-related protein | ARP8 | Q8R2S9 | QNGLKM | 624 | K-side chain |
| | 8 | | | VDQAIW | | |
| | | | | SK | | |
| 45 | Leucine-rich repeat- | LRC14 | Q8VC16 | VAFMDK | 493 | K-side chain |
| | containing protein 14 | | | KTLVLR | | |
| 46 | Electron transfer | ETFD | Q921G7 | GIATND | 616 | K-side chain |
| | flavoprotein- | | | VGIQK | | |
| | ubiquinone | | | | | |
| | oxidoreductase, | | | | | |
| | mitochondrial (ETF- | | | | | |
| | QO)(ETF-ubiquinone | | | | | |
| | oxidoreductase) (EC | | | | | |
| | 1.5.5.1) (Electron- | | | | | |
| | transferring- | | | | | |
| | flavoprotein | | | | | |
| | dehydrogenase) (ETF | | | | | |
| | dehydrogenase) | | | | | |
| 47 | Tetratricopeptide | TTC14 | Q9CSP9 | NEAPEE | 761 | K-side chain |
| | repeat protein 14 | | | MLNSK | | |
| | (TPR repeat protein | | | | | |
| | 14) | | | | | |
| 48 | Gamma- | GGCT | Q9D7X8 | LDFGNF | 188 | K-side chain |
| | glutamylcyclotransferase | | | QGKMSE | | |
| | (EC 4.3.2.9) | | | R | | |
| 49 | LanC-like protein 2 | LANC2 | Q9JJK2 | SLSREER | 450 | K-side chain |
| | (Testis-specific | | | K | | |
| | adriamycin sensitivity | | | | | |
| | protein) | | | | | |
| 50 | Calmodulin-4 | CALM4 | Q9JM83 | VADVDQ | 148 | K-side chain |
| | (Calcium-binding | | | DGK | | |
| | protein Dd112) | | | | | |
| 51 | Plexin-C1 (Virus- | PLXC1 | Q9QZC2 | NQELCQ | 1574 | K-side chain |
| | encoded semaphorin | | | VAVEKS | | |
| | protein receptor) (CD | | | PK | | |
| | antigen CD232) | | | | | |
| 52 | Protein BEX1 (Brain- | BEX1 | Q9R224 | NLNMEN | 128 | K-side chain |
| | expressed X-linked | | | DHQKKE | | |
| | protein 1 homolog) | | | EK | | |
| | (Reduced expression | | | | | |
| | protein 3)(REX-3) | | | | | |
| 53 | Short transient | TRPC2 | Q9R244 | EGLTLP | 1172 | K-side chain |
| | receptor potential | | | VPFNILP | | |
| | channel 2 (TrpC2) | | | SPK | | |
| | (Transient receptor | | | | | |
| | protein 2) (TRP-2) | | | | | |
| | (mTrp2) | | | | | |
| 54 | A-kinase anchor | AKA12 | Q9WTQ | ELEVPV | 1684 | K-side chain |
| | protein 12 (AKAP- | | 5 | HTGPNS | | |
| | 12) (Germ cell | | | QKTADL | | |
| | lincage protein | | | TR | | |
| | gercelin) (Src- | | | | | |
| | suppressed C kinase | | | | | |
| | substrate)(SSeCKS) | | | | | |

TABLE 4
A list of 18 protein candidates from RBCs modified with biotin-peptide
on glycine and the side chain of lysine.

| | | UniProt | Isoform | | | |
|---|---|---|---|---|---|---|
| No. | Protein names | ID | ID | Sequence | Length | Modifications |
| 1 | Extracellular calcium- | CASR | Q9QY96 | LFINEGK | 1079 | G-anywhere |
| | sensing receptor | | | | | and K side |
| | (CaSR) (Parathyroid | | | | | chain |
| | cell calcium-sensing | | | | | |
| | receptor)(PCaR1) | | | | | |
| 2 | T-cell surface | CD3G | P11942 | NTWNLG | 182 | G-anywhere |
| | glycoprotein CD3 | | | NNAK | | and K side |
| | gamma chain (T-cell | | | | | chain |
| | receptor T3 gamma | | | | | |
| | chain) (CD antigen | | | | | |
| | CD3g) | | | | | |
| 3 | Sulfotransferase 1E1 | STIE1 | P49891 | EGDVEK | 295 | G-anywhere |
| | (STIE1) (EC 2.8.2.4) | | | CKEDAIF | | and K side |
| | (Estrogen | | | NR | | chain |
| | sulfotransferase, testis | | | | | |
| | isoform) | | | | | |
| | (Sulfotransferase, | | | | | |
| | estrogen-preferring) | | | | | |
| 4 | Down syndrome cell | DSCL1 | Q4VA61 | DGQVIIS | 2053 | G-anywhere |
| | adhesion molecule- | | | GSGVTIE | | and K side |
| | like protein 1 | | | SK | | chain |
| | homolog | | | | | |
| 5 | E3 ubiquitin-protein | SH3R1 | Q69ZI1 | LLSGAST | 892 | G-anywhere |
| | ligase SH3RF1 (EC | | | KR | | and K side |
| | 2.3.2.27) (Plenty of | | | | | chain |
| | SH3s) (Protein | | | | | |
| | POSH) (RING-type | | | | | |
| | E3 ubiquitin | | | | | |
| | transferase SH3RF1) | | | | | |
| | (SH3 domain- | | | | | |
| | containing RING | | | | | |
| | finger protein 1) (SH3 | | | | | |
| | multiple domains | | | | | |
| | protein 2) | | | | | |
| 6 | Tubulin epsilon and | TEDC2 | Q6GQV | VLGTRS | 436 | G-anywhere |
| | delta complex protein | | 0 | TK | | and K side |
| | 2 | | | | | chain |
| 7 | Vacuolar ATPase | VMA21 | Q78T54 | QWREGK | 101 | G-anywhere |
| | assembly integral | | | QD | | and K side |
| | membrane protein | | | | | chain |
| | Vma21 | | | | | |
| 8 | Uncharacterized | CJ062 | Q80Y39 | EMQRES | 304 | G-anywhere |
| | protein C10orf62 | | | GK | | and K side |
| | homolog | | | | | chain |
| 9 | E3 ubiquitin-protein | ITCH | Q8C863 | ILNKPVG | 864 | G-anywhere |
| | ligase Itchy (EC | | | LK | | and K side |
| | 2.3.2.26) (HECT-type | | | | | chain |
| | E3 ubiquitin | | | | | |
| | transferase Itchy | | | | | |
| | homolog) | | | | | |
| 10 | Structural | SMC4 | Q8CG47 | IFNLSGG | 1286 | G-anywhere |
| | maintenance of | | | EK | | and K side |
| | chromosomes protein | | | | | chain |
| | 4 (SMC protein 4) | | | | | |
| | (SMC-4) | | | | | |
| | (Chromosome- | | | | | |
| | associated | | | | | |
| | polypeptide | | | | | |
| | (XCAP-C homolog) | | | | | |
| 11 | CD209 antigen-like | C209B | Q8CJ91 | IPISQGK | 325 | G-anywhere |
| | protein B (DC-SIGN- | | | | | and K side |
| | related protein 1) | | | | | chain |
| | (DC-SIGNR1)(OtB7) | | | | | |
| | (CD antigen CD209) | | | | | |
| 12 | F-box DNA helicase | FBH1 | Q8K219 | GINISNR; | 1042 | G-anywhere |
| | 1 (EC 3.6.4.12) (F- | | | and | | and K side |
| | box only protein 18) | | | YVTAAE | | chain |
| | | | | DKELEA | | |
| | | | | KIAVVE | | |
| | | | | K | | |
| 13 | Leucine-rich repeat- | LRC14 | Q8VC16 | ELSMGS | 493 | G-anywhere |
| | containing protein 14 | | | SLLSGR; | | and K side |
| | | | | and | | chain |
| | | | | VAFMDK | | |
| | | | | KTLVLR | | |
| 14 | Electron transfer | ETFD | Q921G7 | GIATND | 616 | G-anywhere |
| | flavoprotein- | | | VGIQK | | and K side |
| | ubiquinone | | | | | chain |
| | oxidoreductase, | | | | | |
| | mitochondrial (ETF- | | | | | |
| | QO)(ETF-ubiquinone | | | | | |
| | oxidoreductase) (EC | | | | | |
| | 1.5.5.1) (Electron- | | | | | |
| | transferring- | | | | | |
| | flavoprotein | | | | | |
| | dehydrogenase) (ETF | | | | | |
| | dehydrogenase) | | | | | |
| 15 | Gamma- | GGCT | Q9D7X8 | LDFGNF | 188 | G-anywhere |
| | glutamylcyclotransferase | | | QGKMSE | | and K side |
| | (EC 4.3.2.9) | | | R | | chain |
| 16 | Calmodulin-4 | CALM4 | Q9JM83 | VADVDQ | 148 | G-anywhere |
| | (Calcium-binding | | | DGK | | and K side |
| | protein Dd112) | | | | | chain |
| 17 | Short transient | TRPC2 | Q9R244 | EGLTLP | 1172 | G-anywhere |
| | receptor potential | | | VPFNILP | | and K side |
| | channel 2 (TrpC2) | | | SPK | | chain |
| | (Transient receptor | | | | | |
| | protein 2) (TRP-2) | | | | | |
| | (mTrp2) | | | | | |
| 18 | A-kinase anchor | AKA12 | Q9WTQ | ELEVPV | 1684 | G-anywhere |
| | protein 12 (AKAP- | | 5 | HTGPNS | | and K side |
| | 12) (Germ cell | | | QKTADL | | chain |
| | lineage protein | | | TR | | |
| | gercelin) (Src- | | | | | |
| | suppressed C kinase | | | | | |
| | substrate)(SSeCKS) | | | | | |

A list of 22 membrane protein candidates from RBCs modified with biotin-peptide on glycine and the side chain of lysine were shown in Table 5.

TABLE 5

| | | | | | | Modification |
|---|---|---|---|---|---|---|
| | | UniProt | Isoform | | | type and |
| No. | Protein names | ID | ID | Sequence | Length | position |
| 1 | Extracellular calcium- | CASR | Q9QY96 | LFINEGK | 1079 | G-anywhere |
| | sensing receptor | | | | | and K side |
| | (CaSR)(Parathyroid | | | | | chain; G526 / |
| | cell calcium-sensing | | | | | K527 |
| | receptor)(PCaR1) | | | | | |
| 2 | T-cell surface | CD3G | P11942 | NTWNLG | 182 | G-anywhere |
| | glycoprotein CD3 | | | NNAK | | and K side |
| | gamma chain (T-cell | | | | | chain; G158 / |
| | receptor T3 gamma | | | | | K162 |
| | chain)(CD antigen | | | | | |
| | CD3g) | | | | | |
| 3 | Down syndrome cell | DSCL1 | Q4VA61 | DGQVIIS | 2053 | G-anywhere |
| | adhesion molecule- | | | GSGVTIE | | and K side |
| | like protein 1 | | | SK | | chain; G698 / |
| | homolog | | | | | K706 |
| 4 | Short transient | TRPC2 | Q9R244 | EGLTLP | 1172 | G-anywhere |
| | receptor potential | | | VPFNILP | | and K side |
| | channel 2 (TrpC2) | | | SPK | | chain; G950 / |
| | (Transient receptor | | | | | K964 |
| | protein 2)(TRP-2) | | | | | |
| | (mTrp2) | | | | | |
| 5 | CD209 antigen-like | C209B | Q8CJ91 | IPISQGK | 325 | G-anywhere |
| | protein B (DC-SIGN- | | | | | and K side |
| | related protein 1) | | | | | chain; G110 / |
| | (DC-SIGNR1)(OtB7) | | | | | K111 |
| | (CD antigen CD209) | | | | | |
| 6 | E3 ubiquitin-protein | ITCH | Q8C863 | ILNKPVG | 864 | G-anywhere |
| | ligase Itchy (EC | | | LK | | and K side |
| | 2.3.2.26)(HECT-type | | | | | chain; K631 / |
| | E3 ubiquitin | | | | | G634 |
| | transferase Itchy | | | | | |
| | homolog) | | | | | |
| 7 | A-kinase anchor | AKA12 | Q9WTQ | ELEVPV | 1684 | G-anywhere |
| | protein 12 (AKAP-12) | | 5 | HTGPNS | | and K side |
| | (Germ cell lineage | | | QKTADL | | chain; G1259/ |
| | protein gercelin)(Src- | | | TR | | K1264 |
| | suppressed C kinase | | | | | |
| | substrate)(SSeCKS) | | | | | |
| 8 | Inter alpha-trypsin | ITIH4 | A6X935 | GSRSQIP | 942 | G-anywhere; |
| | inhibitor, heavy chain | | | R | | G642 |
| | 4 (ITI heavy chain | | | | | |
| | H4)(ITI-HC4)(Inter- | | | | | |
| | alpha-inhibitor heavy | | | | | |
| | chain 4) | | | | | |
| 9 | Potassium- | ATP4A | Q64436 | ILSAQGC | 1033 | G-anywhere; |
| | transporting ATPase | | | K | | G219 |
| | alpha chain 1 (EC | | | | | |
| | 7.2.2.19)(Gastric | | | | | |
| | H(+)/K(+) ATPase | | | | | |
| | subunit alpha)(Proton | | | | | |
| | pump) | | | | | |
| 10 | P2X purinoceptor 1 | P2RX1 | P51576 | NLSPGF | 399 | G-anywhere; |
| | (P2X1)(ATP | | | NFR | | G288 |
| | receptor)(Purinergic | | | | | |
| | receptor) | | | | | |
| 11 | Ryanodine receptor 3 | RYR3 | A2AGL3 | NYMMS | 4863 | G-anywhere; |
| | (RYR-3)(RyR3) | | | NGYK | | G962 |
| | (Brain ryanodine | | | | | |
| | receptor-calcium | | | | | |
| | release channel) | | | | | |
| | (Brain-type ryanodine | | | | | |
| | receptor)(Type 3 | | | | | |
| | ryanodine receptor) | | | | | |
| 12 | Scavenger receptor | C163A | Q2VLH6 | FQGKWG | 1121 | G-anywhere; |
| | cysteine-rich type 1 | | | TVCDDN | | G180 |
| | protein M130 (CD | | | FSK | | |
| | antigen CD163) | | | | | |
| | [Cleaved into: Soluble | | | | | |
| | CD163 (sCD163)] | | | | | |
| 13 | APC membrane | AMER1 | Q7TS75 | LFGGKK | 1132 | G-anywhere; |
| | recruitment protein 1 | | | | | G61 |
| | (Amer1)(Protein | | | | | |
| | FAM123B) | | | | | |
| 14 | Serine/threonine- | MRCKB | Q7TT50 | DIKPDN | 1713 | G-anywhere; |
| | protein kinase MRCK | | | VLLDVN | | G212 |
| | beta (EC 2.7.11.1) | | | GHIR | | |
| | (CDC42-binding | | | | | |
| | protein kinase beta) | | | | | |
| | (DMPK-like beta) | | | | | |
| | (Myotonic dystrophy | | | | | |
| | kinase-related | | | | | |
| | CDC42-binding | | | | | |
| | kinase beta)(MRCK | | | | | |
| | beta)(Myotonic | | | | | |
| | dystrophy protein | | | | | |
| | kinase-like beta) | | | | | |
| 15 | Engulfment and cell | ELMO1 | Q8BPU7 | GALKQN | 727 | G-anywhere; |
| | motility protein 1 | | | K | | G629 |
| | (Protein ced-12 | | | | | |
| | homolog) | | | | | |
| 16 | Desmoplakin (DP) | DESP | E9Q557 | NSQGSE | 2883 | G-anywhere; |
| | | | | MFGDDD | | G608 |
| | | | | KRR | | |
| 17 | CD40 ligand (CD40- | CD40L | P27548 | KENSFE | 260 | K-side chain; |
| | L)(T-cell antigen | | | MQR | | K106 |
| | Gp39)(TNF-related | | | | | |
| | activation protein) | | | | | |
| | (TRAP)(Tumor | | | | | |
| | necrosis factor ligand | | | | | |
| | superfamily member | | | | | |
| | 5)(CD antigen | | | | | |
| | CD154)[Cleaved | | | | | |
| | into: CD40 ligand, | | | | | |
| | membrane form; | | | | | |
| | CD40 ligand, soluble | | | | | |
| | form (sCD40L)] | | | | | |
| 18 | Solute carrier family | S12A2 | P55012 | RQAMKE | 1205 | K-side chain; |
| | 12 member 2 | | | MSIDQA | | K826 |
| | (Basolateral Na-K-Cl | | | R | | |
| | symporter) | | | | | |
| | (Bumetanide-sensitive | | | | | |
| | sodium-(potassium)- | | | | | |
| | chloride cotransporter | | | | | |
| | 2) | | | | | |
| 19 | Adenylate cyclase | ADCY6 | Q01341 | LLLSVLP | 1165 | K-side chain; |
| | type 6 (EC 4.6.1.1) | | | QHVAME | | K353 |
| | (ATP pyrophosphate- | | | MK | | |
| | lyase 6)(Adenylate | | | | | |
| | cyclase type VI) | | | | | |
| | (Adenylyl cyclase 6) | | | | | |
| | (AC6)(Ca(2+)- | | | | | |
| | inhibitable adenylyl | | | | | |
| | cyclase) | | | | | |
| 20 | Cytochrome b-245 | CY24B | Q61093 | TIELQM | 570 | K-side chain; |
| | heavy chain (EC 1.-.-.-) | | | KK | | K313 / K314 |
| | (CGD91-phox) | | | | | |
| | (Cytochrome b(558) | | | | | |
| | subunit beta) | | | | | |
| | (Cytochrome b558 | | | | | |
| | subunit beta)(Heme- | | | | | |
| | binding membrane | | | | | |
| | glycoprotein | | | | | |
| | gp91phox) | | | | | |
| | (Neutrophil | | | | | |
| | cytochrome b 91 kDa | | | | | |
| | polypeptide)(gp91-1) | | | | | |
| | (gp91-phox)(p22 | | | | | |
| | phagocyte B- | | | | | |
| | cytochrome) | | | | | |
| 21 | Major intrinsically | MNAR1 | Q8K3V7 | CSVNNQ | 917 | K-side chain; |
| | disordered Notch2- | | | QSK | | K79 |
| | binding receptor 1 | | | | | |
| | (Membrane integral | | | | | |
| | NOTCH2-associated | | | | | |
| | receptor 1)(Protein | | | | | |
| | DD1)(Ubiquitination | | | | | |
| | and mTOR signaling | | | | | |
| | protein) | | | | | |
| 22 | Plexin-C1 (Virus- | PLXC1 | Q9QZC2 | NQELCQ | 1574 | K-side chain; |
| | encoded semaphorin | | | VAVEKS | | K642 |
| | protein receptor)(CD | | | PK | | |
| | antigen CD232) | | | | | |

### Example 2. Mg SrtA-Mediated Protein-Cell Conjugation Via Irreversible Linker

### Methods

Recombinant Protein Expression and Purification in E. coli

Mg SrtA and eGFP-cys cDNA(SEQ ID NO: 37/38) were cloned in pET vectors and transformed in E. coli BL2I(DE3) cells for protein expression. Transformed cells were cultured at 37° C. until the OD₆₀₀ reached 0.6-0.8, and then 500 μM IPTG was added. The cells were cultured with IPTG for 4 hrs at 37° C. until harvested by centrifugation and subjected to lysis by precooled lysis buffer (20 mM Tris-HCl, pH 7.8, 500 mM NaCl. The lysates were sonicated on ice (5s on, 5s off, 60 cycles, 25% power, Branson Sonifier 550 Ultrasonic Cell Disrupter). All supernatants were filtered by 0.45 μM filter after centrifugation at 14,000 g for 40 min at 4° C. Filtered supernatants were loaded onto HisTrap FF 1 mL column (GE Healthcare) connected to the AKTA design chromatography systems. The proteins were eluted with the elution buffer containing 20 mM Tris-HCl, pH 7.8, 500 mM NaCl and 300 mM imidazole. All eluted fractions were analyzed on an SDS-PAGE gel.

The amino acid sequence of eGFP-Cys is as shown in SEQ ID NO: 37 below:

MHHHHHHMVSKGEELFTGVVPILVELDGDVNGHKESVSGEGEGDATYGKL
TLKFICTTGKLPVPWPTLVTTLTYGVQCFSRYPDHMKQHDFFKSAMPEGY
VQERTIFFKDDGNYKTRAEVKFEGDTLVNRIELKGIDFKEDGNILGHKLE
YNYNSHNVYIMADKQKNGIKVNFKIRHNIEDGSVQLADHYQQNTPIGDGP
VLLPDNHYLSTQSALSKDPNEKRDHMVLLEFVTAAGITLGMDELYKC*

The nucleotide sequence of eGFP-Cys is as shown in SEQ ID NO: 38 below:

ATGCACCACCACCACCACCACATGGTTAGTAAAGGGGAAGAATTATTTAC
CGGCGTGGTGCCGATTCTGGTTGAACTGGACGGCGACGTGAACGGCCACA
AATTCAGCGTTAGCGGCGAGGGCGAAGGTGACGCGACCTACGGCAAGCTG
ACCCTGAAATTTATCTGCACCACCGGCAAGCTGCCGGTGCCGTGGCCGAC
CCTGGTTACCACCCTGACCTACGGTGTTCAGTGCTTCAGCCGTTATCCGG
ACCACATGAAGCAACACGATTTCTTTAAAAGCGCGATGCCGGAGGGCTAC
GTGCAGGAACGTACCATCTTCTTTAAGGACGATGGTAACTATAAAACCCG
TGCGGAAGTGAAGTTCGAAGGCGACACCCTGGTTAACCGTATCGAGCTGA
AGGGTATTGACTTTAAAGAAGATGGCAACATTCTGGGTCACAAACTGGAG
TACAACTATAACAGCCACAACGTGTATATCATGGCGGATAAGCAGAAAAA
CGGCATTAAGGTTAACTTCAAAATCCGTCACAACATTGAAGACGGTAGCG
TGCAACTGGCGGATCACTACCAGCAAAACACCCCGATTGGCGACGGTCCG
GTTCTGCTGCCGGATAACCACTATCTGAGCACCCAAAGCGCGCTGAGCAA
GGACCCGAACGAGAAACGTGATCACATGGTGCTGCTGGAATTTGTTACCGA
CGGCGGGTATCACCCTGGGTATGGACGAACTGTATAAGTGTTA

### Irreversible Linker Conjugation to Protein by Cysteine Coupling

Irreversible linker, 6-Maleimidohexanoic Acid-Leu-Pro-Glu-Thr-2-hydroxyacetic acid-Gly (6-Maleimnidohexanoic Acid-LPET-(2-hydroxyacetic acid)-G, 6-Mal-LPET*G), was synthesized with more than 99% purity. Reactions were performed in a total volume of 1 mL at room temperature for 1 hr in PBS buffer while being rotated at a speed of 10 rpm. The concentrations of 6-Mal-LPET*G and eGFP-cys protein were 2 mM and 500 AM, respectively. This method uses a four-fold molar excess of irreversible linker to eGFP-cys protein. After the reaction, the eGFP-cys-6-Mal-LPET*G products were collected by removal of excess irreversible linker via dialysis and ultrafiltration.

### Mg SrtA-Mediated Enzymatic Labeling of Membrane Proteins

Reactions were performed in a total volume of 200 μL at 37° C. for 2 hrs in PBS buffer while being rotated at a speed of 10 rpm. The concentration of mg SrtA was 10 μM and the eGFP-cys-6-Mal-LPET*G substrates were in the range of 25-75 μM. Human or mouse RBCs were washed twice with PBS before the enzymatic reaction. The concentration of RBCs in the reaction was 1×10⁹/mL. After the reaction, the labeling efficiency of RBCs was analyzed by Beckman Coulter CytoFLEX LX or Merck Amnis Image Stream MarkII.

### Product Identified by Mass Spectrometry.

Chromatographic desalting and separation of proteins were performed on the 1260 Infinity II system (Agilent Technologies) equipped with a ZORBAX 300SB-C3 column (2.1×150 mm) (Agilent Technologies). 1 μg protein was loaded onto the column and separated from the interference species with a gradient of mobile phase A (water, 0.1% formic acid) and mobile phase B (acetonitrile, 0.08% formic acid) at a flow rate of 0.4 ml/min. The gradient was 5%-95% phase B in 12 min. Following chromatographic separation, the protein samples were analyzed on a 6230 TOF LC/MS spectrometer (Agilent Technologies) equipped with a Dual ESI ion source. TOF-MS spectra were extracted from the total ion chromatograms (TICs) and deconvoluted using the Maximum Entropy incorporated in BioConfirm 10.0 software (Agilent Technologies).

### In-Gel Digestion

The whole gel was stained by Coomassie blue (H₂O, 0.1% w/v Coomassie brilliant blue R250, 40% v/v methanol and 10% v/v acetic acid) at room temperature with gentle shaking overnight, and then destained with the destaining solution (40% v/v methanol and 10% v/v acetic acid in water). The gel was rehydrated three times in distilled water at room temperature for 10 min with gentle agitation. The protein bands were cut out and further cut off into ca 1×1 mm² pieces, followed by reduction with 10 mM TCEP in 25 mM NH4HCO3 at 25° C. for 30 min, alkylation with 55 mM IAA in 25 mM NH₄HCO₃ solution at 25° C. in the dark for 30 min, sequential digestion with rPNGase F at a concentration of 100 unit/ml at 37° C. for 4 hrs, and digestions with trypsin at a concentration of 12.5 ng/mL at 37° C. overnight (1st digestion for 4 hrs and 2nd digestion for 12 hrs). Tryptic peptides were then extracted out from gel pieces by using 50% ACN/2.5% FA for three times and the peptide solution was dried under vacuum. Dry peptides were purified by Pierce C18 Spin Tips (Thermo Fisher, USA).

### Results

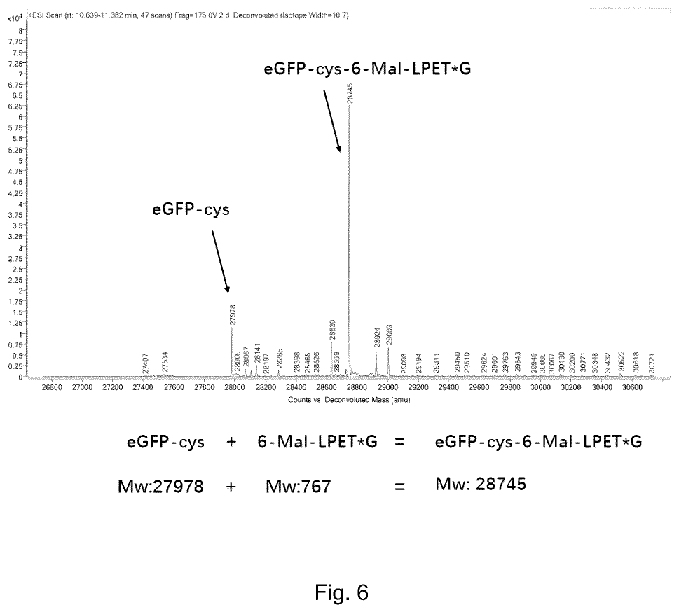

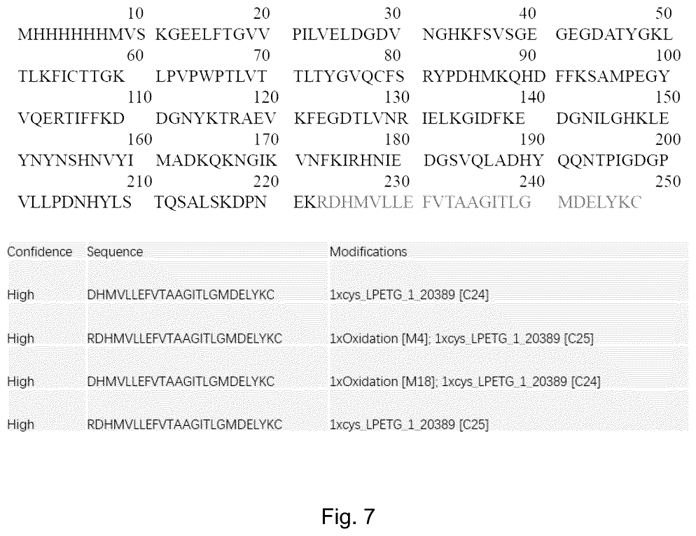

We first characterized the irreversible linker for protein conjugation. eGFP was used to test the conjugation efficiency of the reaction. We expressed and purified the eGFP with cysteine at the C terminus (eGFP-cys). We also synthesized the irreversible linker, 6-Mal-LPET*G. These two reaction substrates were mixed at a ratio of 1:4=eGFP-cys:6-Mal-LPET*G for reaction (FIG. 4). The final product of the reaction was collected for identification by mass spectrometry. The results showed that the molecular weight of the reaction product is the sum of the reaction substrate and the irreversible linker (FIG. 6). The C-terminal cysteine is exposed for the reaction, according to the structural analysis of eGFP. In order to further verify whether the reaction occurred on the sulfhydryl group of the C-terminal cysteine, we performed tandem mass spectrometry. The results showed that all modifications were on the C-terminal cysteine (FIG. 7).

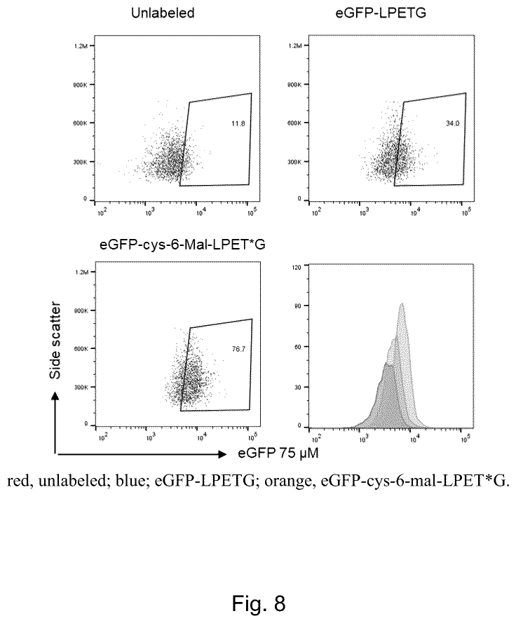

Then we characterized the labeling efficacy of different kinds of eGFP on the RBC membrane. eGFP-LPETG was employed as the control of the reversible substrate. Our results showed that >75% of natural RBCs were eGFP-cys-6-Mal-LPET*G-labeled by mg SrtA in vitro. In contrast, only about 30% of the signal was detected on the surface of RBCs by using reversible substrate eGFP-LPETG (FIG. 8).

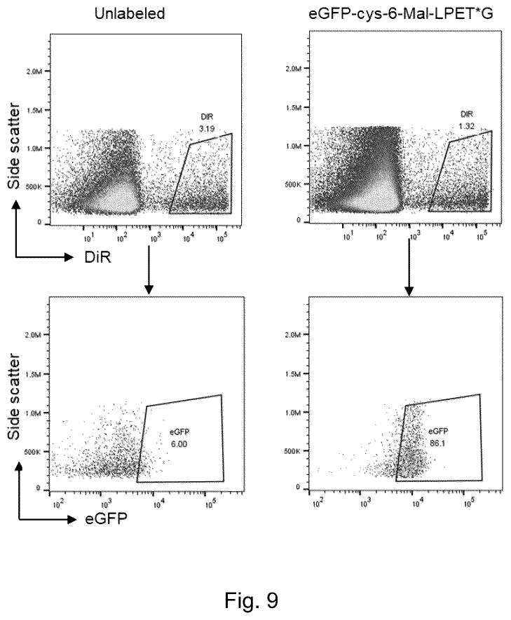

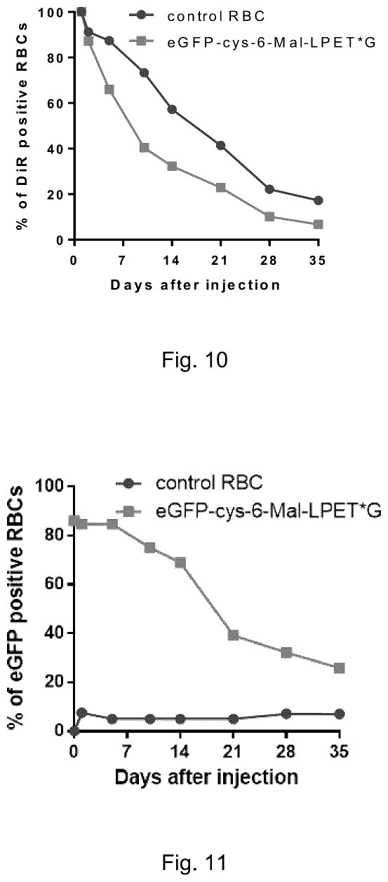

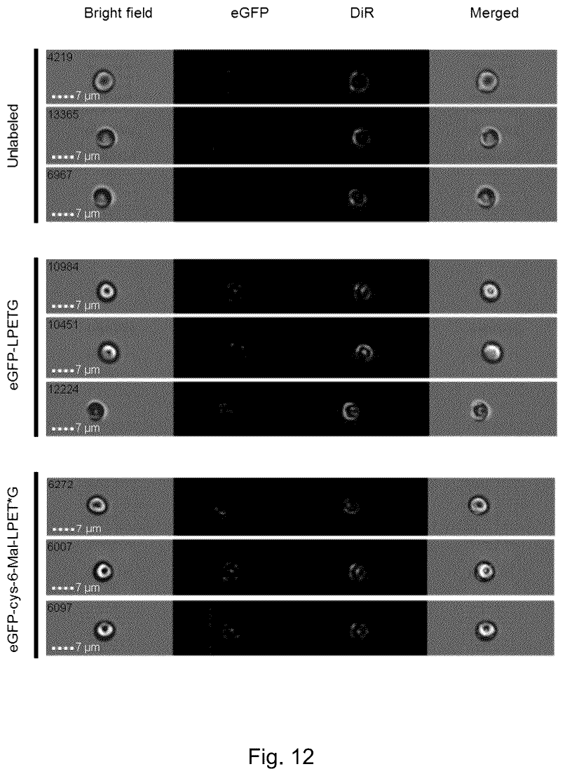

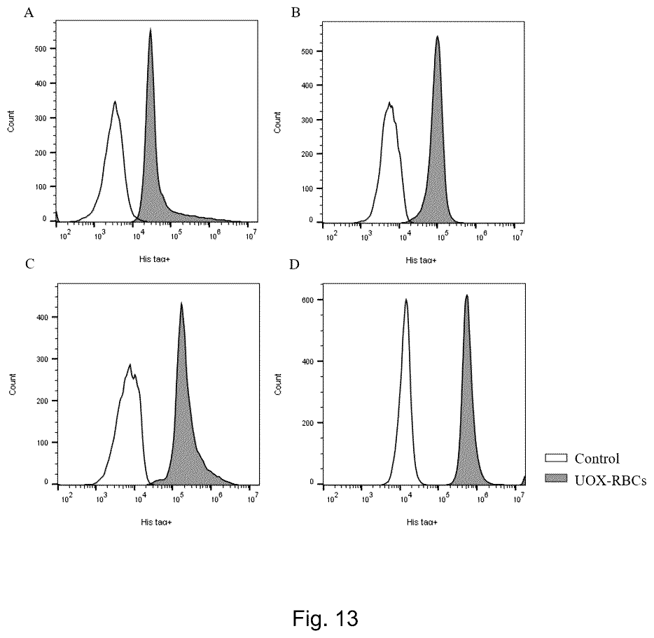

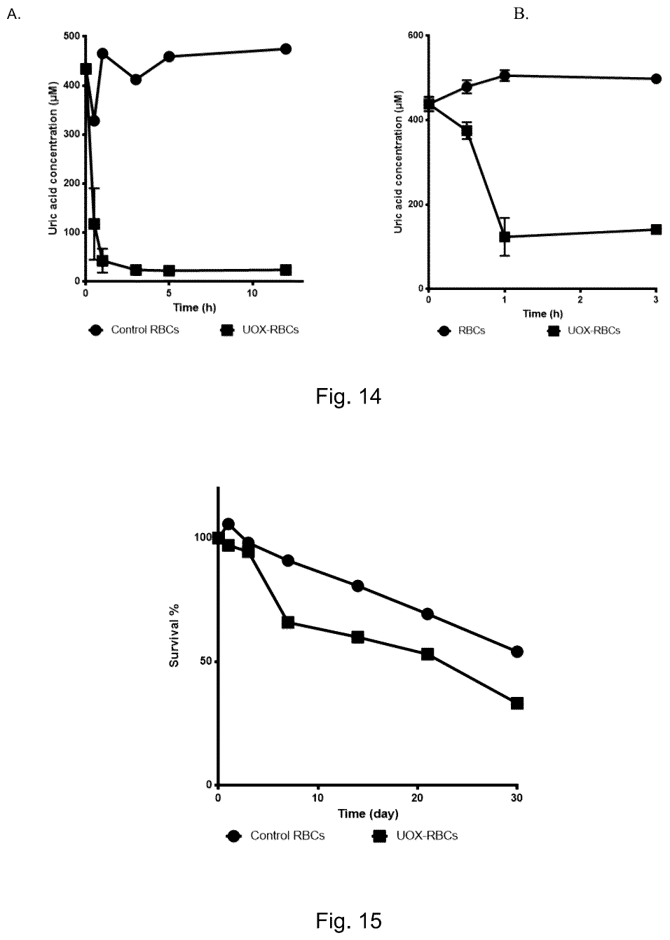

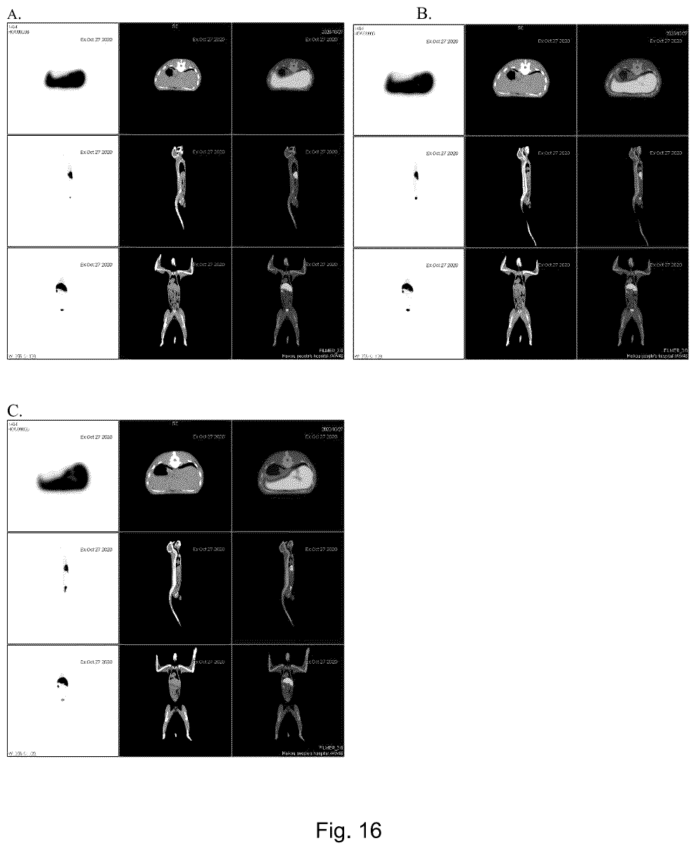

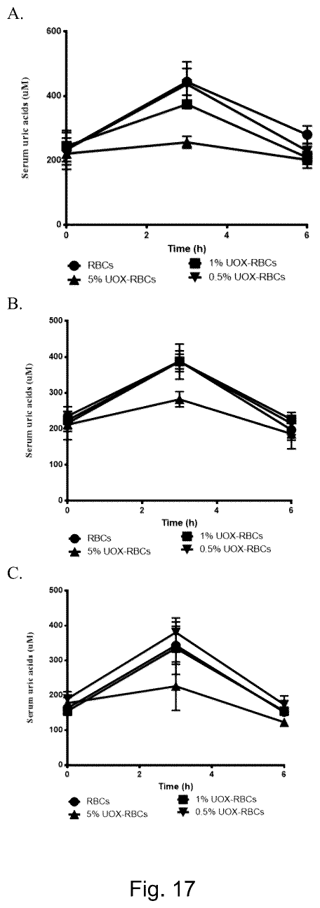

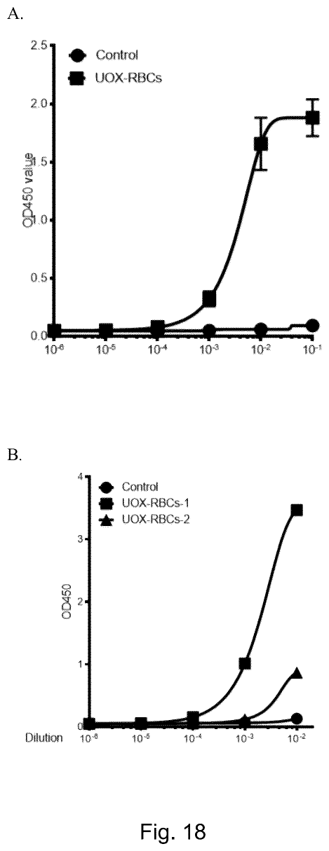

To assess the life-span of these surface modified RBCs in vivo, we next transfused eGFP-cys-6-Mal-LPET*G tagged mouse RBCs, which were simultaneously labeled by a fluorescent dye DiR (1,1′-dioctadecyl-3,3,3′,3′-tetramethylindotricarbocyanine iodide), into wildtype recipient mice. The percentage of DiR and eGFP-cys-6-Mal-LPET*G positive RBCs in vivo was analyzed periodically. We found that eGFP-cys-6-Mal-LPET*G labeled RBCs by mg SrtA not only showed the same lifespan as that of the control groups but also exhibited sustained eGFP-cys-6-Mal-LPET*G signals in circulation for 35 days (FIGS. 9, 10 and 11). Imaging analysis also showed convincing eGFP-cys-6-Mal-LPET*G signals on the cell surface and normal morphology of eGFP-cys-6-Mal-LPET*G tagged RBCs labeled by mg SrtA (FIG. 12).

### Example 3. Mg SrtA-Mediated UOX-Cell Conjugation Via Irreversible Linker

### Methods

Expression and Purification of Mg SrtA in E. coli

Mg SrtA cDNA (SEQ ID NO: 3) were cloned in pET vectors and transformed in E. coli BL21 (DE3) cells for protein expression. Transformed cells were cultured at 37° C. until the OD600 reaching 0.6-0.8 and then 500 μM IPTG were added for 4 hrs at 37° C. After that, cells were harvested by centrifugation and subjected to lysis by precooled lysis buffer (20 mM Tris-HCl, pH 7.8, 500 mM NaCl). The lysates were proceeded for sonication on ice (5s on, 5s off, 60 cycles, 25% power, Branson Sonifier 550 Ultrasonic Cell Disrupter). All supernatants were filtered by 0.22 μM filter after centrifugation at 14,000 g for 40 min at 4° C. Filtered supernatants were loaded onto HisTrap FF 1 mL column (GE Healthcare) connected to the AKTA design chromatography systems. The proteins were eluted with the elution buffer containing 20 mM Tris-HCl, pH 7.8, 500 mM NaCl and 300 mM imidazole. All eluted fractions were analyzed on a 12% SDS-PAGE gel.

Expression and purification of UOX-Cys or UOX-His₆-Cys or UOX-(GS)₃-Cys in E. coli

The coding sequence of UOX (Aspergillus flavus uricase) (SEQ ID NO: 27) was codon optimized for expression in E. coli and synthesized by GenScript. Subclones were generated by standard PCR procedure and inserted into the pET-30a vector with C-terminal His₆ or (GS)₃ linker followed by an additional cysteine residue. All constructs were verified by sequencing and then transformed in E. coli BL21 (DE3) for protein expression.

A single transformed colony was inoculated into 10 ml Luria-Bertani (LB) medium supplemented with ampicillin (100 μg/ml) grown with 220 rpm shaking overnight at 37° C. This 10 ml culture was transferred to 1 L fresh LB medium and the culture was grown with 220 rpm shaking at 37° C. until OD₆(O reached 0.6. The temperature was then lowered to 20° C. and 1 mM IPTG was added for induction.

Cells were harvested at 20 h after induction by centrifugation at 8,000 rpm for 10 min at 4° C. For proteins without the His₆ tag, cell pellet was resuspended in low salt lysis buffer (50 mM Tris 7.5, 50 mM NaCl) and lysed with sonication. The supernatant collected after centrifugation at 10,000 rpm for 1 h was loaded in SP Sepharose FF column (Cytiva, Marlborough, USA) pre-equilibrated with SPA buffer (20 mM Tris 7.5). The column was washed with SPA buffer until the absorbance at 280 nm and conductivity became stable and then eluted using a linear gradient of 0-1 M NaCl in 20 mM Tris 7.5. Fractions corresponding to the elution peak were analyzed by SDS-PAGE and the purest fractions were pooled. To avoid cysteine oxidation, 2 mM TCEP was added to the combined fractions and sample concentration was performed with the use of Amicon Ultra-15 Centrifugal Filter Unit (Millipore, Darmstadt, Germany). Concentrated protein was loaded to EzLoad 16/60 Chromdex 200 μg (Bestchrom, Shanghai, China) pre-equilibrated with PBS, and the target protein peak was collected. For proteins with His₆ tag, cell pellet was resuspended in lysis buffer (50 mM Tris 7.5, 200 mM NaCl, 5 mM imidazole) and lysed with sonication. Tagged proteins were purified over Ni Sepharose 6 FF affinity column (Cytiva) and anion exchange column, followed by size exclusion chromatography. All proteins were stored at −80° C.

The enzymatic activity of UOX-Cys or UOX-His₆-Cys or UOX-(GS)₃—Cys were measured by the decrease in absorbance at 293 nm due to enzymatic oxidation of uric acid using UPLC as described.

The amino acid sequences of UOX-Cys or UOX-His₆-Cys and UOX-(GS)₃—Cys and the nucleic acid sequences encoding UOX-Cys or UOX-His₆-Cys and UOX-(GS)₃—Cys are shown as below:

SEQ ID NO: 29 (Amino acid sequence of UOX-Cys);
MSAVKAARYGKDNVRVYKVHKDEKTGVQTVYEMTVCVLLEGEIETSYTKADNSVIVATDSIK
NTIYITAKQNPVTPPELFGSILGTHFIEKYNHIHAAHVNIVCHRWTRMDIDGKPHPHSFIRD
SEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIET
VEYSLPNKHYFEIDLSWHKGLQNTGKNAEVFAPQSDPNGLIKCTVGRSSLKSKLAAC
SEQ ID NO: 30 (Nucleic acid sequence encoding UOX-Cys):
ATGTCAGCAGTAAAGGCAGCAAGATACGGTAAAGATAATGTCAGAGTCTACAAGGTTCACAAGGACGA
AAAAACTGGTGTTCAAACAGTTTACGAAATGACTGTTTGTGTTTTGTTGGAAGGTGAAATCGAAACTT
CTTACACAAAGGCTGATAACTCAGTTATTGTTGCAACAGATTCTATTAAAAATACTATCTATATCACA
GCTAAGCAAAACCCAGTTACTCCACCAGAATTGTTCGGTTCAATCTTGGGTACACATTTCATCGAAAA
GTACAACCATATCCATGCTGCACATGTTAACATCGTTTGTCATAGATGGACTAGAATGGATATTGATG
GTAAACCACATCCACATTCTTTTATTAGAGATTCAGAAGAAAAGAGAAATGTTCAAGTTGATGTTGTT
GAGGGTAAAGGTATCGATATCAAGTCTTCATTGTCAGGITTAACTGTTTTGAAGTCTACAAATTCACA
ATTTTGGGGTTTCTTGAGAGATGAATACACTACATTGAAGGAAACATGGGATAGAATTTTATCTACTG
ATGTTGATGCTACATGGCAATGGAAGAACTTCTCAGGTTTGCAAGAAGTTAGATCTCATGTTCCAAAA
TTTGATGCTACTTGGGCTACAGCAAGAGAAGTTACTTTGAAGACATTCGCAGAAGATAACTCTGCTTC
AGTTCAAGCAACTATGTACAAGATGGCTGAACAAATCTTGGCAAGACAACAATTGATCGAAACAGTTG
AATATTCATTACCAAATAAGCATTACTTCGAAATCGATTTGTCTTGGCATAAGGGTTTGCAAAACACT
GGTAAAAATGCTGAAGTTTTCGCACCACAATCTGATCCAAATGGTTTGATTAAATGCACAGTCGGTAG
ATCCTCTTTGAAGTCCAAGTTAGCAGCACACCATCATCATCACCATTGCTGA
SEQ ID NO: 31 (Amino acid sequence of UOX-His₆-Cys):
MSAVKAARYGKDNVRVYKVHKDEKTGVQTVYEMTVCVLLEGEIETSYTKADNSVIVATDSIK
NTIYITAKQNPVTPPELFGSILGTHFIEKYNHIHAAHVNIVCHRWTRMDIDGKPHPHSFIRD
SEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVOATMYKMAEQILARQQLIET
VEYSLPNKHYFEIDLSWHKGLQNTGKNAEVFAPQSDPNGLIKCTVGRSSLKSKLAAHHHHHH
C
SEQ ID NO: 32 (Nucleic acid sequence encoding UOX-His₆-Cys):
ATGTCAGCAGTAAAGGCAGCAAGATACGGTAAAGATAATGTCAGAGTCTACAAGGTTCACAA
GGACGAAAAAACTGGTGTTCAAACAGTTTACGAAATGACTGTTTGTGTTTTGTTGGAAGGTG
AAATCGAAACTTCTTACACAAAGGCTGATAACTCAGTTATTGTTGCAACAGATTCTATTAAA
AATACTATCTATATCACAGCTAAGCAAAACCCAGTTACTCCACCAGAATTGTTCGGTTCAAT
CTTGGGTACACATTTCATCGAAAAGTACAACCATATCCATGCTGCACATGTTAACATCGTTT
GTCATAGATGGACTAGAATGGATATTGATGGTAAACCACATCCACATTCTTTTATTAGAGAT
TCAGAAGAAAAGAGAAATGTTCAAGTTGATGTTGTTGAGGGTAAAGGTATCGATATCAAGTC
TTCATTGTCAGGTTTAACTGTTTTGAAGTCTACAAATTCACAATTTTGGGGTTTCTTGAGAG
ATGAATACACTACATTGAAGGAAACATGGGATAGAATTTTATCTACTGATGTTGATGCTACA
TGGCAATGGAAGAACTTCTCAGGTTTGCAAGAAGTTAGATCTCATGTTCCAAAATTTGATGC
TACTTGGGCTACAGCAAGAGAAGTTACTTTGAAGACATTCGCAGAAGATAACTCTGCTTCAG
TTCAAGCAACTATGTACAAGATGGCTGAACAAATCTTGGCAAGACAACAATTGATCGAAACA
GTTGAATATTCATTACCAAATAAGCATTACTTCGAAATCGATTTGTCTTGGCATAAGGGTTT
GCAAAACACTGGTAAAAATGCTGAAGTTTTCGCACCACAATCTGATCCAAATGGTTTGATTA
AATGCACAGTCGGTAGATCCTCTTTGAAGTCCAAGTTAGCAGCATGCTGA
SEQ ID NO: 33 (Amino acid sequence of UOX-GS₃-Cys):
MSAVKAARYGKDNVRVYKVHKDEKTGVQTVYEMTVCVLLEGEIETSYTKADNSVIVATDSIK
NTIYITAKQNPVTPPELFGSILGTHFIEKYNHIHAAHVNIVCHRWTRMDIDGKPHPHSFIRD
SEEKRNVQVDVVEGKGIDIKSSLSGLTVLKSTNSQFWGFLRDEYTTLKETWDRILSTDVDAT
WQWKNFSGLQEVRSHVPKFDATWATAREVTLKTFAEDNSASVQATMYKMAEQILARQQLIET
VEYSLPNKHYFEIDLSWHKGLQNTGKNAEVFAPQSDPNGLIKCTVGRSSLKSKLAAGSGSGS
C
SEQ ID NO: 34 (Nucleic acid sequence encoding UOX-GS₃-Cys):
ATGTCAGCAGTAAAGGCAGCAAGATACGGTAAAGATAATGTCAGAGTCTACAAGGTTCACAA
GGACGAAAAAACTGGTGTTCAAACAGTTTACGAAATGACTGTTTGTGTTTTGTTGGAAGGTG
AAATCGAAACTTCTTACACAAAGGCTGATAACTCAGTTATTGTTGCAACAGATTCTATTAAA
AATACTATCTATATCACAGCTAAGCAAAACCCAGTTACTCCACCAGAATTGTTCGGTTCAAT
CTTGGGTACACATTTCATCGAAAAGTACAACCATATCCATGCTGCACATGTTAACATCGTTT
GTCATAGATGGACTAGAATGGATATTGATGGTAAACCACATCCACATTCTTTTATTAGAGAT
TCAGAAGAAAAGAGAAATGTTCAAGTTGATGTTGTTGAGGGTAAAGGTATCGATATCAAGTC
TTCATTGTCAGGTTTAACTGTTTTGAAGTCTACAAATTCACAATTTTGGGGTTTCTTGAGAG
ATGAATACACTACATTGAAGGAAACATGGGATAGAATTTTATCTACTGATGTTGATGCTACA
TGGCAATGGAAGAACTTCTCAGGTTTGCAAGAAGTTAGATCTCATGTTCCAAAATTTGATGC
TACTTGGGCTACAGCAAGAGAAGTTACTTTGAAGACATTCGCAGAAGATAACTCTGCTTCAG
TTCAAGCAACTATGTACAAGATGGCTGAACAAATCTTGGCAAGACAACAATTGATCGAAACA
GTTGAATATTCATTACCAAATAAGCATTACTTCGAAATCGATTTGTCTTGGCATAAGGGTTT
GCAAAACACTGGTAAAAATGCTGAAGTTTTCGCACCACAATCTGATCCAAATGGTTTGATTA
AATGCACAGTCGGTAGATCCTCTTTGAAGTCCAAGTTAGCAGCAGGTTCTGGTTCTGGTTCT
TGCTGA

Irreversible Linker Conjugation to UOX-Cys or UOX-His₆-Cys or UOX-(GS)₃-Cys by Cysteine Coupling

Irreversible linker, 6-Maleimidohexanoic Acid-Leu-Pro-Glu-Thr-2-hydroxyacetic acid-Gly (6-Maleimidohexanoic Acid-LPET-(2-hydroxyacetic acid)-G, 6-Mal-LPET*G), was synthesized with more than 99% purity. Reactions were performed in a total volume of 1 mL at room temperature for 1 hr in PBS buffer while being rotated at a speed of 10 rpm. The concentrations of 6-Mal-LPET*G and UOX-cys (UOX-His₆ or UOX-(GS)₃—Cys) protein were 2 mM and 500 μM, respectively. This method uses a two-fold molar excess of irreversible linker to UOX-Cys, UOX-His₆-Cys and UOX-(GS)₃—Cys protein. After the reaction, the UOX-Cys-6-mal-LPET*G or UOX-His₆-Cys-6-mal-LPET*G or UOX-(GS)₃-Cys-6-mal-LPET*G products were collected by removal of excess irreversible linker via dialysis and ultrafiltration.

RBC Conjugated with UOX-Cys-6-Mal-LPET*G or UOX-His6-Cys-6-Mal-LPET*G or UOX-(GS)₃-Cys-6-Mal-LPET*G Via Mg Sortase Mediated Reaction

Reactions were performed in a total volume of 200 μL˜15 mL at 37° C. for 2 hrs in PBS buffer while being rotated at a speed of 10 rpm. The concentration of mg SrtA was 10 μM and the UOX-Cys-6-mal-LPET*G or UOX-His₆-Cys-6-mal-LPET*G or UOX-(GS)₃-Cys-6-mal-LPET*G substrates were in the range of 10-100 μM. Human or mouse or rat or cynomolgus monkeys RBCs were washed twice with PBS before the enzymatic reaction. The concentration of RBCs in the reaction was 5×10⁹˜1×10¹⁰/mL.

### The Survival and Stability of the Engineered Red Blood Cells In Vivo

Far red labeled engineered red blood cells were injected into the C57/B6 mice, rats or cynomolgus monkeys through intravenous injection. The survival of the engineered red blood cells in vivo was analyzed by flow cytometry.

### PET-Based Bio-Distribution Analysis

7.5 mL engineered RBCs and 20 mCi fluorodeoxyglucose (18-FDG) were pipetted into a reaction vial and diluted with PBS (67.5 mL). The reaction mixture vial was incubated at 37° C. Following 1 hr incubation, the reaction mixture was purified by centrifugation and the supernatant was removed. Radiochemical yield was determined by radioactivity meter. Finally, the radiolabeled UOX-RBCs were diluted with PBS (15 mL).

At predetermined time points (0.5 h, 1 h and 3 h after the transfusion of engineered RBCs), animals were sedated and imaged by PET-CT (GE Discovery PET/CT Elite) within biosafety level 3 facility. FDG avidity was measured by drawing a region of interest and SUVs (standard uptake volume).

### Analysis of Function of Engineered Red Blood Cells In Vitro

Engineered RBCs were incubated at 37° C. in the presence of 300 μM uric acid. The uric acid concentration was evaluated at specified time to determine the consumption rates of UA by the engineered RBCs in vitro.

### Analysis of Function of Engineered Red Blood Cells In Vivo

We assessed the therapeutic functions of UOX-RBCs in rat model of hyperuricemia. The rats were induced hyperuricemia by hypoxanthine (500 mg/kg) and oxonic acid (250 mg/kg) as described and 1 hr later, functional rat UOX-RBCs (1 mL or 200 μL or 100 μL) were intravenously injected into these rats, analysing their serum UA concentration at 0, 3 and 6 h.

### Immunogenicity Evaluations

Serum samples of rats and cynomolgus monkeys were collected before the transfusion of UOX-RBCs, and 1, 14, 30 days after the transfusion of UOX-RBCs. The amounts of anti UOX/mg SrtA IgG antibodies were measured by enzyme linked immunosorbent assay (ELISA). UOX-Cys-6-mal-LPET*G or UOX-His₆-Cys-6-mal-LPET*G or UOX-(GS)₃-Cys-6-mal-LPET*G or mg SrtA were used as the immobilized antigens to detect the IgG antibodies against the UOX-Cys-6-mal-LPET*G or UOX-His₆-Cys-6-mal-LPET*G or UOX-(GS)₃-Cys-6-mal-LPET*G or mg SrtA, respectively. Serum samples were serially diluted, and end-points were calculated from the highest plasma dilution that showed a positive response (a positive response was defined as an optical density of more than 2.1 times above the mean for the serum samples from the control group at a 490 nm wavelength).

To determine if the immune response was neutralizing, the serum sample were incubated with the UOX for 2 h at 37° C. The enzyme activity was then determined.

### Clinical Observations and Blood and Urine Analysis in the Cynomolgus Monkeys

Clinical signs of mortality were evaluated daily throughout the studies in the cynomolgus monkeys. Body weight were measured weekly and food consumption were measured daily. Blood and urine samples were collected before the transfusion of UOX-RBCs, and 1, 3, 7, 14, 30 days after transfusion of UOX-RBCs. Routine haematology, coagulation, blood biochemistry, and routine urinalysis determinations were carried out using a Siemens Advia 2020i haematology system, a cobas c311 biochemistry analyser, a ThrombolyzerCompactX coagulation analyser and urine analysis test strips.

### Pharmacokinetic Studies

Rats and cynomolgus monkeys were used to examine the pharmacokinetics of UOX-RBCs. The blood samples were collected on the 1st, 3rd, 7th, 14th, 30th day respectively after transfusion of UOX-RBCs. The concentration of UOX in RBCs and plasma was determined by mass spectrometry.

### Results

The purity of UOX-Cys or UOX-His₆-Cys or UOX-(GS)₃—Cys was greater than 90% as judged by SDS-PAGE after purification by Chromdex 200 μg size exclusion column and the resulting UOX-Cys, UOX-His₆-Cys and UOX-(GS)₃—Cys had a specific activity of 10.14, 11.86, 10.58 U/mg as determined (Table 6). The conjugation of irreversible linker (LPET*G) didn't affect the enzyme activity (Table 6).

TABLE 6
Enzyme activity of UOX-Cys or UOX-(GS)₃-Cys or UOX-His₆-Cys or UOX-Cys-6-
mal-LPET*G or UOX-His₆-Cys-6-mal-LPET*G or UOX-(GS)₃- Cys-6-mal-LPET*G

| | | | | UOX-Cys-6- | UOX-(GS)₃-6- | UOX-His₆-Cys- |
|---|---|---|---|---|---|---|
| | UOX-Cys | UOX-(GS)₃-Cys | UOX-His₆-Cys | mal-LPET*G | mal-Cys-LPET*G | 6-mal-LPET*G |
| EAU | 10.14 | 11.86 | 10.58 | 12.1 | 11.6 | 12.4 |

One enzyme activity unit (EAU) corresponds to the enzyme activity that converts 1 μmol of uric acid into allantoin per minute under the operating conditions described: 25° C.±1° C., 50 mM Tris buffer (pH 8.5). The UOX-Cys-LPET*G or UOX-His₆-Cys-LPET*G or UOX-(GS)₃-Cys-LPET*G had a specific activity of 12.1, 11.6, 12.4 U/mg as determined.

We characterized the efficacy of mg SrtA-mediated labeling of UOX on RBC membranes. 5×10⁹-1×10¹⁰/mL mouse (FIG. 13A) or human (FIG. 13B) or rat (FIG. 13C) or cynomolgus monkeys (FIG. 13D) RBCs were incubated with 100 IM UOX-His₆.Cys-LPET*G with or without 10 μM mg SrtA for 2 hrs at 37° C. After the enzymatic reaction, the labeling efficacy was detected by incubating RBCs with PE-conjugated anti-His tag antibody and analyzed by flow cytometry. Our results showed that >99% of natural mouse or human or rat or cynomolgus monkeys RBCs were UOX-labeled by mg SrtA in vitro. In contrast, no significant His tag signal was detected on the surface of mouse or human or rat or cynomolgus monkeys RBCs by the mock control group without mg SrtA enzyme.

We also analyzed the rate of uric acid degradation of UOX-RBCs in vitro. Engineered RBCs were incubated at 37° C. in the presence of ˜400μM uric acid. UOX-RBCs had a uricase activity of 42.12 nmol/h/μL UOX-RBCs, as shown in FIG. 14.

To assess the life-span of these surface modified RBCs in vivo, we next transfused UOX-LPET*G labeled cynomolgus monkeys RBCs, which were simultaneously labeled with a fluorescent dye Far red. The percentage of Far red and His tag positive RBCs in vivo was analyzed periodically. We found that UOX labeled RBCs by mg SrtA showed the same lifespan as the control groups (FIG. 15).

Representative PET images in cynomolgus monkeys following ¹⁸FDG labeled UOX-RBCs injection at various time points are shown in FIG. 16. PET showed most significant distribution in the liver and spleen, while accumulation was low in heart, brain and muscle.

Rat UOX-RBCs reduced UA concentration in a rat model of hyperuricemia following repeated transfusion. The rats were induced hyperuricemia by hypoxanthine (500 mg/kg) and oxonic acid (250 mg/kg) as described previously[Ref 22,23] and 1 hr later, 1 mL (˜5%) or 200 μL (˜1%) or 100 μL (˜0.5%) functional rat UOX-RBCs were intravenously injected into these rats, analysing their serum UA concentration at 0, 3 and 6 h. The UOX-RBCs significantly alleviated the elevated serum UA in the rat model of hyperuricemia (FIG. 17)

In monkeys, positive IgG antibodies reactive to UOX-Cys-6-mal-LPET*G were observed in UOX-RBCs transfused monkeys when the serum samples were diluted to 1:1000. And for rats, positive IgG antibodies reactive to UOX-Cys-6-mal-LPET*G were also observed when the serum samples were diluted to 1:1000, indicating the immunogenicity of UOX-RBCsin both rats (FIG. 18A) and cynomolgus monkeys (FIG. 18B, UOX-RBCs-1 and UOX-RBCs-2 are two replicates). We also determine if the immune response was neutralizing. we incubated these serum samples with UOX for 1 h at 37° C. and there were no differences in enzymatic activity, suggesting that no neutralizing antibodies were present in any of the rats or monkeys.

All cynomolgus monkeys survived to the end of the treatment period. There were no changes considered being related to UOX-RBCs in routine haematology, coagulation, blood biochemistry, and routine urinalysis during the study (see, Tables 7, 8, and 9).

TABLE 7
Haematological parameters in monkeys

| | Quarantine | | | D0 | | | D1 | | | D3 | | |
|---|---|---|---|---|---|---|---|---|---|---|---|---|
| | | UOX-RB | UOX-RB | | UOX-RB | UOX-RB | | UOX-RB | UOX-RB | | UOX-RB | UOX-RB |
| Items | Control | Cs-1 | Cs-2 | Control | Cs-1 | Cs-2 | Control | Cs-1 | Cs-2 | Control | Cs-1 | Cs-2 |
| WBCs | 19.9 | 20.7 | 35.6 | 12.3 | 20.9 | 28.9 | 11.9 | | 15.6 | 8.5 | 9.9 | 12.3 |
| NEUT | 0.9 | 2.4 | 29.6 | 2.5 | 3 | 1.6 | 1.9 | | 0.5 | 1.2 | 1.3 | 0.5 |
| LYM | 4.1 | 3.3 | 3.9 | 2.7 | 3.1 | 5.5 | 3.5 | | 6.7 | 3.3 | 3.4 | 5.7 |
| MONO | 2.4 | 6.9 | 1.7 | 0.3 | 0.7 | 0.5 | 0.3 | | 0.5 | 0.3 | 0.4 | 0.4 |
| EOS | 0.01 | 0.01 | 0.03 | 1.1 | 0.29 | 0.01 | 0.43 | | 0.05 | 0.73 | 0.02 | 0.02 |
| BASO | 0.03 | 0.02 | 0.08 | 0.03 | 0.02 | 0.07 | 0.03 | | 0.04 | 0.02 | 0 | 0.02 |
| RBCs | 6.5 | 6.2 | 6.8 | 6.3 | 5.3 | 6 | 5.7 | | 5.9 | 5.8 | 5.5 | 5.5 |
| HGB | 169 | 138 | 144 | 163 | 116 | 128 | 148 | | 128 | 151 | 120 | 117 |
| HCT | 54.8 | 44 | 47.1 | 53.6 | 37.6 | 43.8 | 47.3 | | 43.6 | 49.9 | 39.2 | 39.6 |
| MCV | 84.4 | 71.2 | 69.4 | 85.2 | 71.2 | 72.6 | 83 | | 73.6 | 86.4 | 71.8 | 71.6 |
| MCH | 26.1 | 22.4 | 21.3 | 25.9 | 22.1 | 21.1 | 26 | | 21.6 | 26.1 | 22.1 | 21.1 |
| MCHC | 309 | 315 | 307 | 304 | 310 | 291 | 313 | | 293 | 302 | 308 | 295 |
| RDW | 14.7 | 14.9 | 14.8 | 15 | 15.6 | 15.3 | 15.3 | | 15.4 | 15.1 | 15.9 | 15.8 |
| HDW | 20.8 | 23.1 | 21.9 | 21.2 | 24 | 21.1 | 22.4 | | 21.6 | 21.6 | 25.9 | 22.5 |
| CHCM | 306 | 312 | 308 | 301 | 313 | 290 | 312 | | 284 | 293 | 307 | 294 |
| CH | 25.7 | 22.1 | 21.3 | 25.7 | 22.2 | 21 | 25.7 | | 20.8 | 25.2 | 21.9 | 20.9 |
| CHDW | | | | | | | | | | | | |
| PLT | 518 | 383 | 336 | 552 | 383 | 343 | 535 | | 346 | 604 | 421 | 343 |
| % RETIC | | | | 1.34 | 1.3 | 0.75 | 1.72 | | 0.8 | 1.93 | 1.54 | 1.17 |
| RETIC | | | | 84.5 | 68.8 | 45.1 | 98.3 | | 47.1 | 111.5 | 84 | 64.9 |
| PCT | | | | | | | | | | | | |
| PDW | | | | | | | | | | | | |
| MPV | 11.1 | 11.8 | 10.7 | 10.1 | 11.8 | 11.2 | 10.4 | | 11.7 | 11.5 | 10.5 | 11 |

| | D7 | | | D14 | | | D21 | | | D30 | | |
|---|---|---|---|---|---|---|---|---|---|---|---|---|
| | | UOX-RB | UOX-RB | | UOX-RB | UOX-RB | | UOX-RB | UOX-RB | | UOX-RB | UOX-RB |
| Items | Control | Cs-1 | Cs-2 | Control | Cs-1 | Cs-2 | Control | Cs-1 | Cs-2 | Control | Cs-1 | Cs-2 |
| WBCs | 13.7 | 21.5 | 19.7 | 12.1 | 20.4 | 17.1 | 9.2 | 17.7 | 10.1 | 11.7 | 10.5 | 18.3 |
| NEUT | 0.5 | 2.2 | 1 | 2.3 | 1.6 | 1 | 1.7 | 2.1 | 0.9 | 1.8 | 2.3 | 1.1 |
| LYM | 4.6 | 3.9 | 5 | 2.9 | 3.4 | 4.6 | 2.8 | 3.4 | 3.5 | 2.5 | 3.1 | 4.6 |
| MONO | 0.8 | 0.9 | 0.7 | 0.4 | 1 | 0.8 | 0.3 | 0.9 | 1.1 | 0.4 | 0.5 | 0.7 |
| EOS | 0.01 | 0.12 | 0.01 | 1.04 | 0.16 | 0.01 | 0.11 | 0.34 | 0.01 | 0.31 | 0.07 | 0.02 |
| BASO | 0.03 | 0.02 | 0.03 | 0.02 | 0.02 | 0.02 | 0.02 | 0.01 | 0.02 | 0.03 | 0.02 | 0.03 |
| RBCs | 6 | 5.2 | 5.5 | 5.6 | 5.2 | 6 | 5.5 | 5.5 | 6 | 5.5 | 5 | 5.9 |
| HGB | 156 | 115 | 118 | 145 | 119 | 133 | 141 | 126 | 132 | 143 | 113 | 129 |
| HCT | 50.4 | 37 | 38.8 | 46.7 | 37.8 | 43.9 | 46.9 | 41.1 | 44.4 | 45.5 | 35.9 | 43.8 |
| MCV | 84 | 71.5 | 71 | 83.4 | 72.8 | 72. | 85.5 | 74.4 | 74 | 83.4 | 72.2 | 74.1 |
| MCH | 26.1 | 22.2 | 21.5 | 25.8 | 22.8 | 22 | 25.7 | 22.7 | 22.1 | 26.2 | 22.8 | 21.8 |
| MCHC | 310 | 310 | 303 | 310 | 314 | 302 | 301 | 305 | 298 | 314 | 316 | 294 |
| RDW | 15.4 | 16 | 16.3 | 15.3 | 16.2 | 16.5 | 14.8 | 15.8 | 15.9 | 15.1 | 15.6 | 15.7 |
| HDW | 22.9 | 25.9 | 25.2 | 23.7 | 25.8 | 26.4 | 23 | 25 | 26 | 23.2 | 24.8 | 24.5 |
| CHCM | 306 | 308 | 297 | 311 | 306 | 294 | 302 | 301 | 287 | 312 | 310 | 286 |
| CH | 25.5 | 21.9 | 21 | 25.8 | 22.2 | 21.2 | 25.6 | 22.2 | 21 | 25.8 | 22.2 | 21.1 |
| CHDW | | | | | | | | | | | | |
| PLT | 704 | 485 | 494 | 544 | 387 | 422 | 475 | 410 | 387 | 493 | 370 | 397 |
| % RETIC | 1.93 | 1.54 | 1.17 | 1.21 | 1.48 | 1.43 | 1.21 | 1.48 | 1.43 | 1.31 | 1.25 | 0.93 |
| RETIC | 111.5 | 84 | 64.9 | 68.3 | 75.8 | 75.2 | 68.3 | 75.8 | 75.2 | 72.7 | 68.4 | 49.3 |
| PCT | | | | | | | | | | | | |
| PDW | | | | | | | | | | | | |
| MPV | 10.6 | 10.5 | 10.9 | 10.1 | 11 | 10.3 | 10.6 | 11.6 | 11.5 | 10.3 | 11.1 | 10.5 |

TABLE 8
Serum biochemistry analysis in monkeys

| | Quarantine | | | D0 | | | D1 | | | D3 | | |
|---|---|---|---|---|---|---|---|---|---|---|---|---|
| | | UOX-RB | UOX-RB | | UOX-RB | UOX-RB | | UOX-RB | UOX-RB | | UOX-RB | UOX-RB |
| Items | Control | Cs-1 | Cs-2 | Control | Cs-1 | Cs-2 | Control | Cs-1 | Cs-2 | Control | Cs-1 | Cs-2 |
| ALI | 92 | 51 | 83 | 68 | 54 | 77 | 63 | | 95 | 58 | 65 | 97 |
| AST | 107 | 74 | 97 | 76 | 52 | 76 | 78 | | 172 | 49 | 51 | 60 |
| ALP | 799 | 430 | 605 | 702 | 388 | 610 | 621 | | 562 | 765 | 411 | 611 |
| GGT | 143 | 139 | 56 | 141 | 112 | 55 | 124 | | 47 | 120 | 100 | 44 |
| CK | 1991 | 937 | 2837 | 1302 | 623 | 1723 | 150 | | 3645 | 272 | 326 | 331 |
| UREA | 5.9 | 7.1 | 6.3 | 6 | 8.3 | 6.4 | 4 | | 5.8 | 4.7 | 6.5 | 5 |
| CREA | 91 | 92 | 104 | 88 | 108 | 85 | 92 | | 97 | 103 | 96 | 92 |
| TP | 85.5 | 84 | 87.4 | 84.6 | 79.9 | 89.9 | 74.4 | | 81.3 | 82.8 | 80.7 | 81 |
| GLU | 4.47 | 3.94 | 3.77 | 3.53 | 8.82 | 4.36 | 5.29 | | 6.71 | 4.4 | 4.08 | 3.73 |
| TBIL | 5 | 1.7 | 2.6 | 4.2 | 3.1 | 1.2 | 3.5 | | 2.5 | 4.9 | 2.3 | 1 |
| TCHO | 3.31 | 3.67 | 3.27 | 3.37 | 3.57 | 3.49 | 2.62 | | 2.99 | 3.95 | 2.98 | 3.14 |
| TG | 0.36 | 0.39 | 0.62 | 0.19 | 0.24 | 0.34 | 0.23 | | 0.49 | 0.23 | 0.48 | 0.33 |
| ALB | 48.6 | 48.9 | 49.9 | 45.8 | 46.9 | 51.3 | 40.5 | | 45.1 | 44.6 | 45.4 | 45 |
| K+ | 7.1 | 4.4 | 4.4 | 5.1 | 4.3 | 4.5 | 6.2 | | 4.9 | 6 | 5.4 | 4.9 |
| Na+ | 152 | 149 | 153 | 151 | 151 | 156 | 148 | | 154 | 154 | 151 | 158 |
| Cl− | 105.3 | 102.9 | 106.4 | 102.2 | 99.2 | 104.1 | 101.7 | | 101.2 | 103.9 | 100.4 | 104.6 |

| | D7 | | | D14 | | | D21 | | | D30 | | |
|---|---|---|---|---|---|---|---|---|---|---|---|---|
| | | UOX-RB | UOX-RB | | UOX-RB | UOX-RB | | UOX-RB | UOX-RB | | UOX-RB | UOX-RB |
| Items | Control | Cs-1 | Cs-2 | Control | Cs-1 | Cs-2 | Control | Cs-1 | Cs-2 | Control | Cs-1 | Cs-2 |
| ALI | 43 | 52 | 61 | 63 | 68 | 70 | 65 | 44 | 47 | 49 | 43 | 63 |
| AST | 57 | 59 | 52 | 54 | 64 | 76 | 43 | 52 | 56 | 50 | 56 | 70 |
| ALP | 779 | 457 | 684 | 692 | 440 | 547 | 683 | 393 | 520 | 709 | 400 | 543 |
| GGT | 117 | 116 | 45 | 118 | 121 | 54 | 125 | 119 | 50 | 109 | 116 | 60 |
| CK | 246 | 301 | 235 | 317 | 394 | 409 | 257 | 404 | 337 | 337 | 367 | 457 |
| UREA | 6.7 | 7 | 5.5 | 6.8 | 7.9 | 6.8 | 5.9 | 7.8 | 6.6 | 5.4 | 7.2 | 5.3 |
| CREA | 99 | 93 | 86 | 85 | 87 | 80 | 90 | 95 | 85 | 103 | 91 | 89 |
| TP | 82 | 79 | 86.6 | 78.6 | 79.6 | 86.8 | 81.1 | 82.1 | 88.7 | 81.4 | 78.8 | 90.6 |
| GLU | 3.43 | 2.4 | 3.11 | 2.71 | 2.95 | 3.24 | 3.78 | 3.47 | 3.36 | 3.04 | 3.52 | 3.23 |
| TBIL | 4.7 | 1.8 | 1.2 | 3.7 | 1.7 | 1.2 | 3 | 1.5 | 2.6 | 2.5 | 1.9 | 1.4 |
| TCHO | 3.31 | 3.09 | 3.12 | 3.38 | 3.95 | 3.34 | 3.63 | 4.16 | 3.63 | 3.47 | 3.65 | 3.53 |
| TG | 0.3 | 0.34 | 0.45 | 0.34 | 0.38 | 0.38 | 0.28 | 0.48 | 0.48 | 0.3 | 0.44 | 0.72 |
| ALB | 43.7 | 44.6 | 47.6 | 43.7 | 44.1 | 48 | 45.2 | 44.6 | 47.8 | 47 | 42.6 | 49.5 |
| K+ | 6.9 | 5.6 | 5.2 | 5.2 | 4.1 | 4.3 | 4.5 | 4.4 | 4.6 | 5.1 | 4 | 3.9 |
| Na+ | 149 | 151 | 154 | 130 | 142 | 147 | 143 | 142 | 144 | 141 | 139 | 142 |
| Cl− | 101.8 | 99 | 1.0 | 98.9 | 98 | 100.7 | 97.1 | 95.1 | 96.5 | 95.4 | 92 | 92.7 |

TABLE 9
Coagulation analysis in monkeys

| | | Quarantine | | | D0 | | | D1 | | | D3 | | |
|---|---|---|---|---|---|---|---|---|---|---|---|---|---|
| | | | UOX-RB | UOX-RB | | UOX-RB | UOX-RB | | UOX-RB | UOX-RB | | UOX-RB | UOX-RB |
| Items | unit | Control | Cs-1 | Cs-2 | Control | Cs-1 | Cs-2 | Control | Cs-1 | Cs-2 | Control | Cs-1 | Cs-2 |
| PT | s | 10.5 | 10.8 | 11 | 10.8 | 12.1 | 11.4 | 10.3 | | 11 | 9.6 | 9.8 | 9.7 |
| INR | — | 0.95 | 0.98 | 1 | 0.98 | 1.11 | 1.04 | 0.93 | | 1 | 0.86 | 0.88 | 0.87 |
| APTT | s | 21.7 | 23.9 | 21.6 | 22.2 | 24.9 | 23.8 | 21.1 | | 22.6 | 18.9 | 22.9 | 20.6 |
| FIB | g/L | 4.57 | 3.71 | 3.87 | 7.39 | 4.26 | 4.12 | 6.63 | | 5.04 | 6.52 | 5.36 | 4.42 |

| | D7 | | | D14 | | | D21 | | | D30 | | |
|---|---|---|---|---|---|---|---|---|---|---|---|---|
| | | UOX-RB | UOX-RB | | UOX-RB | UOX-RB | | UOX-RB | UOX-RB | | UOX-RB | UOX-RB |
| Items | Control | Cs-1 | Cs-2 | Control | Cs-1 | Cs-2 | Control | Cs-1 | Cs-2 | Control | Cs-1 | Cs-2 |
| PT | 10.5 | 11.1 | 10.6 | 11.3 | 11 | 11.8 | 11.4 | 11.7 | 11.5 | 10.9 | 12 | 11.4 |
| INR | 0.95 | 1.01 | 0.96 | 1.03 | 1 | 1.08 | 1.04 | 1.07 | 1.05 | 0.99 | 1.1 | 1.04 |
| APTT | 20.6 | 26.7 | 22.6 | 33 | 26.1 | 21.3 | 21.1 | 24 | 21.4 | 21.1 | 25.7 | 22.7 |
| FIB | 4.72 | 3.87 | 3.72 | 3.32 | 3.25 | 3.2 | 3.42 | 2.97 | 3.39 | 3.92 | 3.71 | 3.87 |

While embodiments of the invention have been illustrated and described, it is not intended that these embodiments illustrate and describe all possible forms of the invention. Rather, the words used in the specification are words of description rather than limitation and it is understood that various changes may be made without departing from the spirit and scope of the invention.

### REFERENCES

[1] J. W. Yoo, D. J. Irvine, D. E. Discher, and S. Mitragotri, “Bio-inspired, bioengineered and biomimetic drug delivery carriers,” Nat. Rev. Drug Discov., vol. 10, no. 7, pp. 521-535, 2011.
[2] J. M. Antos, J. Ingram, T. Fang, N. Pishesha, M. C. Truttmann, and H. L. Ploegh, “Site-Specific Protein Labeling via Sortase-Mediated transpeptidation,” 2017.
[3] J. Shi, L. Kundrat, N. Pishesha, A. Bilate, C. Theile, and T. Maruyama, “Engineered red blood cells as carriers for systemic delivery of a wide array of functional probes,” pp. 1-6, 2014.
[4] P. Daniel Harris, B A, Lynn McNicoll, M D, Gary Epstein-Lubow, M D, and Kali S. Thomas, “Recent Advances in Sortase-Catalyzed Ligation Methodology,” Physiol. Behav., vol. 176, no. 1, pp. 139-148, 2017.
[5] Y. Ge, L. Chen, S. Liu, J. Zhao, H. Zhang, and P. R. Chen, “Enzyme-Mediated Intercellular Proximity Labeling for Detecting Cell-Cell Interactions,” J. Am. Chem. Soc., vol. 141, no. 5, pp. 1833-1837, 2019.
[6] Y. Zhu, T. Guo, and S. K. Sze, “Chapter 22 Elucidating Structural Dynamics of Integral Membrane Proteins on Native Cell Surface by Hydroxyl Radical Footprinting and Nano LC-MS/MS,” vol. 790, pp. 287-303.
[7] Swee, L. K.; Lourido, S.; Bell, G. W.; Ingram, J. R.; Ploegh, H. L. One-step Enzymatic Modification of the Cell Surface Redirects Cellular Cytotoxicity and Parasite Tropism. ACS Chem. Biol. 2015, 10, 460-465.
[8] N. Pishesha et al., “Engineered erythrocytes covalently linked to antigenic peptides can protect against autoimmune disease,” vol. 114, no. 17, 2017.
[9] A. J. Grimm, S. Kontos, G. Diaceri, and X. Quaglia-thermes, “Memory of tolerance and induction of regulatory T cells by erythrocyte-targeted antigens,” Nat. PubL. Gr., pp. 1-11.
[10] A. Karaplis, L. Pong, L. Chien, and N. Chattopadhyay, “Parathyroid hormone ablation alters erythrocyte parameters that are rescued by calcium-sensing receptor gene deletion,” vol. 91, no. 1, pp. 37-45, 2014.
[11]. Garay, R. P., et al., Therapeutic perspectives on uricases for gout. Joint Bone Spine, 2012. 79(3): p. 237-42.
[12]. Wu, X. W., et al., Two independent mutational events in the loss of urate oxidase during hominoid evolution. J Mol Evol, 1992. 34(1): p. 78-84.
[13]. Cortes, J., et al., Control of plasma uric acid in adults at risk for tumor Lysis syndrome: efficacy and safety of rasburicase alone and rasburicase followed by allopurinol compared with allopurinol alone—results of a multicenter phase III study. J Clin Oncol, 2010. 28(27): p. 4207-13.
[14]. Coiffier, B., et al., Efficacy and safety of rasburicase (recombinant urate oxidase) for the prevention and treatment of hyperuricemia during induction chemotherapy of aggressive non-Hodgkin's lymphoma: results of the GRAAL1 (Groupe d'Etude des Lymphomes de l'Adulte Trial on Rasburicase Activity in Adult Lymphoma) study. J Clin Oncol, 2003. 21(23): p. 4402-6.
[15]. Sestigiani E., et al., Efficacy and (pharmaco)kinetics of one single dose of rasburicase in patients with chronic kidney disease. Nephron Clin Pract. 2008; 108(4):c265-71
[16]. Galardy, P. J., et al., Rasburicase in the prevention of laboratory/clinical tumour lysis syndrome in children with advanced mature B-NHL: a Children's Oncology Group Report. Br J Haematol, 2013. 163(3): p. 365-72.
[17]. George, R. J. and J. S. Sundy, Pegloticase for treating refractory chronic gout. Drugs Today (Barc), 2012. 48(7): p. 441-9.
[18]. Sherman, M. R., M. G. Saifer and F. Perez-Ruiz, PEG-uricase in the management of treatment-resistant gout and hyperuricemia. Adv Drug Deliv Rev, 2008. 60(1): p. 59-68.
[19]. Sundy J S, Baraf H S, Yood R A, Edwards N L, Gutierrez-Urena S R, Treadwell E L, Vazquez-Mellado J, White W B, Lipsky P E, Horowitz Z, Huang W, Maroli A N, Waltrip R W 2nd, Hamburger S A, Becker M A. Efficacy and tolerability of pegloticase for the treatment of chronic gout in patients refractory to conventional treatment: two randomized controlled trials. JAMA. 2011 Aug. 17; 306(7):711-20.
[20]. Lipsky P E, Calabrese L H, Kavanaugh A, Sundy J S, Wright D. Wolfson M. Becker M A. Pegloticase immunogenicity: the relationship between efficacy and antibody development in patients treated for refractory chronic gout. Arthritis Res Ther. 2014 Mar. 4; 16(2):R60.
[21]. Ekladious, I., Y. L. Colson and M. W. Grinstaff, Polymer-drug conjugate therapeutics: advances, insights and prospects. Nat Rev Drug Discov, 2019. 18(4): p. 273-294.
[22]. Garcia-Arroyo F E, Gonzaga G, Muñoz-Jiménez I, Blas-Marron M G, Silverio O, Tapia E, Soto V, Ranganathan N, Ranganathan P, Vyas U, Irvin A, Ir D. Robertson C E, Frank D N, Johnson R J, Sánchez-Lozada L G. Probiotic supplements prevented oxonic acid-induced hyperuricemia and renal damage. PLoS One. 2018 Aug. 24; 13(8):e0202901.
[23]. Kurra V, Vehmas T, Eräranta A, Jokihaara J, Pirttiniemi P, Ruskoaho H. Tokola H, Niemelä O, Mustonen J. Pörsti I. Effects of oxonic acid-induced hyperuricemia on mesenteric artery tone and cardiac load in experimental renal insufficiency. BMC Nephrol. 2015 Mar. 27; 16:35.

1. A red blood cell (RBC) having an agent linked thereto, wherein the agent is linked to at least one endogenous, non-engineered membrane protein of the RBC by a sortase-mediated reaction, optionally by a sortase-mediated glycine conjugation and/or a sortase-mediated lysine side chain ε-amino group conjugation, and wherein the agent comprises a uric acid degrading polypeptide.

2. The red blood cell of claim 1, wherein the sortase-mediated glycine conjugation and/or the sortase-mediated lysine side chain ε-amino group conjugation occur at least on glycine₍ₙ₎ and/or lysine ε-amino group at internal sites of the extracellular domain of the at least one endogenous, non-engineered membrane protein, optionally n being 1 or 2.

3. The red blood cell of claim 1, wherein the RBC has not been genetically engineered to express a protein comprising a sortase recognition motif or a nucleophilic acceptor sequence, and optionally, the RBC is a natural RBC, or a natural human RBC.

4. (canceled)

5. The red blood cell of claim 1, wherein the sortase is a Sortase A (SrtA) or as a Staphylococcus aureus transpeptidase A variant (mgSrtA),
optionally wherein the mgSrtA comprises or consists essentially of or consists of an amino acid sequence having at least 60% identity to an amino acid sequence as set forth in SEQ ID NO: 3.

6. (canceled)

7. The red blood cell of claim 1, wherein the agent, before being linked to the RBC, comprises a sortase recognition motif on its C-terminus,
optionally wherein the agent comprises a structure of (A¹-Sp)ₘ-M, in which A1 represents the agent, Sp represents the optional spacer, and M represents the sortase recognition motif; m being an integer greater than or equal to 1, optionally m=1 to 3,
optionally wherein the sortase recognition motif comprises or consists essentially of or consists of an amino acid sequence selecting from a group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS and LPXTA, wherein X is any amino acid,
optionally wherein the sortase recognition motif comprises an unnatural amino acid located at position 5 from the direction of N-terminal to C-terminal of the sortase recognition motif, wherein the unnatural amino acid is an optionally substituted hydroxyl carboxylic acid having a formulae of CH₂OH—(CH₂)ₙ—COOH, n being an integer from 0 to 3, optionally n=0,
optionally wherein M comprises or consists essentially of or consists of an amino acid sequence selecting from a group consisting of LPXT*Y, LPXA*Y, LPXS*Y, LPXL*Y, LPXV*Y, LGXT*Y, LAXT*Y, LSXT*Y, NPXT*Y, MPXT*Y, IPXT*Y, SPXT*Y, VPXT*Y and YPXR*Y, wherein * represents the optionally substituted hydroxyl carboxylic acid; and X and Y independently represent any amino acid;
optionally wherein M comprises or consists essentially of or consists of an amino acid sequence selecting from a group consisting of LPXT*Y, LPXA*Y, LPXS*Y, LPXL*Y, LPXV*Y, LGXT*Y, LAXT*Y, LSXT*Y, NPXT*Y, MPXT*Y, IPXT*Y, SPXT*Y, VPXT*Y and YPXR*Y, wherein * represents the optionally substituted hydroxyl carboxylic acid; and X and Y independently represent any amino acid,
optionally wherein M comprises or consists essentially of or consists of an amino acid sequence selecting from a group consisting of LPXT*G, LPXA*G, LPXS*G, LPXL*G, LPXV*G, LGXT*G, LAXT*G, LSXT*G, NPXT*G, MPXT*G, IPXT*G, SPXT*G, VPXT*G, YPXR*G, LPXT*S and LPXT*A, optionally M is LPET*G with * being 2-hydroxyacetic acid.

8.-12. (canceled)

13. The red blood cell of claim 1, wherein the agent linked to the at least one endogenous, non-engineered membrane protein on the surface of the RBC comprises a structure of (A1-Sp)ₘ-L1-P1, in which L1 is linked to a glycine₍ₙ₎ in P¹, and/or a structure of (A1-Sp)ₘ-L1-P2, in which L1 is linked to the side chain ε-amino group of lysine in P², wherein n is 1 or 2, A1 represents the agent, Sp represents the optional spacer, L′ is selected from the group consisting of LPXT, LPXA, LPXS, LPXL, LPXV, LGXT, LAXT, LSXT, NPXT, MPXT, IPXT, SPXT, VPXT, and YPXR, P¹ and P² independently represent the extracellular domain of the at least one endogenous, non-engineered membrane protein, and X represents any amino acids; m being an integer greater than or equal to 1, optionally m=1 to 3.

14. The red blood cell of claim 5, wherein the Sp is selected from a group consisting of the following types: (1) zero-length type; (2) amine-sulfhydryl type; (3) homobifunctional NHS esters type; (4) homobifunctional imidoesters type; (5) carbonyl-sulfydryl type; (6) sulfhydryl reactive type; and (7) sulfhydryl-hydroxy type;
optionally the one or more Sp is an NHS ester-maleimide heterobifunctional crosslinker, or 6-Maleimidohexanoic acid, 4-Maleimidobutyric acid, and the agent comprises an exposed sulfydryl, optionally an exposed cysteine, optionally a terminal cysteine, optionally a C-terminal cysteine.

15. The red blood cell of claim 1, wherein the uric acid degrading polypeptide comprises one or more polypeptides selected from a group consisting of: uricase, HIU hydrolase, OHCU decarboxylase, allantoinase and allantoicase,
optionally uricase comprising an amino acid sequence set forth in SEQ ID NO: 27 or a functional variant or fragment thereof.

16. The red blood cell of claim 1, wherein the agent additionally comprises a uric acid transporter, which comprises one or more polypeptides selected from a group consisting of: URAT1, GLUT9, OAT4, OAT1, OAT3, Gal-9, ABCG2, SLC34A2, MRP4, OAT2, NPT1, NPT4, and MCT9,
optionally URAT1 comprising an amino acid sequence set forth in SEQ ID NO: 28 or a functional variant or fragment thereof.

17. A composition comprising a plurality of the red blood cells claim 1 and a physiologically acceptable carrier.

18. A method for preparing the red blood cell of claim 1, comprising contacting a red blood cell (RBC) with a sortase substrate that comprises a sortase recognition motif and an agent, in the presence of a sortase under conditions suitable for the sortase to conjugate the sortase substrate to the at least one endogenous, non-engineered membrane protein of the RBC by a sortase-mediated reaction, optionally by a sortase-mediated glycine conjugation and/or a sortase-mediated lysine side chain ε-amino group conjugation, wherein the agent comprises a uric acid degrading polypeptide,
wherein the sortase substrate comprises a structure of (A1-Sp)ₘ-M, in which A¹ represents an agent, Sp represents the optional spacer, and M represents a sortase recognition motif; m being an integer greater than or equal to 1, optionally m=1 to 3,
wherein the sortase recognition motif comprises or consists essentially of or consists of an amino acid sequence selecting from a group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS and LPXTA, wherein X is any amino acid,
optionally wherein the sortase recognition motif comprises an unnatural amino acid located at position 5 from the direction of N-terminal to C-terminal of the sortase recognition motif, wherein the unnatural amino acid is an optionally substituted hydroxyl carboxylic acid having a formulae of CH₂OH—(CH₂)—COOH, n being an integer from 0 to 3, optionally n=0,
optionally wherein M comprises or consists essentially of or consists of an amino acid sequence selecting from a group consisting of LPXT*Y, LPXA*Y, LPXS*Y, LPXL*Y, LPXV*Y, LGXT*Y, LAXT*Y, LSXT*Y, NPXT*Y, MPXT*Y, IPXT*Y, SPXT*Y, VPXT*Y and YPXR*Y, wherein * represents the optionally substituted hydroxyl carboxylic acid; and X and Y independently represent any amino acid,
optionally wherein M comprises or consists essentially of or consists of an amino acid sequence selecting from a group consisting of LPXT*G, LPXA*G, LPXS*G, LPXL*G, LPXV*G, LGXT*G, LAXT*G, LSXT*G, NPXT*G, MPXT*G, IPXT*G, SPXT*G, VPXT*G, YPXR*G, LPXT*S and LPXT*A, optionally M is LPET*G with * being 2-hydroxyacetic acid.

19.-23. (canceled)

24. The method of claim 11, wherein the Sp is selected from a group consisting of the following types: (1) zero-length type; (2) amine-sulfhydryl type; (3) homobifunctional NHS esters type; (4) homobifunctional imidoesters type; (5) carbonyl-sulfydryl type; (6) sulfhydryl reactive type; and (7) sulfhydryl-hydroxy type;
optionally the one or more Sp is an NHS ester-maleimide heterobifunctional crosslinker or 6-Maleimidohexanoic acid or 4-Maleimidobutyric acid, and the agent comprises an exposed sulfydryl, optionally an exposed cysteine, optionally a terminal cysteine, optionally a C-terminal cysteine.

25. A method for treating or preventing a disorder, condition or disease associated with an elevated uric acid level in a subject in need thereof, comprising administering the composition of claim 10 to the subject.

26. The method of claim 13, wherein the subject has a serum uric acid level greater than about 8.0 mg/dl prior to the administering.

27. The method of claim 13, wherein the disorder, condition or disease associated with an elevated uric acid level is selected from a group consisting of hyperuricemia, gout (chronic refractory gout, gout tophus and gouty arthritis), metabolic syndrome, tumor lysis syndrome, Lesch-Nyhan syndrome, cardiovascular disease, diabetes, hypertension, renal disease, and uric acid nephrolithiasis.

28.-31. (canceled)
